SEQUENCE LISTING (44 sequences)

SEQ ID NO: 1 - protein, 1045 aa, synthetic construct
MKKVEVSRPYQSLLLPNHRKFKYLDETWNAYKSVKSLLHRFLVCAYGAVPFNKFVEVVEKVDNDQLVLAFAVRLFRLVPVESTSFAKVDKANLAKSLANHLPVGTAIPANVQSYFDSNFDPKKYMWIDCAWEADRLAREMGLSASQFSEYATTMLWEDWLPLNKDDVNGWGSVSGLFGEGKKEDRQQKVKMLNNLLNGIKKNPPKDYTQYLKILLNAFDAKSHKEAVKNYKGDSTGRTASYLSEKSGEITELMLEQLMSNIQRDIGDKQKEISLPKKDVVKKYLESESGVPYDQNLWSQAYRNAASSIKKTDTRNFNSTLEKFKNEVELRGLLSEGDDVEILRSKFFSSEFHKTPDKFVIKPEHIGFNNKYNVVAELYKLKAEATDFESAFATVKDEFEEKGIKHPIKNILEYIWNNEVPVEKWGRVARFNQSEEKLLRIKANPTVECNQGMTFGNSAMVGEVLRSNYVSKKGALVSGEHGGRLIGQNNMIWLEMRLLNKGKWETHHVPTHNMKFFEEVHAYNPSLADSVNVRNRLYRSEDYTQLPSSITDGLKGNPKAKLLKRQHCALNNMTANVLNPKLSFTINKKNDDYTVIIVHSVEVSKPRREVLVGDYLVGMDQNQTASNTYAVMQVVKPKSTDAIPFRNMWVRFVESGSIESRTLNSRGEYVDQLNHDGVDLFEIGDTEWVDSARKFFNKLGVKHKDGTLVDLSTAPRKAYAFNNFYFKTMLNHLRSNEVDLTLLRNEILRVANGRFSPMRLGSLSWTTLKALGSFKSLVLSYFDRLGAKEMVDKEAKDKSLFDLLVAINNKRSNKREERTSRIASSLMTVAQKYKVDNAVVHVVVEGNLSSTDRSASKAHNRNTMDWCSRAVVKKLEDMCNLYGFNIKGVPAFYTSHQDPLVHRADYDDPKPALRCRYSSYSRADFSKWGQNALAAVVRWASNKKSNTCYKVGAVEFLKQHGLFADKKLTVEQFLSKVKDEEILIPRRGGRVFLTTHRLLAESTFVYLNGVKYHSCNADEVAAVNICLNDWVIPCKKKMKEESSASG

SEQ ID NO: 2 - protein, 1079 aa, synthetic construct
MPKQKDLPYSCLSYLQPNERKLKLLNNTYDHLTHGSKIMFDTLIALMGGINPKMDVISENKDSEIKNNRDPQTMCATIWFRPMKSKRINKVWSPKQLKEQFLKYYQEYEADVKINDMVEAYFDSPLGENYVWVDCRKKYKQLVKELASIAKTTEANLKEDLDCDLECLFRPSEKKMKLYGSNKSWAIISNLFGEGDKEDRSKKIKILTKAIQILTESNPESYADVQKAFLAAADIDDPKKFHTQEIWGNGSPGNIVKMARGDFLGKEFDCEKILEKINDVLKEKTLDFDLKVRLSFKEYLISKIGHYYQNSWSEMINSAFADIISKNTRNVNFAKEKVQLQKTLSETSNAKVELLTDFFKSDFFLGDDKFDIAPHNLGGANGIKFFYDFCKKNEDQYFLEELLLEAAIEESVAEAKSKSLKEPHKDLLRYVFSIRKETTFEELRDAAKYIQTHKRIKNMSVHPTVKSDIGFNVTSGSALVGHVVSPSKKINGRIAGESGFIWICMKLWEGGDKWIEHHIPFTDTRFYEQIYKYNPDSKLEPVVLRTKRYGVDLTKFNLPPMKTDLKHVAPKEKNKHNYVKVQRRLQRLNHPDVPNTIWPKSNIGFTIRRKNGKYILNVVHKLPKNKVKKSVKPKFGDILIGVDQNQTTNHTCSIYKVVKKNTKEALLVPESDFYLKKIETIKVTSFTKARYNSEPIDQLHYEGISVDNEVFKNWCKDREQFVDSLSIKEFKNEFKRIKNKNENLYSFNADYLWLLKRIISGKLNKKKFDVSVFEKSIRNEILAMCSKEGLGPLRVSSLSSNSLKSIGFLKSAICSFISIALNRKGIEDKTDVQKNKIDPELFDLIGKIEQKRVNKRMEKTRRNADFILTMAVDYQKSSQKNVFLFCEGNLETAKTGNSKKRNSANVDWCSRKLFDFLKEKSLRHGIYFHAVTPHYTSHQDPFEYHPSNKVMLPRFAKFDKNNPIQDWAEKKYLGFANSDPESGTALYYKKGVENFFAHYQKGFKEKVELAEMKNVLNSNLKNGNLEHVFCPIRGGRYYLSTHPVTSDAKPFEFNGRKCYICDSDEVAATNIMLIGLFYV

SEQ ID NO: 3 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 4 - DNA, 3135 bp, synthetic construct
  source 1..3135: mol_type = other DNA
atgaagaaggttgaggtgtcccgcccgtaccagtccctcctgctgccgaaccataggaagttcaagtacctggacgaaacctggaacgcttacaagtccgtgaagagcctcctgcaccgcttcctggtgtgtgcctacggcgccgtgccgttcaacaagttcgtggaggtggttgagaaggtggacaacgaccagctggtgctcgccttcgccgtgcgcctgttcaggctggtgccggttgagagcacctcattcgccaaggtggacaaggccaacctggccaagagcctcgccaaccacctcccggtgggcaccgccatcccggccaacgtgcagtcctacttcgactccaacttcgacccgaagaagtacatgtggatcgactgcgcttgggaggccgacaggctggccagggagatgggcctctcagcctcacagttcagcgagtacgccaccaccatgctgtgggaggactggctcccgctcaacaaggacgacgtgaacggctggggctccgtgtccggcctgttcggcgagggcaagaaggaggacaggcagcagaaggtgaagatgctcaacaacctcctgaacggcatcaagaagaacccgccgaaggactacacccagtacctgaagatcctgctcaacgccttcgacgccaagagccacaaggaggccgtgaagaactacaagggcgactccaccggccgcaccgcctcctacctgagcgagaagtccggcgagatcaccgaactcatgctggagcagctgatgtccaacatccagcgcgacatcggcgacaagcagaaggagatcagcctcccgaagaaggacgttgtgaaaaaatacctggagtccgagagcggcgtgccgtacgaccagaacctctggtcccaggcctacaggaacgccgcctcctcaatcaagaaaactgacaccaggaacttcaacagcaccctggagaagttcaagaacgaggtggagctgcgcggcctgctgtcagagggcgacgacgtggagatcctcaggagcaagttcttctcctccgagttccacaagactccggacaagttcgtgatcaagccggagcacatcggcttcaacaacaagtacaacgtggtggctgagctgtacaagctcaaggccgaggccactgacttcgagtcagccttcgccaccgtgaaggacgagttcgaggagaagggcatcaagcaccctatcaagaacatcctggagtacatctggaacaacgaggtgcctgtggagaagtggggccgcgtggctaggttcaaccagtcagaggagaagctgctcaggatcaaggccaacccgaccgtggagtgcaaccaaggcatgaccttcggcaactccgccatggttggcgaggtgctgaggtccaactacgtgagcaagaagggcgctctcgtgagcggcgagcacggcggccgcctcatcggccagaacaacatgatctggctcgaaatgcgcctcctgaacaagggcaagtgggaaacccaccacgtgccgacccacaacatgaagttcttcgaggaggtgcacgcctacaacccttcactcgccgacagcgtgaacgtgaggaacaggctctacaggtccgaggactacacccagctcccttcctccatcactgatggcctgaagggcaacccgaaggccaagctcctgaagcgccagcactgcgccctcaacaacatgaccgccaacgtgctgaacccgaagctgagcttcaccatcaacaagaagaacgacgactacaccgtgatcatcgtgcactcagtggaggtgtccaagccgaggcgcgaggtgctcgtgggcgactacctggtgggcatggaccagaaccagaccgccagcaacacctacgccgtgatgcaggtcgtgaagcctaagagcaccgacgccatcccgttcaggaacatgtgggtgaggttcgtggagtccggctccatcgagtcccgcaccctcaacagcaggggcgagtacgtggaccagctgaaccacgacggcgttgacctcttcgagatcggcgataccgagtgggtggattccgcccgcaagttcttcaacaagctcggcgttaagcacaaggatggcaccctcgtggacctctccaccgccccgcgcaaggcctacgccttcaacaacttctacttcaagactatgctcaaccatctccgcagcaacgaggtggacctcaccctgctgaggaacgagatcctgcgcgttgccaacggcaggttctccccgatgaggctgggctcactcagctggactaccctgaaggccctcggcagcttcaagagcctcgtgctcagctacttcgaccgcctgggcgccaaggagatggtggacaaggaggccaaggataagtccctcttcgacctgctggtggccatcaacaacaagaggtccaacaagagggaggagcgcaccagccgcatcgccagcagcctgatgaccgtggctcagaagtacaaggtggacaacgccgttgttcatgtggtggtggagggcaacctcagctccaccgaccgctcagccagcaaggcccacaaccgcaacaccatggactggtgcagccgcgccgtggtgaagaagctggaggacatgtgtaacctgtacggcttcaacatcaagggcgtgccggccttctacacctcccatcaggatccgctggtgcacagggctgactacgatgacccgaagccggctctccgctgcaggtactccagctactcacgcgccgacttctccaagtggggccagaacgccctcgctgccgtggtgcgctgggctagcaacaagaagagcaacacgtgctacaaggttggcgccgtggagttcctgaagcagcacggcctgttcgccgataagaagctcactgttgagcagttcctgtccaaggtgaaggacgaggagatcctcatcccgaggcgcggcggcagggtgttcctgaccacccaccgcctcctcgccgagagcaccttcgtgtacctcaacggcgttaagtaccacagctgcaacgccgacgaggtggctgccgtgaacatctgcctgaacgattgggtgatcccgtgcaagaagaagatgaaggaggagtccagcgccagcggc

SEQ ID NO: 5 - DNA, 3135 bp, synthetic construct
  source 1..3135: mol_type = other DNA
atgaagaaggtggaggtgtccaggccctaccagtccctgttgctccccaaccacagaaagttcaagtatctggacgaaacctggaacgcctacaagtcagtcaagtccctgctccacaggttccttgtgtgcgcctacggcgctgtgcccttcaacaaattcgttgaggtcgtcgagaaggtcgacaacgatcagctcgtcctcgcctttgccgtgagactcttcaggctggtgcccgtggaatccacttccttcgccaaggtcgacaaagctaacttggctaaatcactggccaatcacctccctgtgggcaccgccatccctgccaatgtgcaatcctacttcgactccaacttcgaccccaagaagtacatgtggatagactgtgcctgggaagccgataggttggccagggagatgggcctctccgcctcccaattttccgagtacgccaccaccatgctgtgggaggactggcttccccttaacaaagatgacgtcaacggatggggctctgtttctggtctgttcggcgaaggcaagaaggaggacaggcagcagaaagtcaagatgctgaataacctgctcaacgggataaagaagaacccccccaaggactacacacagtacctcaagatcctcctgaacgccttcgatgccaagtcccacaaagaggccgtgaagaactataaaggggacagtaccggcaggaccgcctcctatttgtccgagaagtccggggagatcacagagctgatgctggaacagttgatgagtaatatccagagggacattggagacaagcagaaagaaatatccctccccaagaaggacgttgtgaaaaaataccttgagtccgagtccggtgtcccctacgaccagaacttgtggtcccaggcctacaggaacgccgcttcttccattaagaaaaccgataccaggaactttaactctaccctggagaagttcaagaatgaggttgagctgaggggtctgttgtccgagggcgacgacgttgagatactgagatccaagttcttttcctccgaattccacaagactcccgacaaattcgtgatcaagcccgagcacatcgggtttaacaacaagtataacgttgtcgccgaactttacaagcttaaggccgaggccaccgatttcgagtcagcctttgccaccgtgaaggacgagtttgaggagaagggcatcaaacaccccatcaaaaacattctggaatacatctggaacaacgaggttcccgttgaaaagtggggcagggtcgctaggttcaaccagtcagaagagaagctgctcaggatcaaggcaaaccccaccgtcgagtgcaaccaagggatgaccttcgggaactccgccatggttggcgaggtgctcaggtccaactacgtgtccaaaaagggcgccctggtgtccggagagcacggcgggaggttgatcgggcagaataacatgatttggctggagatgaggctcctgaacaaagggaagtgggagactcaccatgttcccacacacaacatgaaattcttcgaagaggttcatgcctataacccctctctcgccgactccgtcaatgtgaggaacaggctgtacaggtccgaggactacacccagctgcctagttccatcaccgacggccttaaagggaatccaaaggccaagctcttgaagaggcagcattgtgccctgaataacatgaccgcaaacgtcctgaacccaaagctcagtttcaccatcaataagaagaatgatgactatactgttatcatcgttcactccgttgaggtgtccaagccaaggagagaggtcttggtgggagactacctcgtcgggatggaccagaaccagaccgcatccaatacctacgccgtcatgcaggtcgttaagcccaaatccaccgacgccatccccttcaggaatatgtgggtcaggttcgtcgagtccggctcaatcgagtccaggacactgaactctaggggggagtacgttgatcaactcaatcacgatggtgtggacctcttcgagatcggcgacacagagtgggtcgactccgcaaggaagttcttcaacaaactcggggtgaaacacaaggacggaaccctggtggacctcagtactgcacccagaaaagcctacgcttttaacaatttctacttcaagaccatgctgaaccatctgagatccaacgaagtggacttgaccttgctcaggaacgagatcctcagagtggccaacgggaggttttcacctatgaggctgggcagtctctcctggaccacccttaaggctctggggtcctttaagtccctggttctttcctatttcgacaggctcggggccaaagagatggtggacaaggaggccaaggataagtccctcttcgacctcctcgtggccatcaataacaaaaggtccaacaagagagaggagaggacctccagaatcgcctcctctcttatgaccgtggctcagaaatacaaggtcgacaacgcagtggttcacgtcgtcgttgaaggtaacctgtcctccaccgacaggtcagcctccaaggcccacaatagaaacaccatggactggtgctccagggccgttgtcaagaagctggaggatatgtgcaacctctacgggttcaacatcaagggggtcccagctttctacacctcccaccaagatcccctggtccacagggccgactacgacgatcccaagcccgccttgagatgcaggtactcctcctactccagagccgattttagtaagtggggtcagaacgccctggccgccgtggtgagatgggcctccaataaaaagtccaacacgtgctataaagtcggcgccgtggagttcctcaagcaacatgggctcttcgcagacaagaagctgacagtcgaacaatttctctccaaggtgaaggatgaagagatcctcatccccaggagaggcggaagggtgttccttaccacccacaggctccttgcagaatccactttcgtttatctgaacggtgtcaagtaccattcctgtaacgcagacgaggttgccgccgtgaatatctgcctgaacgactgggtcatcccctgcaaaaagaagatgaaggaagagtcctccgcctccggc

SEQ ID NO: 6 - RNA, 23 nt, synthetic construct
  source 1..23: mol_type = other RNA
agagaatgtgtgcatagtcacac

SEQ ID NO: 7 - RNA, 24 nt, synthetic construct
  source 1..24: mol_type = other RNA
agaaaatgtgtcatactacgacac

SEQ ID NO: 8 - RNA, 23 nt, synthetic construct
  source 1..23: mol_type = other RNA
agaaatccgtctttcattgacgg

SEQ ID NO: 9 - DNA, 3237 bp, synthetic construct
  source 1..3237: mol_type = other DNA
atgcccaaacagaaggatctcccctactcctgcctgtcctacctgcagcctaatgagaggaagttgaaacttctgaataacacctacgaccacctcacccatggctccaaaatcatgtttgacacactcatcgccctcatgggcgggataaatccaaagatggatgtcatctccgagaacaaagattccgagataaagaacaacagggatccccagacaatgtgcgccaccatctggttcaggcccatgaagtccaaaagaataaacaaggtttggtcccccaagcagctcaaggagcagttcctcaagtactaccaggagtacgaagccgacgtcaaaatcaatgacatggtcgaggcctacttcgattcccccctcggcgagaactacgtttgggtcgactgtaggaagaagtataagcagctcgtcaaggagctggcatccatcgccaagaccaccgaggccaatctcaaggaggacctggactgtgacctggagtgcctgttcaggccttccgaaaagaagatgaaattgtacggcagtaacaaaagttgggccatcatctccaacctcttcggcgaaggtgacaaggaggacaggtccaagaagatcaagatcctgaccaaggcaattcagatcctcacagagtctaaccccgagtcctacgctgacgtccagaaggcctttcttgccgccgccgatatcgatgaccccaaaaagttccacacccaagagatctggggtaacgggtcccccggaaacatcgttaaaatggccagaggggactttctgggcaaggaattcgattgcgagaagatcctggagaaaattaacgacgtcttgaaggaaaagaccctggatttcgacctgaaggtgagactctccttcaaggaatatctgatctccaagattgggcactactatcaaaattcctggagtgagatgattaactccgccttcgccgacatcatctcaaagaacaccaggaacgtcaacttcgccaaagagaaagtccagctccagaagactctttctgagacatccaacgccaaggttgagcttcttaccgacttcttcaagtccgacttcttccttggtgacgataaattcgacatcgcaccccacaaccttggcggcgccaacggcattaagttcttctacgatttctgtaagaagaacgaggaccagtacttcctggaggagttgctcctcgaagccgccatcgaggagtccgttgccgaggccaagtccaagtccctcaaggagccccacaaagacctgctcaggtacgtcttctcaatcaggaaggagaccaccttcgaggagctgagagacgccgccaagtatatccaaacccataagaggatcaaaaacatgtccgtccaccccaccgttaaatccgacatcggctttaacgtgacatccggctccgccctcgtggggcacgtcgtgtccccatccaaaaaaattaacgggaggatagccggcgagtccggctttatctggatttgcatgaagctctgggagggcggagacaagtggatcgaacaccatatccccttcaccgacaccaggttctatgagcagatctataaatataaccccgactccaagctcgaacctgtggtgctcaggaccaagagatatggcgtcgaccttaccaagttcaatctgccccccatgaagaccgacctgaagcatgttgcccccaaagagaaaaacaagcacaattacgtgaaggttcagaggaggctccagaggctcaaccatccagacgttcccaacaccatttggccaaagtctaacattgggttcactatcaggagaaagaacgggaaatacatcctcaacgtcgtgcataaactccctaagaataaggtgaagaagagtgtgaagcccaagtttggggatattttgatcggtgtggaccagaatcaaaccaccaaccacacctgctccatctacaaggttgttaagaagaacactaaagaggccctgctcgtgcctgagtctgacttctaccttaagaagatcgaaaccatcaaagtcacttccttcaccaaagccaggtacaactccgagcccatagaccagctgcactacgagggcatctccgtggacaacgaggtcttcaagaattggtgcaaggacagagaacaatttgtggacagtctttccatcaaggagttcaagaacgagttcaagagaattaaaaataagaatgaaaacctctactccttcaacgccgactacttgtggctcctcaagaggatcatctccggcaagttgaacaagaagaagttcgatgtgtccgttttcgagaaatccataaggaatgagatcctggccatgtgctcaaaggaagggcttgggcccctgagggtctcctccctgtcctccaactcccttaaaagcattgggtttctcaagtccgcaatctgctccttcatctcaatcgctctgaacaggaaggggatcgaggacaagaccgacgttcagaagaacaagatcgatcccgagctgtttgacctcatcggtaagatagagcaaaagagggttaacaagaggatggagaagaccaggaggaacgccgatttcatactgactatggccgtcgactaccagaagtcctcacaaaagaatgtgtttctgttctgcgaagggaacctggagactgcaaaaaccgggaacagtaaaaagaggaactccgccaacgtcgactggtgctctaggaaattgttcgactttctgaaagaaaaatccctgagacacgggatctacttccacgctgtgacaccccactacacctcccaccaggatccctttgagtaccacccctccaacaaggttatgctcccaaggttcgccaagttcgacaagaacaaccccatacaggactgggccgagaaaaaatatttgggattcgctaactccgaccccgagtccggtaccgctctgtactataaaaaaggagtggaaaacttttttgcccactaccagaagggtttcaaggaaaaagtcgaactggctgagatgaaaaacgtcctcaattccaacctcaaaaatggaaatctggagcacgtgttttgccccatcaggggggggaggtattatttgtcaacccatcccgtgacctctgacgccaagcccttcgagttcaacggaagaaagtgttacatctgcgactccgacgaagtggccgcaaccaacatcatgctgatcggcttgttctatgtg

SEQ ID NO: 10 - DNA, 3162 bp, synthetic construct
  source 1..3162: mol_type = other DNA
atgtcatccgccatcaaatcctacaagtccgtgctcagacccaacgagaggaagaaccagctcctgaagagtaccatccaatgcctcgaggacgggtccgccttttttttcaaaatgttgcagggactttttggcggcattacccccgagatcgttaggttctccacagaacaggaaaaacaacaacaggacatcgccttgtggtgcgccgtgaactggttcaggcctgtttcccaagactccctgacccacactatcgcatccgacaacctcgtcgaaaagttcgaagagtactacggggggaccgcatccgacgccatcaagcagtatttcagtgcctccattggcgaaagttactattggaacgactgtaggcagcaatactacgatttgtgcagggagctgggcgtcgaggtgtccgaccttacacacgacctcgaaattctctgcagggagaagtgcttggctgtggccaccgagtccaaccagaacaattccattatcagtgtcctgttcgggaccggggaaaaggaggacagatctgttaaactgaggatcaccaaaaagatcttggaggccatctctaacctgaaggaaatacccaagaacgtcgccccaattcaggagatcatcctgaatgtggccaaagccaccaaagagaccttcaggcaggtgtatgccggcaacctgggcgccccctccacactcgagaaattcatcgccaaagacggtcagaaagagttcgaccttaagaagctgcagaccgacctcaaaaaggtgatcaggggaaagtccaaagagagggattggtgctgtcaggaagagctgaggtcctacgtcgagcaaaatacaatccaatacgatctttgggcatggggggagatgtttaacaaggcccacactgccctcaaaattaaatccaccaggaactacaacttcgccaagcagagactggagcaattcaaggagatccagtccttgaataatctcctcgtggttaagaagctcaatgatttcttcgattccgagttcttctccggagaggagacctacaccatctgcgttcatcacctcggcggaaaggacctgtccaagctgtacaaggcctgggaagacgaccccgcagaccccgagaacgccatcgtcgtgctctgcgacgacctcaagaacaactttaagaaagagccaatcaggaacatccttagatacatctttaccataaggcaagagtgctccgcccaggacatcctggccgccgctaaatataaccagcagctcgacaggtataaaagccagaaggcaaacccatcagtgctggggaaccaagggttcacctggaccaacgccgttatcctgcccgaaaaagcccagaggaacgataggcccaattccctggacctcagaatctggctttatttgaagctgaggcaccccgacggtagatggaagaaacaccatatccccttttacgacactaggtttttccaagagatctacgccgccggaaactcccccgtggacacctgccaattcaggacccctaggttcggttaccacctccccaagctgacagaccagaccgccatcagggtgaacaagaagcatgttaaggccgctaagaccgaagccaggataaggctcgccatccagcagggtaccctgcccgtctcaaatttgaagatcacagagatatccgccactatcaactctaagggccaggtcaggatcccagtgaagttcgacgttggcaggcagaagggtaccctccaaatcggcgacaggttctgcgggtacgaccagaaccagactgcatcccacgcttacagtttgtgggaggtcgtgaaggagggacagtatcacaaggagctgggctgcttcgtgagattcatctcatccggggatattgtctccatcaccgagaacaggggcaatcagttcgaccagttgtcatatgaggggctggcttaccctcagtacgccgactggaggaagaaagcatccaaattcgtctccctttggcagatcacaaagaagaacaagaagaaggagatcgtcactgtcgaggccaaggagaagttcgacgctatctgcaagtatcagcccaggctgtacaagttcaacaaagagtacgcctatctcctgagggacatcgtcagagggaagtccctcgttgagctccagcaaataaggcaggagatcttcagatttatcgagcaagactgcggggtgaccaggctggggtctctgtccctgtccactcttgaaaccgtcaaggccgtgaagggcatcatctactcctacttctccaccgccctcaacgcctcaaaaaataaccccatctccgatgagcagaggaaggagttcgatcccgagcttttcgccctcctcgagaagctggagctcatcagaaccagaaagaaaaaacagaaggttgagaggatcgcaaactccctcatacagacctgtttggagaacaacattaagtttatcaggggcgagggcgatctctccaccaccaacaacgccaccaagaagaaggccaattccaggtccatggattggttggctaggggcgtcttcaacaagatcagacagctggcccccatgcacaacattaccctctttggttgcggctccctttatacctcccaccaggaccccctcgtccacaggaaccccgataaggctatgaaatgcaggtgggccgctatccccgtgaaggacatcggggactgggttcttaggaagctgtcccagaatctcagggccaagaatatcggcaccggagagtactaccaccaaggggtgaaggaattcctctcccattacgagctgcaggacctcgaagaggagctccttaagtggaggtccgatagaaagtccaacataccctgttgggtcctgcagaacaggctcgccgaaaaactggggaacaaggaggccgttgtctacatccctgttagaggtgggaggatctacttcgccacccataaagtcgccaccggggccgtttccatagtttttgatcagaaacaggtgtgggtctgtaacgccgaccatgtggccgccgcaaatattgccctgaccgtcaaggggataggcgaacagtcttccgacgaagagaaccccgacggctccagaattaaactgcagctgacctcc

SEQ ID NO: 11 - DNA, 3237 bp, synthetic construct
  source 1..3237: mol_type = other DNA
atgccgaagcagaaggatctgccgtacagctgtctgtcctacctccagccgaacgagaggaagctgaagctgctcaacaacacctacgatcacctgacccacggcagcaagatcatgttcgacaccctcatcgccctcatgggcggcatcaacccgaagatggacgtgatctccgagaacaaggacagcgagatcaagaacaacagggacccgcagaccatgtgcgccaccatctggttccgcccgatgaagagcaagaggatcaacaaggtgtggtccccgaagcagctcaaggagcagttcctgaagtactaccaggagtacgaggccgacgtgaagatcaacgacatggtggaggcctacttcgacagcccgctgggcgagaactacgtgtgggtggactgcaggaagaagtacaagcagctggtgaaggagctggcctcaatcgccaagaccaccgaggccaacctgaaggaggacctggactgtgacctcgagtgcctcttccgcccgagcgagaagaagatgaagctctacggcagcaacaagtcctgggccatcatctccaacctgttcggcgagggcgacaaggaggaccgctccaagaagatcaagatcctgaccaaggctatccagatcctgaccgagagcaacccggagagctacgccgacgtgcagaaggccttcctcgccgccgccgacatcgatgaccctaagaagttccacacccaggagatctggggcaacggcagcccgggcaacatcgttaagatggccaggggcgatttcctcggcaaggagttcgactgcgagaagatcctcgagaagatcaacgacgttctcaaggagaagaccctggatttcgacctgaaggttaggctcagcttcaaggagtacctgatctcaaagatcggccactactaccagaactcatggtccgagatgatcaactcagccttcgccgacatcatctccaagaacacccgcaacgtgaacttcgctaaggagaaggttcagctgcagaagaccctcagcgagacctccaacgccaaggtggagctgctgaccgacttcttcaagagcgacttcttcctcggcgacgacaagttcgatatcgctccgcacaacctcggcggcgccaacggcatcaagttcttctacgatttctgcaagaagaacgaggaccagtacttcctcgaggagctgctcctcgaggccgccatcgaggagtccgtggccgaggccaagtccaagtccctgaaggagcctcataaggacctcctcaggtacgtgttcagcatcaggaaggagaccaccttcgaggagctgagggacgccgccaagtacatccagacccacaagaggatcaagaacatgtccgtgcacccgactgtgaagtcagacatcggcttcaacgtgacctccggctcagccctcgtgggccacgtggtgagcccgagcaagaaaattaacggccgcatcgccggcgagagcggcttcatctggatctgtatgaagctctgggagggcggcgacaagtggatcgagcatcacatcccgttcaccgacactcgcttctacgagcagatctacaaatataacccggacagcaagctggagccggtggtgctcaggaccaagcgctacggcgtggacctcaccaagttcaacctgccgcctatgaagaccgacctgaagcacgtggccccgaaggagaagaacaagcacaactacgtgaaggtgcagaggcgcctgcagcgcctcaaccacccggacgtgccgaacaccatctggccgaagtccaacatcggcttcaccatcaggaggaagaacggcaagtacatcctgaacgtggtgcataaactgccgaagaacaaggttaagaagtccgtgaagccgaagttcggcgacatcctgatcggcgtggaccagaaccagactactaaccacacctgctccatctacaaggtggttaagaagaacactaaggaggccctgctcgtgccggagtccgacttctacctcaagaagatcgagaccatcaaggttaccagcttcactaaggcccgctacaactccgagccgatcgatcagctgcattacgagggcatctccgtggacaacgaggtgttcaagaactggtgcaaggacagggagcagttcgtggactcactcagcatcaaggagttcaagaacgagttcaagcgcattaaaaacaagaacgagaacctctactcattcaacgccgactacctctggctcctcaagaggatcatctccggcaagctgaacaagaagaagttcgatgtgtccgtgttcgagaagtccatcaggaacgagatcctggccatgtgcagcaaggagggcctcggcccgctgagggtgagctccctctcctcaaacagcctgaaaagcattggcttcctgaagagcgccatctgcagcttcatctccatcgccctcaaccgcaagggcatcgaggacaagaccgacgtgcagaagaacaagatcgaccctgagctcttcgacctcatcggcaagatcgagcagaagcgcgtgaacaagcgcatggagaagacccgccgcaacgccgacttcatcctcaccatggccgtggactaccagaagtcctcccagaagaacgtgttcctcttctgcgagggcaacctggagaccgctaagaccggcaacagcaagaagcgcaactccgctaacgtggactggtgctcccgcaagctgttcgacttcctgaaggagaagagcctgaggcacggcatctacttccacgctgttaccccgcactacacttcacaccaggaccctttcgagtaccacccgagcaacaaggtgatgctcccgaggttcgccaagttcgataagaacaaccctatccaggactgggccgagaaaaaatatctcggcttcgccaacagcgacccggagagcggcaccgctctgtactataaaaaaggcgtggagaacttcttcgcccattaccagaagggcttcaaggagaaggtggagctggccgagatgaaaaacgtgctgaactccaacctcaagaacggcaacctcgagcacgtgttctgcccgatccgcggcggccgctactacctcagcacccacccggtgacctccgacgctaagccgttcgagttcaacggccgcaagtgctacatctgcgacagcgacgaggtggccgccaccaacatcatgctgatcggcctgttctacgtt

SEQ ID NO: 12 - DNA, 3162 bp, synthetic construct
  source 1..3162: mol_type = other DNA
atgagcagcgccatcaagagctacaagtccgtgctgcgcccgaacgagcgcaagaaccagctcctcaagagcaccatccagtgcctcgaggacggcagcgccttcttcttcaagatgctgcagggcctcttcggcggcatcaccccggagatcgtgaggttctcaaccgagcaggagaagcagcagcaggacatcgctctctggtgcgccgtgaactggttcaggccggtgtcccaggactccctcactcacaccatcgcctcagataacctggtggagaagttcgaggagtactacggcggcaccgcctccgacgccatcaagcagtacttcagcgcctccatcggcgagagctactactggaacgactgccgccagcagtactacgacctctgtcgcgagctgggcgtggaggtgtcagacctcacccacgacctcgagatcctctgccgcgagaagtgtctcgccgtggccaccgagagcaaccagaacaacagcatcatcagcgttctcttcggcaccggcgagaaggaggacaggtccgtgaagctcaggatcactaagaagatcctggaggccatctccaacctcaaggagatccctaagaacgttgccccgatccaggagatcatcctgaacgtggctaaggccaccaaggagaccttccgccaggtttacgccggcaacctgggcgccccgtccaccctggagaagttcatcgccaaggacggccagaaggagttcgatctcaagaagctgcagaccgacctgaagaaggtgatcaggggcaagtccaaggagcgcgactggtgttgccaggaggagctccgctcctacgtggagcagaacaccatccagtacgacctgtgggcctggggcgagatgttcaacaaggctcacaccgccctgaaaattaaaagcaccaggaactacaacttcgccaagcagaggctggagcagttcaaggagatccagagcctcaacaacctgctggtggtgaagaagctcaacgacttcttcgactccgagttcttctccggcgaggagacctacaccatctgcgtgcaccatctgggcggcaaggacctctcaaagctctacaaggcctgggaggacgacccggccgaccctgagaacgccatcgtggtgctgtgcgacgacctcaagaacaacttcaagaaggagccgatcaggaacatcctcaggtacatcttcaccatccgccaggagtgctcagcccaggacatcctggctgccgctaaatataaccagcagctggacaggtataaaagccagaaggccaacccgtccgttctgggcaaccagggcttcacttggaccaacgccgttatcctcccggagaaggcccagcgcaacgacaggccgaactccctggacctcaggatctggctctacctcaagctgaggcacccggacggcaggtggaagaagcaccacatcccgttctacgacacccgcttcttccaggagatctacgccgccggcaacagcccggtggatacctgccagttcaggaccccgaggttcggctaccatctcccgaagctgaccgaccagaccgccatccgcgtgaacaagaagcacgtgaaggccgccaagaccgaggctaggatcaggctcgctatccagcagggcaccctgcctgtgagcaacctcaagatcactgagatctccgccactatcaactccaagggccaggtgcgcatcccggttaagttcgacgtgggccgccagaagggcaccctgcagatcggcgaccgcttctgcggctacgaccagaaccagaccgccagccacgcttactccctctgggaggtggtgaaggagggccagtaccataaggagctcggctgcttcgtgaggttcatctcaagcggcgacatcgttagcatcaccgagaaccgcggcaaccagttcgaccagctgagctacgagggcctcgcctacccgcagtacgccgactggaggaagaaggccagcaagttcgtgtccctgtggcagatcaccaagaagaacaagaagaaggagatcgtgaccgttgaggccaaggagaagttcgatgccatctgcaagtaccagccgaggctgtacaagttcaacaaggagtacgcctacctgctcagggacatcgtgcgcggcaagagcctggtggagctgcagcagatcaggcaggagatcttccgcttcatcgagcaggactgcggcgtgacccgcctgggctccctctccctgtcaaccctcgagaccgtgaaggccgtgaagggcatcatctactcctacttctccaccgccctcaacgcctccaagaacaacccgatcagcgatgagcagaggaaggagttcgacccggagctgttcgctctcctcgagaagctggagctgatccgcacccgcaagaagaagcagaaggtggagcgcatcgccaacagcctgatccagacctgcctcgagaacaacatcaagttcatcaggggcgagggcgatctgtcaaccaccaacaacgccactaagaagaaggccaacagccgcagcatggactggctcgcccgcggcgtgttcaacaagatccgccagctcgccccgatgcacaacatcaccctgttcggctgcggctccctgtacacctcccaccaggacccgctcgtgcaccgcaacccggacaaggccatgaaatgccgctgggccgccatcccggtgaaggacatcggcgactgggtgctcaggaagctcagccagaacctgagggccaagaacatcggcaccggcgagtactaccaccagggcgtgaaggagttcctgtcacactacgagctccaggatctggaggaggagctgctcaagtggaggtccgaccgcaagagcaacatcccttgctgggtgctgcagaacaggctggccgagaagctcggcaacaaggaggccgtggtgtacatccctgttaggggcggccgcatctacttcgccactcataaagtggccaccggcgccgtgtccatcgtgttcgaccagaagcaggtgtgggtgtgcaacgccgaccacgtggccgctgctaacatcgccctgactgtgaagggcatcggcgagcagtcatccgacgaggagaacccggatggcagccgcattaaactccagctgaccagc

SEQ ID NO: 13 - DNA, 3237 bp, synthetic construct
  source 1..3237: mol_type = other DNA
atgccgaagcagaaggacctgccgtactcctgcctctcctacctgcagccgaacgagcgcaagctcaagctcctgaacaacacttacgaccacctcacccacggcagcaagatcatgttcgacaccctgatcgccctcatgggcggcatcaacccgaagatggacgtgatcagcgagaacaaggactccgagatcaagaacaacagggacccgcagaccatgtgcgccactatctggttccgcccgatgaagtccaagaggatcaacaaggtgtggagcccgaagcagctgaaggagcagttcctgaagtactaccaggagtacgaggccgacgtgaagatcaacgacatggtggaggcctacttcgactccccgctgggcgagaactacgtgtgggtggactgtaggaagaagtacaagcagctggtgaaggagctggcctccatcgccaagactaccgaggctaacctgaaggaggacctcgactgcgacctcgagtgcctcttccgcccgtccgagaagaagatgaagctgtacggcagcaacaagtcctgggccatcatctccaacctgttcggcgagggcgacaaggaggatcgcagcaagaagatcaagatcctcaccaaggccatccagatcctcaccgagagcaacccggagagctacgccgatgtgcagaaggctttcctcgccgccgccgacatcgacgacccgaagaagttccacacccaggagatctggggcaacggctccccgggcaacatcgtgaagatggccaggggcgacttcctcggcaaggagttcgactgcgagaagatcctggagaagatcaacgacgtgctgaaggagaagactctcgacttcgacctcaaggttcgcctgagcttcaaggagtacctcatctcaaagatcggccactactaccagaacagctggtccgagatgatcaacagcgccttcgccgacatcatcagcaagaacaccaggaacgttaacttcgccaaggagaaggtgcagctccagaagaccctgagcgagacctccaacgctaaggtggagctcctgactgatttcttcaagagcgacttcttcctcggcgacgacaagttcgacatcgccccgcacaacctgggcggcgctaacggcatcaagttcttctacgatttctgcaagaagaacgaggaccagtacttcctggaggagctgctcctggaggccgccatcgaggagagcgtggccgaggccaagtccaagtcactgaaggagccgcacaaggacctgctgcgctacgtgttctccatcaggaaggagaccaccttcgaggagctccgcgacgccgccaagtacatccagacccacaagaggatcaagaacatgtccgtgcaccctaccgtgaagtccgacatcggcttcaacgttacctccggcagcgccctcgtgggccatgtggtgagcccgtccaagaaaattaacggccgcatcgccggcgagagcggcttcatctggatctgcatgaagctctgggagggcggcgacaagtggatcgagcaccatatcccgttcaccgacacccgcttctacgagcagatctacaaatataaccctgacagcaagctggagccggtggtgctcaggaccaagcgctacggcgtggatctgactaagttcaacctcccgccgatgaagaccgacctcaagcacgttgcccctaaggagaagaacaagcacaactacgtgaaggtgcagcgcaggctccagcgcctgaaccacccggacgtgccgaacaccatctggccgaagtccaacatcggcttcaccatcaggcgcaagaacggcaagtacatcctcaacgtggtgcataaactcccgaagaacaaggtgaagaagtccgtgaagccgaagttcggcgatatcctgatcggcgttgaccagaaccagaccaccaaccatacctgctccatctacaaggttgtgaagaagaacactaaggaggctctgctcgtgccggagtccgatttctacctcaagaagatcgagaccatcaaggtgacctccttcaccaaggccaggtacaacagcgagccgatcgaccagctccactacgagggcatcagcgttgataacgaggtgttcaagaactggtgcaaggaccgcgagcagttcgtggacagcctgagcatcaaggagttcaagaacgagttcaagaggattaaaaacaagaacgagaacctctacagcttcaacgccgactacctctggctcctcaagaggatcatcagcggcaagctgaacaagaagaagttcgacgtgtcagtgttcgagaagagcatccgcaacgagatcctcgccatgtgcagcaaggagggcctcggcccgctgcgcgtgtccagcctgtcatcaaactccctcaaaagcattggcttcctgaagtccgccatctgctccttcatctcaatcgctctgaacaggaagggcatcgaggacaagaccgacgtgcagaagaacaagatcgaccctgagctgttcgacctgatcggcaagatcgagcagaagcgcgtgaacaagaggatggagaagacccgcaggaacgccgacttcatcctcaccatggctgtggattaccagaagtcaagccagaagaacgttttcctgttctgcgagggcaacctcgagaccgccaagaccggcaactccaagaagcgcaactcagccaacgtggactggtgctccaggaagctgttcgatttcctgaaggagaagagcctgaggcacggcatctacttccacgccgtgaccccgcactacaccagccaccaggacccgttcgagtaccaccctagcaacaaggtgatgctgccgcgcttcgccaagttcgacaagaacaacccgatccaggactgggccgagaaaaaatatctgggcttcgccaactcagacccggagagcggcaccgccctctactataaaaaaggcgtggagaacttcttcgcccactaccagaagggcttcaaggagaaggtggagctggccgagatgaaaaacgtgctgaactccaacctcaagaacggcaacctcgagcacgtgttctgcccgatcaggggcggccgctactacctctccacccatcctgtgacctcagacgccaagccgttcgagttcaacggcaggaagtgttacatctgtgactccgacgaggtggctgccaccaacatcatgctgatcggcctcttctacgtt

SEQ ID NO: 14 - DNA, 3237 bp, synthetic construct
  source 1..3237: mol_type = other DNA
atgcctaagcagaaggacctgccttacagctgtctcagctacctgcagccgaacgagcgcaagctcaagctgctcaacaacacctacgaccacctgacccacggctcaaagatcatgttcgataccctcatcgccctcatgggcggcatcaacccgaagatggacgtgatcagcgagaacaaggacagcgagatcaagaacaaccgcgacccgcagaccatgtgcgccaccatctggttccgcccgatgaagagcaagaggatcaacaaggtgtggagcccgaagcagctcaaggagcagttcctcaagtactaccaggagtacgaggccgatgtgaagatcaacgacatggttgaggcttacttcgacagcccgctcggcgagaactacgtgtgggtggactgcaggaagaagtacaagcagctggtgaaggagctggccagcatcgctaagaccaccgaggccaacctgaaggaggacctggactgcgatctcgagtgcctgttccgcccgtcagagaagaagatgaagctgtacggcagcaacaagtcatgggccatcatctccaacctcttcggcgagggcgacaaggaggacaggtccaagaagatcaagatcctgaccaaggccatccagatcctcaccgagtcaaacccggagagctacgccgacgttcagaaggccttcctggccgccgctgacatcgacgacccgaagaagttccacacccaggagatctggggcaacggcagcccgggcaacatcgtgaagatggcccgcggcgacttcctcggcaaggagttcgattgcgagaagatcctcgagaagatcaacgacgtgctgaaggagaagactctggacttcgacctgaaggtgaggctctccttcaaggagtacctgatcagcaagatcggccactactaccagaactcctggtccgagatgatcaactccgccttcgctgatatcatcagcaagaacactaggaacgtgaacttcgctaaggagaaggtgcagctccagaagaccctgtcagagacctccaacgccaaggtggagctgctgaccgacttcttcaagtccgatttcttcctcggcgacgacaagttcgacatcgccccgcataacctgggcggcgccaacggcatcaagttcttctacgatttctgcaagaagaacgaggaccagtacttcctcgaggagctgctcctggaggccgccatcgaggagagcgtggccgaggccaagtccaagagcctcaaggagccgcacaaggacctcctcaggtacgtgttcagcatccgcaaggagaccaccttcgaggagctgagggacgccgctaagtacatccagacccacaagaggatcaagaacatgtccgtgcatccgaccgtgaagagcgacatcggcttcaacgtgacctccggcagcgccctggttggccacgtggtgagcccgtccaagaaaattaacggccgcatcgccggcgagtccggcttcatctggatctgcatgaagctctgggagggcggcgacaagtggatcgagcaccacatcccgttcaccgacactaggttctacgagcagatctacaaatataacccggactccaagctggagccggtggtgctccgcaccaagcgctacggcgtggacctcaccaagttcaacctgccgccgatgaagaccgacctcaagcacgtggcccctaaggagaagaacaagcacaactacgtgaaggtgcagcgcaggctgcagcgcctcaaccacccggacgtgccgaacaccatctggccgaagtccaacatcggcttcaccatcaggcgcaagaacggcaagtacatcctgaacgttgtgcataaactgcctaagaacaaggtgaagaagtccgtgaagccgaagttcggcgacatcctcatcggcgtggatcagaaccagaccaccaaccacacctgctccatctacaaggtggtgaagaagaacaccaaggaggccctgctggtgccggagtccgacttctacctcaagaagatcgagactatcaaggttacctcattcactaaggcccgctacaacagcgagccgatcgaccagctccattacgagggcatcagcgtggacaacgaggtgttcaagaactggtgcaaggaccgcgagcagttcgtggacagcctctccatcaaggagttcaagaacgagttcaagaggattaaaaacaagaacgagaacctctactccttcaacgccgactacctgtggctgctcaagcgcatcatctccggcaagctgaacaagaagaagttcgacgtttccgtgttcgagaagtcaatcaggaacgagatcctggccatgtgctccaaggagggcctgggcccgctcagggtgagctccctctcctccaacagcctgaaaagcattggcttcctcaagagcgccatctgttccttcatctcaatcgccctgaacaggaagggcatcgaggacaagaccgacgttcagaagaacaagatcgacccggagctgttcgatctcatcggcaagatcgagcagaagcgcgtgaacaagcgcatggagaagactaggaggaacgccgacttcatcctcaccatggccgttgactaccagaagagctcccagaagaacgtgttcctgttctgcgagggcaacctggagaccgccaagactggcaacagcaagaagcgcaactcagccaacgtggactggtgttcccgcaagctgttcgacttcctgaaggagaagtccctcaggcacggcatctacttccacgccgtgacccctcattacaccagccaccaggaccctttcgagtaccacccgagcaacaaggtgatgctcccgaggttcgccaagttcgataagaacaacccgatccaggactgggccgagaaaaaatatctcggcttcgccaacagcgacccggagtccggcaccgctctctactataaaaaaggcgtggagaacttcttcgcccactaccagaagggcttcaaggagaaggtggagctcgccgagatgaaaaacgtgctcaactccaacctgaagaacggcaacctggagcacgtgttctgcccgatccgcggcggccgctactacctcagcacccacccggtgaccagcgacgccaagccgttcgagttcaacggcaggaagtgctacatctgcgactcagacgaggttgctgccaccaacatcatgctcatcggcctgttctacgtg

SEQ ID NO: 15 - DNA, 3237 bp, synthetic construct
  source 1..3237: mol_type = other DNA
atgccgaagcagaaggacctgccttacagctgtctctcctacctgcagccgaacgagcgcaagctcaagctcctcaacaacacttacgaccacctcacccacggcagcaagatcatgttcgataccctcatcgctctgatgggcggcatcaacccgaagatggatgtgatcagcgagaacaaggacagcgagatcaagaacaaccgcgaccctcagaccatgtgtgccaccatctggttccgcccgatgaagagcaagaggatcaacaaggtgtggtccccgaagcagctgaaggagcagttcctgaagtactaccaggagtacgaggccgacgtgaagatcaacgacatggtggaggcttacttcgacagcccgctcggcgagaactacgtgtgggtggactgccgcaagaagtacaagcagctcgtgaaggagctggccagcatcgccaagactactgaggccaacctgaaggaggacctggactgcgacctcgagtgcctcttccgcccttccgagaagaagatgaagctctacggcagcaacaagtcctgggccatcatctccaacctgttcggcgagggcgacaaggaggacaggtccaagaagatcaagatcctgaccaaggccatccagatcctcaccgagtccaacccggagtcctacgccgatgtgcagaaggccttcctggccgccgctgacatcgacgacccgaagaagttccacacccaggagatctggggcaacggctccccgggcaacatcgttaagatggcccgcggcgatttcctcggcaaggagttcgactgcgagaagatcctcgagaagatcaacgacgtgctcaaggagaagaccctcgacttcgacctgaaggtgaggctctccttcaaggagtacctcatcagcaagatcggccactactaccagaactcctggtccgagatgatcaacagcgccttcgccgacatcatctccaagaacactcgcaacgtgaacttcgccaaggagaaggttcagctgcagaagaccctctccgagacctccaacgccaaggttgagctgctcaccgacttcttcaagagcgacttcttcctcggcgacgacaagttcgacatcgccccgcacaacctgggcggcgccaacggcatcaagttcttctacgatttctgcaagaagaacgaggaccagtacttcctggaggagctcctcctggaggccgccatcgaggagtccgtggccgaggccaagtccaagtcactcaaggagccgcataaggacctgctgcgctacgtgttctccatcaggaaggagaccaccttcgaggagctgcgcgacgccgccaagtacatccagacccacaagaggatcaagaacatgtccgtgcatccgaccgtgaagagcgacatcggcttcaacgtgacctcaggctcagccctggtgggccatgtggtgagcccgtcaaagaaaattaacggcaggatcgctggcgagtccggcttcatctggatctgcatgaagctctgggagggcggcgacaagtggatcgagcaccacatcccgttcaccgacacccgcttctacgagcagatctacaaatataacccggacagcaagctggagccggttgtgctgaggaccaagaggtacggcgtggacctgaccaagttcaacctcccgccgatgaagaccgatctgaagcacgtggccccgaaggagaagaacaagcacaactacgtgaaggtgcagcgcaggctccagaggctcaaccacccggacgttcctaacaccatctggcctaagagcaacatcggcttcaccatcaggaggaagaacggcaagtacatcctcaacgtggtgcataaactcccgaagaacaaggtgaagaagtcagtgaagccgaagttcggcgacatcctgatcggcgtggatcagaaccagaccaccaaccacacctgctccatctacaaggtggtgaagaagaacaccaaggaggccctcctggtgccggagagcgacttctacctcaagaagatcgagaccatcaaggttaccagcttcaccaaggctcgctacaacagcgagccgatcgatcagctgcactacgagggcatctcagtggacaacgaggtgttcaagaactggtgcaaggaccgcgagcagttcgttgacagcctctccatcaaggagttcaagaacgagttcaagaggattaaaaacaagaacgagaacctctactccttcaacgctgactacctgtggctcctcaagcgcatcatctcaggcaagctcaacaagaagaagttcgacgtgagcgtgttcgagaagtccatccgcaacgagatcctcgccatgtgcagcaaggagggcctgggcccgctgagggtgtcaagcctcagctccaacagcctgaaaagcattggcttcctgaagtccgctatctgttccttcatctccatcgccctgaacaggaagggcatcgaggataagactgacgtgcagaagaacaagatcgacccggagctgttcgacctgatcggcaagatcgagcagaagcgcgtgaacaagcgcatggagaagacccgccgcaacgccgacttcatcctcactatggccgtggattaccagaagagcagccagaagaacgtgttcctgttctgcgagggcaacctcgagaccgccaagactggcaacagcaagaagcgcaacagcgccaacgtggactggtgctccaggaagctgttcgacttcctgaaggagaagtccctgaggcacggcatctacttccatgccgtgaccccgcactacaccagccaccaggacccgttcgagtaccacccgtccaacaaggtgatgctcccgaggttcgccaagttcgacaagaacaacccgatccaggactgggctgagaaaaaatatctgggcttcgccaacagcgaccctgagtcaggcaccgccctgtactataaaaaaggcgttgagaacttcttcgcccactaccagaagggcttcaaggagaaggtggagctcgccgagatgaaaaacgtgctgaactcaaacctgaagaacggcaacctcgagcacgtgttctgcccgatcaggggcggccgctactacctgagcacccacccggtgacctccgacgccaagccgttcgagttcaacggcaggaagtgctacatctgcgacagcgacgaggttgccgccaccaacatcatgctgatcggcctcttctacgtg

SEQ ID NO: 16 - DNA, 3237 bp, synthetic construct
  source 1..3237: mol_type = other DNA
atgccgaagcagaaggacctgccgtacagctgcctgagctacctccagccgaacgagaggaagctgaagctgctcaacaacacttacgaccatctcacccacggctcaaagatcatgttcgacaccctgatcgccctcatgggcggcatcaacccgaagatggacgtgatctccgagaacaaggacagcgagatcaagaacaacagggatcctcagaccatgtgcgccactatctggttcaggccgatgaagagcaagaggatcaacaaggtgtggtccccgaagcagctcaaggagcagttcctcaagtactaccaggagtacgaggccgatgtgaagatcaacgacatggtggaggcctacttcgacagcccgctgggcgagaactacgtgtgggttgactgcaggaagaagtacaagcagctggtgaaggagctggcctcaatcgctaagaccaccgaggccaacctgaaggaggatctcgactgcgacctggagtgtctcttccgcccgtccgagaagaagatgaagctctacggctcaaacaagagctgggccatcatcagcaacctcttcggcgagggcgacaaggaggacaggtccaagaagatcaagatcctgaccaaggccatccagatcctgaccgagtccaacccggagtcctacgctgacgtgcagaaggccttcctggccgccgccgacatcgacgatccgaagaagttccacacccaggagatctggggcaacggcagcccgggcaacatcgtgaagatggcccgcggcgatttcctgggcaaggagttcgactgcgagaagatcctggagaagatcaacgacgtgctgaaggagaagaccctcgacttcgacctgaaggttcgcctgtccttcaaggagtacctgatcagcaagatcggccactactaccagaactcatggtcagagatgatcaacagcgctttcgccgatatcatctcaaagaacacccgcaacgtgaacttcgccaaggagaaggttcagctccagaagactctgtccgagacctccaacgccaaggtggagctcctcaccgacttcttcaagtccgacttcttcctgggcgacgacaagttcgatatcgcccctcacaacctcggcggcgccaacggcatcaagttcttctacgacttctgcaagaagaacgaggaccagtacttcctggaggagctcctcctggaggccgccatcgaggagtccgtggctgaggctaagagcaagagcctgaaggagcctcacaaggacctcctccgctacgtgttctccatccgcaaggagaccaccttcgaggagctgagggatgccgctaagtacatccagacccacaagaggatcaagaacatgtccgtgcacccgaccgttaagagcgacatcggcttcaacgtgacctcaggcagcgccctcgttggccacgtggtgtccccgagcaagaaaattaacggccgcatcgccggcgagtccggcttcatctggatctgcatgaagctctgggagggcggcgacaagtggatcgagcaccacatcccgttcaccgacacccgcttctacgagcagatctacaaatataacccggactccaagctggagccggtggtgctgaggactaagcgctacggcgtggacctgaccaagttcaacctgccgcctatgaagaccgacctgaagcacgtggcccctaaggagaagaacaagcataactacgtgaaggtgcagcgccgcctgcagcgcctcaaccacccggatgtgccgaacaccatctggccgaagtccaacatcggcttcaccatccgccgcaagaacggcaagtacatcctcaacgtggtgcataaactgccgaagaacaaggtgaagaagtccgtgaagccgaagttcggcgacatcctcatcggcgttgaccagaaccagaccaccaaccacacctgctccatctacaaggttgtgaagaagaacactaaggaggccctcctcgtgccggagtccgacttctacctgaagaagatcgagaccatcaaggtgaccagcttcaccaaggccaggtacaacagcgagcctatcgaccagctccactacgagggcatcagcgttgacaacgaggtgttcaagaactggtgcaaggatcgcgagcagttcgtggactcactgtcaatcaaggagttcaagaacgagttcaagcgcattaaaaacaagaacgagaacctctactccttcaacgccgactacctctggctgctgaagcgcatcatcagcggcaagctgaacaagaagaagttcgacgtgtccgtgttcgagaagagcatcaggaacgagatcctggccatgtgcagcaaggagggcctgggcccgctcagggtgtcctccctgtccagcaacagcctcaaaagcattggcttcctcaagagcgccatctgcagcttcatcagcatcgccctcaacaggaagggcatcgaggacaagaccgacgtgcagaagaacaagatcgacccggagctcttcgacctcatcggcaagatcgagcagaagagggtgaacaagcgcatggagaagaccaggcgcaacgccgacttcatcctcactatggccgtggactaccagaagtcaagccagaagaacgtgttcctgttctgcgagggcaacctcgagaccgccaagaccggcaacagcaagaagaggaacagcgccaacgtggactggtgttccaggaagctgttcgacttcctcaaggagaagtccctgcgccatggcatctacttccacgccgtgaccccgcactacacctcccaccaggacccgttcgagtaccacccgtccaacaaggtgatgctcccgaggttcgccaagttcgacaagaacaacccgatccaggactgggccgagaaaaaatatctcggcttcgccaacagcgacccggagagcggcaccgctctctactataaaaaaggcgtggagaacttcttcgcccattaccagaagggcttcaaggagaaggtggagctcgccgagatgaaaaacgtgctgaactccaacctcaagaacggcaacctggagcacgtgttctgcccgatcaggggcggccgctactacctctccacccacccggttactagcgacgccaagccgttcgagttcaacggcaggaagtgctacatctgtgactccgacgaggtggccgctaccaacatcatgctcatcggcctgttctacgtg

SEQ ID NO: 17 - DNA, 3237 bp, synthetic construct
  source 1..3237: mol_type = other DNA
atgccgaagcagaaggacctcccgtactcctgtctgagctacctccagccgaacgagaggaagctcaagctcctcaacaacacctacgaccacctgacccacggcagcaagatcatgttcgacaccctgatcgccctcatgggcggcatcaaccctaagatggacgtgatctccgagaacaaggactcagagatcaagaacaacagggacccgcagaccatgtgcgccaccatctggttcaggccgatgaagagcaagcgcatcaacaaggtgtggtccccgaagcagctcaaggagcagttcctgaagtactaccaggagtacgaggccgacgtgaagatcaacgacatggtggaggcttacttcgactccccgctcggcgagaactacgtgtgggtggactgcaggaagaagtacaagcagctggttaaggagctggcctccatcgccaagaccaccgaggccaacctgaaggaggacctggactgcgatctcgagtgcctcttccgcccgtccgagaagaagatgaagctgtacggcagcaacaagagctgggccatcatcagcaacctgttcggcgagggcgacaaggaggacaggagcaagaagatcaagatcctcactaaggctatccagatcctgaccgagagcaaccctgagtcctacgccgacgtgcagaaggccttcctcgctgccgccgacatcgacgacccgaagaagttccatacccaggagatctggggcaacggctcccctggcaacatcgtgaagatggctcgcggcgatttcctcggcaaggagttcgactgcgagaagatcctggagaagatcaacgacgtgctcaaggagaagaccctggatttcgacctgaaggttcgcctgtcattcaaggagtacctgatctcaaagatcggccactactaccagaacagctggagcgagatgatcaacagcgccttcgccgacatcatcagcaagaacacccgcaacgtgaacttcgctaaggagaaggtgcagctgcagaagaccctctccgagacttcaaacgccaaggtggagctcctcaccgacttcttcaagtccgacttcttcctgggcgatgacaagttcgacatcgccccgcacaacctgggcggcgccaacggcatcaagttcttctacgacttctgcaagaagaacgaggatcagtacttcctggaggagctgctgctggaggccgccatcgaggagtccgtggccgaggccaagagcaagtccctcaaggagccgcacaaggacctcctgaggtacgtgttcagcatcaggaaggagaccaccttcgaggagctccgcgacgccgccaagtacatccagacccacaagaggatcaagaacatgtccgtgcaccctaccgtgaagtcagacatcggcttcaacgtgaccagcggcagcgccctggttggccacgtggtgagcccgtccaagaaaattaacggcaggatcgccggcgagtccggcttcatctggatctgcatgaagctctgggagggcggcgacaagtggatcgagcatcatatcccgttcaccgataccaggttctacgagcagatctacaaatataaccctgactccaagctcgagccggtggtgctgcgcactaagcgctacggcgtggacctcaccaagttcaacctcccgcctatgaagaccgatctgaagcacgtggccccgaaggagaagaacaagcacaactacgttaaggtgcagaggaggctgcagaggctcaaccatccggacgtgccgaacaccatctggccgaagagcaacatcggcttcaccatccgcaggaagaacggcaagtacatcctgaacgtggttcataaactcccgaagaacaaggtgaagaagagcgtgaagccgaagttcggcgacatcctcatcggcgttgaccagaaccagaccactaaccacacctgttccatctacaaggtggtgaagaagaacaccaaggaggccctcctggttccggagagcgacttctacctgaagaagatcgagactatcaaggtgactagcttcaccaaggcccgctacaactccgagccgatcgaccagctgcactacgagggcatcagcgtggataacgaggtgttcaagaactggtgcaaggaccgcgagcagttcgtggattccctgtcaatcaaggagttcaagaacgagttcaagaggattaaaaacaagaacgagaacctctactcattcaacgccgactacctgtggctcctcaagaggatcatctcaggcaagctgaacaagaagaagttcgacgtgagcgtgttcgagaagagcatccgcaacgagatcctcgccatgtgctccaaggagggcctcggcccgctccgcgtgagcagcctgtcctccaacagcctgaaaagcattggcttcctgaagtccgccatctgctcattcatctccatcgccctcaaccgcaagggcatcgaggacaagaccgacgtgcagaagaacaagatcgacccggagctcttcgacctcatcggcaagatcgagcagaagcgcgtgaacaagaggatggagaagacccgcaggaacgccgacttcatcctcaccatggccgttgactaccagaagtcctcccagaagaacgtgttcctgttctgcgagggcaacctggagaccgctaagaccggcaactccaagaagaggaacagcgccaacgttgactggtgctcccgcaagctgttcgacttcctgaaggagaagtccctccgccacggcatctacttccacgccgtgaccccgcactacacttcccaccaggatccgttcgagtaccacccgtccaacaaggtgatgctcccgcgcttcgccaagttcgacaagaacaacccgatccaggactgggccgagaaaaaatatctcggcttcgccaacagcgacccggagtcaggcaccgctctgtactataaaaaaggcgtggagaacttcttcgcccactaccagaagggcttcaaggagaaggtggagctcgccgagatgaaaaacgtgctcaacagcaacctgaagaacggcaacctcgagcacgtgttctgcccgatccgcggcggcaggtactacctgagcacccacccggtgaccagcgacgctaagccgttcgagttcaacggccgcaagtgttacatctgcgactccgacgaggtggccgccaccaacatcatgctcatcggcctgttctacgtg

SEQ ID NO: 18 - DNA, 3237 bp, synthetic construct
  source 1..3237: mol_type = other DNA
atgccgaagcagaaggacctgccgtactcctgcctgagctacctgcagccgaacgagaggaagctcaagctgctcaacaacacctacgaccacctcacccacggctccaagatcatgttcgacaccctgatcgccctgatgggcggcatcaaccctaagatggacgtgatcagcgagaacaaggacagcgagatcaagaacaacagggatccgcagaccatgtgcgccaccatctggttcaggccgatgaagagcaagcgcatcaacaaggtgtggagccctaagcagctgaaggagcagttcctcaagtactaccaggagtacgaggctgacgtgaagatcaacgacatggtggaggcctacttcgattcaccgctcggcgagaactacgtgtgggtggactgccgcaagaagtacaagcagctggtgaaggagctcgccagcatcgccaagaccaccgaggccaacctgaaggaggacctggactgcgacctggagtgcctcttccgcccgtcagagaagaagatgaagctgtacggctccaacaagagctgggctatcatcagcaacctcttcggcgagggcgacaaggaggataggtccaagaagatcaagatcctgaccaaggccatccagatcctgactgagagcaacccggagtcctacgccgacgtgcagaaggccttcctcgccgccgccgacatcgacgaccctaagaagttccacacccaggagatctggggcaacggctcccctggcaacatcgtgaagatggcccgcggcgacttcctcggcaaggagttcgactgtgagaagatcctcgagaagatcaacgacgtgctcaaggagaagaccctggacttcgacctcaaggtgcgcctgtccttcaaggagtacctgatctcaaagatcggccactactaccagaactcctggagcgagatgatcaactccgccttcgccgacatcatctccaagaacacccgcaacgttaacttcgctaaggagaaggtgcagctgcagaagaccctctccgagacctccaacgccaaggtggagctcctgactgacttcttcaagtccgatttcttcctcggcgacgacaagttcgacatcgccccgcacaacctcggcggcgccaacggcatcaagttcttctacgacttctgcaagaagaacgaggaccagtacttcctggaggagctcctgctggaggctgccatcgaggagtcagtggccgaggccaagagcaagtcactcaaggagccgcataaggacctcctcaggtacgttttcagcatcaggaaggagaccaccttcgaggagctgagggacgccgccaagtacatccagacccacaagcgcatcaagaacatgtccgtgcacccgaccgtgaagtccgacatcggcttcaacgtgactagcggcagcgccctcgtgggccacgtggtgtccccgagcaagaaaattaacggcaggatcgccggcgagtccggcttcatctggatctgcatgaagctgtgggagggcggcgacaagtggatcgagcaccacatcccgttcaccgacactcgcttctacgagcagatctacaaatataacccggattccaagctcgagccggtggtgctcaggaccaagaggtacggcgttgacctcactaagttcaacctgccgccgatgaagaccgacctcaagcatgttgccccgaaggagaagaacaagcacaactacgtgaaggtgcagaggaggctccagcgcctgaaccacccggacgtgccgaacaccatctggcctaagtcaaacatcggcttcaccatccgcaggaagaacggcaagtacatcctcaacgtggtgcataaactgccgaagaacaaggtgaagaagtccgtgaagccgaagttcggcgacatcctcatcggcgtggaccagaaccagaccaccaaccacacctgcagcatctacaaggttgtgaagaagaacaccaaggaggccctgctcgtgccggagtccgacttctacctgaagaagatcgagaccatcaaggtgacctccttcaccaaggcccgctacaacagcgagccgatcgaccagctgcactacgagggcatcagcgtggataacgaggtgttcaagaactggtgcaaggaccgcgagcagttcgtggactccctctccatcaaggagttcaagaacgagttcaagcgcattaaaaacaagaacgagaacctgtactccttcaacgccgattacctgtggctgctcaagaggatcatctccggcaagctcaacaagaagaagttcgacgtgagcgtgttcgagaagtccatccgcaacgagatcctcgccatgtgctcaaaggagggcctcggcccgctgcgcgtgagctccctcagcagcaactcactcaaaagcattggcttcctgaagagcgccatctgcagcttcatctccatcgccctgaacaggaagggcatcgaggacaagaccgacgttcagaagaacaagatcgacccggagctgttcgacctgatcggcaagatcgagcagaagcgcgtgaacaagaggatggagaagacccgccgcaacgccgacttcatcctgaccatggctgtggactaccagaagagctcccagaagaacgtgttcctgttctgcgagggcaacctcgagaccgctaagactggcaacagcaagaagaggaacagcgccaacgtggactggtgtagccgcaagctcttcgatttcctcaaggagaagagcctcaggcacggcatctacttccacgccgttaccccgcactacacttcacaccaggacccgttcgagtaccacccgtcaaacaaggttatgctcccgaggttcgccaagttcgacaagaacaacccgatccaggactgggccgagaaaaaatatctgggcttcgccaactccgaccctgagagcggcaccgccctgtactataaaaaaggcgtggagaacttcttcgctcattaccagaagggcttcaaggagaaggtggagctcgccgagatgaaaaacgtgctgaacagcaacctcaagaacggcaacctggagcacgttttctgtccgatcaggggcggccgctactacctgtccacccatccggtgaccagcgatgccaagccgttcgagttcaacggccgcaagtgctacatctgcgattccgacgaggtggctgccaccaacatcatgctcatcggcctgttctacgtg

SEQ ID NO: 19 - DNA, 3237 bp, synthetic construct
  source 1..3237: mol_type = other DNA
atgccgaagcagaaggacctcccttacagctgcctgtcctacctccagccgaacgagcgcaagctgaagctcctgaacaacacctacgaccacctcacccacggctccaagatcatgttcgacactctcatcgccctcatgggcggcatcaacccgaagatggacgtgatcagcgagaacaaggactccgagatcaagaacaaccgcgacccgcagaccatgtgcgccaccatctggttcaggccgatgaagagcaagcgcatcaacaaggtgtggagcccgaagcagctcaaggagcagttcctcaagtactaccaggagtacgaggccgacgtgaagatcaacgacatggtggaggcctacttcgacagcccgctcggcgagaactacgtgtgggtggactgcaggaagaagtacaagcagctggttaaggagctcgcctccatcgccaagaccaccgaggccaacctgaaggaggacctggactgcgacctggagtgcctcttcaggccgagcgagaagaagatgaagctctacggcagcaacaagtcatgggccatcatctccaacctcttcggcgagggcgataaggaggaccgctccaagaagatcaagatcctcactaaggccatccagatcctcaccgagagcaaccctgagagctacgccgacgtgcagaaggccttcctggccgccgccgacatcgacgatccgaagaagttccacacccaggagatctggggcaacggctccccgggcaacatcgtgaagatggcccgcggcgacttcctcggcaaggagttcgactgcgagaagatcctggagaagatcaacgacgtgctcaaggagaagaccctggacttcgacctgaaggtgcgcctgagcttcaaggagtacctgatctccaagatcggccattactaccagaactcctggtccgagatgatcaacagcgctttcgctgacatcatcagcaagaacacccgcaacgtgaacttcgccaaggagaaggtgcagctgcagaagaccctcagcgagacctcaaacgccaaggttgagctgctcaccgacttcttcaagagcgacttcttcctcggcgatgacaagttcgacatcgccccgcacaacctgggcggcgccaacggcatcaagttcttctacgacttctgcaagaagaacgaggaccagtacttcctggaggagctcctgctcgaggctgccatcgaggagtccgtggctgaggccaagagcaagtcactgaaggagccgcacaaggacctgctccgctacgtgttctccatccgcaaggagaccaccttcgaggagctgagggacgccgccaagtacatccagacccacaagaggatcaagaacatgtccgtgcacccgaccgtgaagtccgacatcggcttcaacgtgaccagcggcagcgctctcgtgggccacgtggtgtcaccgagcaagaaaattaacggccgcatcgccggcgagtccggcttcatctggatctgcatgaagctctgggagggcggcgacaagtggatcgagcaccatatcccgttcaccgatacccgcttctacgagcagatctacaaatataacccggacagcaagctggagcctgttgtgctccgcaccaagaggtacggcgtggacctgaccaagttcaacctcccgccgatgaagactgatctgaagcacgttgccccgaaggagaagaacaagcacaactacgtgaaggtgcagaggaggctccagcgcctcaaccacccggacgtgccgaacaccatctggccgaagtccaacatcggcttcaccatccgcaggaagaacggcaagtacatcctcaacgtggtgcataaactgccgaagaacaaggtgaagaagtccgtgaagccgaagttcggcgacatcctgatcggcgttgatcagaaccagaccaccaaccacacctgcagcatctacaaggtggtgaagaagaacaccaaggaggctctgctcgtgccggagtccgacttctacctcaagaagatcgagaccatcaaggtgacctccttcactaaggccaggtacaactccgagccgatcgaccagctccactacgagggcatctccgtggacaacgaggtgttcaagaactggtgtaaggacagggagcagttcgttgatagcctctcaatcaaggagttcaagaacgagttcaagaggattaaaaacaagaacgagaacctctacagcttcaacgccgactacctgtggctgctgaagaggatcatctccggcaagctgaacaagaagaagttcgacgtgtcagtgttcgagaagtccatccgcaacgagatcctcgccatgtgcagcaaggagggcctgggccctctgcgcgtgagctcactgagctccaacagcctcaaaagcattggcttcctgaagtccgccatctgttccttcatcagcatcgctctgaacaggaagggcatcgaggacaagaccgacgttcagaagaacaagatcgacccggagctgttcgacctcatcggcaagatcgagcagaagcgcgtgaacaagaggatggagaagacccgccgcaacgccgatttcatcctcactatggccgtggactaccagaagtcctcccagaagaacgtgttcctgttctgtgagggcaacctggagactgccaagaccggcaacagcaagaagaggaacagcgccaacgtggactggtgctcccgcaagctgttcgacttcctgaaggagaagtccctgaggcacggcatctacttccacgccgtgacccctcattacacctcacaccaggacccgttcgagtaccacccgagcaacaaggtgatgctcccgcgcttcgccaagttcgacaagaacaacccgatccaggactgggccgagaaaaaatatctcggcttcgccaactccgatcctgagtcaggcaccgccctgtactataaaaaaggcgttgagaacttcttcgcccactaccagaagggcttcaaggagaaggtggagctcgccgagatgaaaaacgtgctgaactcaaacctgaagaacggcaacctcgagcacgtgttctgcccgatcaggggcggcaggtactacctcagcacccatccggtgactagcgacgccaagccgttcgagttcaacggcaggaagtgctacatctgcgactccgatgaggtggctgccaccaacatcatgctcatcggcctgttctacgtt

SEQ ID NO: 20 - DNA, 3237 bp, synthetic construct
  source 1..3237: mol_type = other DNA
atgccgaagcagaaggacctgccgtacagctgcctgagctacctccagccgaacgagaggaagctcaagctgctgaacaacacctacgaccacctcactcacggcagcaagatcatgttcgacaccctcatcgccctgatgggcggcatcaaccctaagatggatgtgatcagcgagaacaaggattccgagatcaagaacaaccgcgacccgcagaccatgtgcgccaccatctggttccgcccgatgaagtccaagcgcatcaacaaggtgtggagcccgaagcagctgaaggagcagttcctgaagtactaccaggagtacgaggccgacgtgaagatcaacgacatggtggaggcctacttcgacagcccgctgggcgagaactacgtgtgggtggactgccgcaagaagtacaagcagctcgtgaaggagctcgcctcaatcgccaagaccactgaggccaacctcaaggaggacctcgactgtgacctcgagtgcctgttcaggccgtccgagaagaagatgaagctctacggctccaacaagtcctgggccatcatctccaacctgttcggcgagggcgacaaggaggacaggtcaaagaagatcaagatcctgaccaaggctatccagatcctcactgagagcaacccggagagctacgccgacgttcagaaggctttcctcgccgccgccgacatcgacgacccgaagaagttccacacccaggagatctggggcaacggctccccgggcaacatcgtgaagatggcccgcggcgacttcctgggcaaggagttcgactgcgagaagatcctcgagaagatcaacgacgtgctcaaggagaagaccctcgacttcgacctgaaggtgaggctgagcttcaaggagtacctcatctccaagatcggccactactaccagaactcatggtccgagatgatcaactcagccttcgccgacatcatctccaagaacactaggaacgtgaacttcgccaaggagaaggttcagctgcagaagaccctcagcgagaccagcaacgccaaggtggagctgctcaccgacttcttcaagagcgacttcttcctgggcgacgacaagttcgacatcgcccctcacaacctcggcggcgccaacggcatcaagttcttctacgacttctgcaagaagaacgaggaccagtacttcctggaggagctgctcctggaggccgccatcgaggagtccgtggctgaggccaagtccaagagcctcaaggagccgcacaaggatctcctgaggtacgtgttctccatcaggaaggagaccaccttcgaggagctccgcgacgccgccaagtacatccagacccacaagcgcatcaagaacatgagcgtgcaccctaccgtgaagagcgacatcggcttcaacgttactagcggcagcgccctcgtgggccacgtggtgtccccgtcaaagaaaattaacggcaggatcgccggcgagagcggcttcatctggatctgcatgaagctgtgggagggcggcgacaagtggatcgagcaccatatccctttcaccgataccaggttctacgagcagatctacaaatataacccggactccaagctggagccggtggtgctccgcaccaagcgctacggcgtggacctgaccaagttcaacctcccgccgatgaagaccgatctcaagcacgtggctccgaaggagaagaacaagcataactacgttaaggtgcagaggaggctgcagcgcctcaaccacccggacgtgccgaacaccatctggcctaagtccaacatcggcttcaccatcaggcgcaagaacggcaagtacatcctcaacgtggtgcataaactgccgaagaacaaggtgaagaagtccgtgaagccgaagttcggcgacatcctcatcggcgttgaccagaaccagaccactaaccacacttgtagcatctacaaggtggttaagaagaacaccaaggaggccctgctcgtgccggagtccgacttctacctgaagaagatcgagaccatcaaggtgaccagcttcaccaaggcccgctacaactccgagccgatcgaccagctccattacgagggcatctccgtggacaacgaggtgttcaagaactggtgcaaggaccgcgagcagttcgtggacagcctgtccatcaaggagttcaagaacgagttcaagaggattaaaaacaagaacgagaacctgtactccttcaacgccgactacctgtggctgctgaagcgcatcatctccggcaagctcaacaagaagaagttcgacgtgtcagtgttcgagaagtcaatcaggaacgagatcctcgccatgtgctccaaggagggcctcggcccgctgagggtgtcctccctcagcagcaactccctgaaaagcattggcttcctgaagtccgccatctgctcattcatcagcatcgccctcaaccgcaagggcatcgaggataagaccgatgtgcagaagaacaagatcgaccctgagctgttcgacctgatcggcaagatcgagcagaagcgcgtgaacaagcgcatggagaagaccaggaggaacgctgacttcatcctcaccatggccgttgactaccagaagtccagccagaagaacgtgttcctcttctgcgagggcaacctggagaccgccaagaccggcaactcaaagaagcgcaacagcgccaacgttgattggtgctcacgcaagctgttcgacttcctgaaggagaagagcctcaggcacggcatctacttccacgccgtgaccccgcactacaccagccaccaggacccgttcgagtaccacccgtccaacaaggtgatgctcccgaggttcgccaagttcgataagaacaacccgatccaggattgggctgagaaaaaatatctcggcttcgctaactccgacccggagagcggcaccgccctgtactataaaaaaggcgtggagaacttcttcgctcactaccagaagggcttcaaggagaaggtggagctggccgagatgaaaaacgtgctcaactccaacctgaagaacggcaacctcgagcacgttttctgtccgatccgcggcggccgctactacctgagcacccatccggtgaccagcgacgccaagccgttcgagttcaacggcaggaagtgctacatctgcgacagcgacgaggtggccgccaccaacatcatgctgatcggcctcttctacgtg

SEQ ID NO: 21 - DNA, 3237 bp, synthetic construct
  source 1..3237: mol_type = other DNA
atgcctaagcagaaggacctgccctattcctgcctgtcctacctccagcccaacgagagaaagttgaaactgctcaacaatacttatgaccacctcacccatgggtccaagataatgttcgacaccctgatcgccctgatgggtgggatcaaccccaagatggacgtcatctccgagaacaaggattccgaaataaagaacaacagggacccccaaaccatgtgcgccaccatctggttcaggcccatgaagtccaaaagaattaataaggtctggtccccaaaacagctgaaagagcagttccttaagtactaccaggagtacgaggccgacgtgaagatcaacgatatggttgaagcctactttgactcccccctcggggagaattacgtctgggtggactgcaggaagaaatacaaacagctcgttaaggagctcgcctccatcgccaagaccaccgaggctaaccttaaggaggacctggactgcgaccttgagtgcctgttcaggccctccgagaagaagatgaaactctacggatccaacaaatcttgggcaatcatctccaacctgttcggcgagggggacaaggaggacaggtccaagaagatcaaaatcctgacaaaggcaatccagatccttaccgaatcaaaccccgagtcctacgctgacgtgcagaaggcctttctcgccgccgcagacatcgacgaccccaagaagttccacacacaggagatatggggaaacggctcccccggcaatatcgtgaagatggccagaggcgatttcctcggcaaagagtttgactgtgagaagatcctggaaaagatcaacgatgttctgaaagagaagaccttggattttgacctgaaggttaggctgtctttcaaagagtacctgatttccaagattggtcactattaccagaacagttggtccgagatgatcaactccgctttcgccgacatcatcagtaaaaataccaggaacgtcaacttcgccaaggagaaggttcagctgcagaagaccctgagtgagacttccaacgccaaggtggagctgctgaccgacttttttaagtctgacttcttcttgggcgacgacaagttcgacatcgcccctcataacctggggggggccaatgggatcaagttcttctacgacttctgcaagaagaatgaagaccaatactttttggaggaactcttgctcgaggcagccattgaggagtccgtggccgaagctaaatcaaaatccctcaaggagccacacaaggatctcttgaggtatgtcttctccattaggaaagagaccaccttcgaggagcttagagacgccgcaaagtacattcagacccacaagaggataaagaatatgtctgtccaccccaccgttaagtccgacatcgggttcaatgttacctccggctccgccctggtggggcacgtggttagtccctccaaaaaaattaacgggaggatcgccggagaatcaggattcatctggatctgcatgaaactttgggaggggggagacaagtggatcgagcatcacatcccattcaccgacactagattctatgagcagatctacaaatataaccctgactccaaacttgagcccgtggttctgaggaccaagaggtacggggtggacctcaccaagttcaacctcccccccatgaagacagaccttaagcacgtcgcccccaaggaaaagaacaaacacaactacgtgaaggtgcagagaaggctccagaggctcaaccaccccgatgttcccaacaccatatggcccaagtccaacatcggtttcaccatcaggagaaagaatgggaagtatatcctcaacgtcgttcataaactccccaagaataaggtcaaaaagtccgtcaagcctaagttcggtgatatactgattggggtggaccagaaccagaccactaaccacacctgctccatctacaaggtcgtcaagaagaacaccaaggaggcactcctggtgcctgaatccgacttctacctcaagaagattgagaccatcaaggtcacttccttcaccaaagccaggtacaactcagaacccatcgatcaactccactatgaaggcatctccgtcgacaacgaggtcttcaagaattggtgtaaggacagggagcagtttgttgattccctgtcaatcaaggagtttaagaacgagttcaagagaattaaaaacaagaatgaaaatctgtactccttcaacgctgactacctctggctcctcaaaaggattatatccgggaagctcaacaagaaaaagtttgacgtgtccgtgttcgagaagtccataaggaacgagatcctcgccatgtgctccaaggaggggctcggccccctcagagtgtcttccctctcctccaactccttgaaaagcattggcttcctcaagtccgctatctgcagttttatctccatcgccctcaataggaagggcatcgaagacaagaccgacgtccagaagaacaaaatcgatcccgagctgttcgacctcatcgggaagatcgagcaaaaaagggtgaataagagaatggagaaaaccaggaggaacgccgatttcatattgacaatggccgtggattaccagaagtcctcccaaaagaacgtctttctgttctgcgagggcaacttggagacagccaaaaccggcaactccaaaaagaggaattcagccaacgttgattggtgctccaggaagcttttcgacttcttgaaggaaaagtccctgaggcacggaatctatttccatgccgtgaccccccactacacatcccaccaagatccattcgagtaccacccatccaataaggtcatgctccccaggttcgccaagttcgacaaaaacaaccccatccaggactgggccgagaaaaaatatcttggctttgccaactccgaccccgaaagtggcaccgccctgtattataaaaaaggtgtcgaaaacttcttcgcccattaccaaaaagggttcaaggagaaggtcgagctggccgaaatgaaaaacgtgttgaactctaacctgaagaacggcaacctggagcatgtgttctgtcccatcaggggaggcaggtattacttgtccacccaccccgttacttccgatgccaaaccctttgaatttaacggcaggaagtgctacatctgtgactccgacgaggttgccgccaccaacatcatgctgatcggtcttttctacgtc

SEQ ID NO: 22 - DNA, 3237 bp, synthetic construct
  source 1..3237: mol_type = other DNA
atgccaaagcaaaaagacctgccctactcctgcttgtcctacctgcagcccaacgagaggaagttgaagctgctgaacaacacctacgaccaccttacccacggctctaaaatcatgtttgacacactcatcgcccttatgggggggatcaaccccaaaatggatgttatatccgagaacaaggactccgagatcaaaaataatagggacccccaaaccatgtgcgcaaccatctggtttaggcccatgaagtccaagaggatcaacaaggtctggtcccccaagcagctcaaggagcagttcctgaagtactaccaagagtacgaggccgatgtcaagattaacgatatggtcgaggcctactttgactcccccctcggtgagaactacgtctgggtggactgcaggaagaagtacaagcagctcgttaaggagctggcctctatagctaagaccaccgaggctaatctgaaggaggacctcgactgcgacttggaatgcctgttcaggccctccgagaagaagatgaagctttatgggtccaacaagtcctgggccatcatctccaacctcttcggggaaggtgataaggaggacaggagtaagaagatcaaaatcctcaccaaagccatccagatacttaccgagtccaaccctgagtcatacgccgacgtgcagaaggctttcctggccgccgcagacatcgacgaccccaaaaaattccacacccaggaaatctgggggaacgggtcccccggaaacatcgttaagatggccaggggggacttcctggggaaggaattcgactgcgagaagatcctggagaagattaatgacgttctcaaagagaagaccctggacttcgacctcaaagtgaggttgtcattcaaggagtacctgatctccaagataggtcactattatcagaactcctggtcagagatgatcaactccgcctttgccgatatcatctctaaaaacaccaggaacgtgaacttcgccaaggagaaggttcagctgcagaagaccttgtccgagacctccaacgccaaggtcgaactgcttaccgacttctttaagtccgattttttccttggcgacgacaagtttgatatagccccccacaacctcggcggggccaacgggataaagttcttctacgacttctgcaagaagaatgaggaccagtatttcctggaagagctcctgctcgaagccgccatcgaggagtcagtggccgaggctaaatccaagtccctgaaggaaccccacaaggatctcctgaggtacgtgttctccatcaggaaagaaactacatttgaggaactcagggatgccgccaaatacatccagactcacaaaagaatcaagaatatgtccgtccaccccacagttaagtccgacattggattcaacgtcaccagtggttccgctcttgtgggacacgtcgtgtccccctccaaaaaaattaacgggaggatcgccggggagtctggcttcatatggatctgcatgaaactctgggaaggcggcgacaagtggattgaacaccacattcccttcaccgacactagattctacgagcagatctacaaatataaccccgactccaagttggagccagtggtgctcaggaccaagaggtacggagtcgacttgaccaagtttaacctcccccccatgaagactgacctcaaacacgttgcccccaaggagaaaaacaagcacaattatgtgaaggtccagaggaggctgcagaggctcaatcatcccgatgtgcctaatactatctggcctaagagtaacatcggcttcaccatcaggagaaaaaacggcaagtatatcctcaacgtcgttcataaactgccaaagaacaaagttaagaaatccgttaaacccaaattcggagacatcttgatcggcgtggaccagaatcagaccactaaccacacatgttccatttataaagttgttaagaagaacacaaaggaggccctcctggtgccagagtccgacttctacctcaagaagatcgagaccatcaaagtcacctcctttaccaaggccaggtacaactccgagcccatcgaccaactgcactacgaggggatctccgtggacaacgaggttttcaagaactggtgcaaggatagggagcagtttgtcgattccttgtccatcaaggaattcaagaatgagtttaagaggattaaaaacaagaatgaaaacctgtattccttcaatgccgactacctctggctgctgaaaagaatcatctccggcaagcttaacaagaaaaagttcgacgtctccgtgtttgaaaagtccatcaggaacgaaatcctggccatgtgcagtaaggagggcttggggcccctcagagtctctagtctctcctccaactcactcaaaagcattgggttcttgaagtccgcaatctgctccttcatttccattgccctcaacaggaagggcatcgaggacaaaaccgatgtgcagaagaacaagatagaccccgagctcttcgatctgattgggaagatcgaacaaaagagagtgaacaagaggatggagaaaaccagaagaaacgccgactttatactcacaatggccgtggactaccagaagtcctcccaaaagaatgttttcctcttctgtgagggcaacctcgagaccgccaaaaccggaaactccaagaagagaaattccgctaatgtcgattggtgcagtaggaagctgttcgacttcctgaaagaaaagtcccttaggcatgggatctacttccatgccgtgaccccccactacacctcccatcaagaccctttcgagtaccatccctccaacaaggttatgcttcccaggttcgcaaagttcgacaagaacaacccaatccaggattgggcagaaaaaaaatatctcggtttcgccaactccgaccccgagtccggtaccgccctgtattataaaaaaggagtcgagaacttctttgcacattatcagaaggggttcaaggagaaggtcgagctcgccgagatgaaaaatgtcctgaactccaacctgaagaacggcaacctcgagcacgtcttctgtcctatcaggggcggcaggtactacttgtcaacccaccccgtgacctctgacgccaagcccttcgagttcaacggcagaaagtgctacatctgtgactccgacgaggtggccgccaccaatatcatgcttatcggcctgttctacgtc

SEQ ID NO: 23 - DNA, 3237 bp, synthetic construct
  source 1..3237: mol_type = other DNA
atgcccaagcaaaaggacctgccctactcctgtctgagttacttgcagcctaacgagaggaaactcaagctgttgaacaacacctatgaccatctgacccacgggtccaagatcatgtttgacaccctgatcgccctcatgggaggtatcaacccaaagatggatgtcatcagtgagaacaaggactccgagatcaagaacaacagggatccccagactatgtgcgccaccatttggtttagacctatgaagtccaagaggataaataaagtctggtcccccaagcagcttaaggaacagttccttaaatattaccaagaatacgaagctgacgttaaaatcaacgacatggttgaggcctatttcgactcccccctcggggagaactacgtctgggtggactgcaggaagaaatacaagcagctggttaaggagttggcctcaatcgccaagactaccgaggccaacctgaaggaggatctggactgcgatctcgagtgcctgtttaggccttccgagaaaaagatgaagttgtatgggtccaataagagttgggcaataatatccaacctctttggggagggcgacaaggaggacaggtccaagaagatcaagattctgaccaaagctatccagatccttacagaatccaatcccgagtcctatgccgacgtccagaaggccttcctcgctgccgccgacatcgatgatccaaagaagttccacacccaggagatctggggcaatggctcccccggcaacatagttaaaatggccaggggcgatttccttggtaaagagttcgactgcgagaagatcctcgagaagatcaatgacgttctcaaggagaaaacactcgacttcgatctcaaagtgagactctccttcaaagaataccttatctcaaagatcggccactactaccagaactcctggtccgaaatgatcaattccgccttcgctgacatcatctccaagaataccaggaacgtcaacttcgccaaggagaaagttcaactgcagaagaccctctctgagacttccaatgccaaggtggagctgctgaccgacttctttaagtctgacttcttcctcggcgacgacaagtttgacatcgccccacacaaccttggcggcgccaacgggatcaaattcttttacgatttctgtaagaaaaacgaggatcaatacttcctcgaagagctgctcttggaagcagccatcgaggagtccgtcgccgaggcaaagtccaagtccttgaaagaaccccacaaagacctgcttaggtacgtgttctccatcaggaaggagaccacctttgaggagctgagggacgccgcaaagtatatccaaacccacaagaggatcaagaacatgtcagtccaccccaccgtcaagtctgacatcggtttcaacgtgacctccgggtccgccctcgtgggacatgtcgtgtccccctccaagaaaattaacggaaggatcgccggcgaatccggattcatctggatttgcatgaaattgtgggaggggggtgacaaatggattgaacaccacattcctttcaccgacaccagattctacgagcagatttacaaatataaccccgattccaagctcgaacccgtggtgctgaggaccaagaggtacggggttgacctcaccaaatttaacctccccccaatgaagactgacctgaagcatgtggcccccaaggaaaagaacaagcacaactacgtgaaggtgcagagaagactgcagaggctgaaccaccccgacgttcccaacaccatctggcccaaaagtaatatcgggttcacaatcaggagaaagaacggaaagtatatcttgaatgtcgtccataaattgcctaagaataaggttaagaagtccgtcaaacccaagttcggtgatatcttgatcggggtggatcagaaccagaccaccaatcacacctgctccatctacaaggttgtcaagaagaacacaaaagaggccctcctcgtgcccgagtccgacttttacctgaagaagatcgaaactatcaaggtcacctccttcaccaaggccagatacaactccgagcccatcgaccagctgcattacgagggtatctccgtggacaacgaagtgttcaaaaattggtgcaaggacagggaacagttcgtcgactccctctccatcaaagagttcaagaacgagttcaagaggattaaaaacaagaacgagaacctctactccttcaacgccgactatctgtggctccttaagaggataatctccggcaagctcaataagaagaaatttgacgtgagtgtgttcgagaagtccataaggaacgagatactcgctatgtgctccaaggagggcctggggccccttagggtctcatccctctcctccaactccctcaaaagcattggcttcctgaaatcagccatctgctcctttatttctatcgcactgaacaggaagggcatagaggacaaaactgatgttcagaagaataagatcgaccccgagctcttcgacctgattgggaagatcgaacagaagagggttaacaagaggatggaaaaaaccagaaggaatgccgacttcattctcaccatggccgtcgactaccaaaaatcttcccaaaagaacgtctttctgttctgcgaagggaatcttgagacagccaagaccgggaactccaagaagaggaactccgccaacgtcgactggtgttccagaaaactgttcgacttcctgaaggagaagtccctcaggcacggcatctacttccacgccgtcaccccccactacacatcccaccaggatccctttgagtaccatccatccaacaaggtgatgctgcccaggtttgctaagttcgacaagaacaaccccatccaggactgggccgagaaaaaatatctcggattcgccaactccgaccccgagagtgggaccgccctctactataaaaaaggggtcgagaacttcttcgcacattaccagaaggggttcaaggagaaggtggagttggccgagatgaaaaatgttctgaactctaacctgaagaacggcaacctcgagcacgttttctgccccatcagaggaggcaggtattacctgtccacccaccccgttacctccgacgccaagcccttcgagttcaacgggaggaaatgttacatctgcgactcagatgaggtggccgccaccaacatcatgcttataggcttgttctatgtc

SEQ ID NO: 24 - DNA, 3237 bp, synthetic construct
  source 1..3237: mol_type = other DNA
atgcctaaacagaaggaccttccctactcctgcctctcttatctccagcccaacgagaggaagctcaagttgctgaacaacacctacgaccacttgactcacgggtctaagatcatgttcgacacactgatcgcactcatggggggaatcaaccccaagatggatgtgatttccgagaacaaggattccgagataaaaaacaatagggacccccaaaccatgtgcgccactatctggttcaggcccatgaagtccaagagaatcaacaaggtgtggtcccccaagcagcttaaggagcagttcctcaagtactatcaggagtatgaagcagacgtcaaaatcaacgacatggttgaagcatacttcgattccccccttggcgagaattacgtctgggtcgattgcaggaagaagtataagcagctcgtcaaagagctcgcctccatagctaagaccaccgaggccaaccttaaggaagatctggactgcgatctcgagtgcctgttcaggccctccgagaagaagatgaagctgtacggctccaacaagtcatgggccatcatttcaaacttgttcggcgagggcgacaaggaggacaggtccaagaagatcaagatactcaccaaagccatccagatcctcaccgagtccaaccccgagtcctacgccgacgttcagaaagccttcttggccgccgccgacattgacgatcccaaaaagttccacacccaggagatctgggggaacggttcccccggcaacatcgtcaagatggccaggggcgacttcctcggtaaggagttcgactgtgagaagatcctcgagaaaattaacgatgtcttgaaggagaaaaccctcgacttcgacctcaaagtgaggctcagtttcaaggagtatttgatttccaagatcggccactactatcaaaactcctggtccgagatgatcaactccgccttcgccgatattatctccaagaacaccagaaacgtgaactttgctaaggagaaagtccaactccaaaagaccctttccgagacctccaacgccaaggttgagcttctcactgactttttcaaatccgactttttcttgggggacgataagttcgacatcgcaccccataacctgggcggcgcaaacgggatcaagtttttctacgacttctgcaagaagaatgaggatcagtactttctcgaagaacttctgcttgaggccgccattgaagagtccgtcgctgaggccaagtccaaatccctcaaagagccccacaaagacctgctgaggtacgttttttccatcagaaaggagaccaccttcgaggagctgagggatgctgccaagtacatccagacccacaaaaggatcaagaacatgtccgttcaccccaccgtcaagagtgacatcgggttcaacgtcacaagtgggtccgccctggtgggacacgttgtgtccccctcaaagaaaattaacgggaggatcgccggcgagagtggcttcatatggatatgcatgaaactgtgggagggcggggacaagtggatcgagcaccacatcccttttaccgacaccaggttctacgagcagatctacaaatataaccccgactccaagctggaacccgttgttctcaggaccaaaaggtacggcgtggacctgaccaaattcaacctgccacccatgaagaccgacctgaagcacgtggcccccaaggagaagaacaagcataactacgtcaaggttcagagaaggcttcagagactgaatcatccagacgttcccaatacaatctggcctaaatcaaatataggcttcaccatcaggaggaagaacgggaagtatatcctcaacgtggtccataaactgcctaagaacaaagtcaagaagtccgttaagcccaagttcggggacatattgatcggagttgaccagaatcagaccactaaccatacctgctccatctacaaggtcgtgaagaaaaataccaaggaggccctgctggtgcccgagtcagacttctacctgaaaaagatagaaacaatcaaggtcacctccttcaccaaggctaggtacaactccgaacctatcgaccaactgcattatgagggaatctccgtggacaacgaagtcttcaaaaattggtgcaaggatagggagcaattcgtcgactccctctctatcaaggagtttaagaacgagttcaagaggattaaaaacaaaaacgaaaacctctattcttttaatgccgactacctctggctcctcaagagaatcatctccggtaagctgaacaagaaaaagttcgacgtgtctgtgttcgaaaagtccatcaggaatgagatcctcgccatgtgctccaaggagggcctgggtcccctgagggtctccagtctctcctccaactcccttaaaagcattggtttcctcaagtccgccatttgctccttcatatccatcgctctcaataggaaagggatcgaagacaaaaccgacgtgcagaagaacaagatcgacccagagctgttcgacctgatcgggaagatcgagcagaaaagggtcaacaagagaatggaaaagactaggaggaatgcagacttcatcctgacaatggccgtggattaccagaagtcctcccagaagaatgttttcctcttctgcgaagggaacctcgaaaccgccaaaaccggaaattccaagaagagaaactccgccaacgtcgactggtgttccagaaagctgttcgactttctgaaggagaagtccctgaggcacgggatctactttcacgccgtcactccccactacacctcacaccaggatccattcgagtaccacccctccaacaaggtcatgcttcccagatttgccaaatttgataagaacaaccccatccaggactgggccgagaaaaaatatctgggcttcgccaattccgaccccgagagtgggacagccctctactataaaaaaggcgttgaaaacttctttgcccactaccaaaagggcttcaaggaaaaggtggagttggccgaaatgaaaaatgtgcttaactccaacctcaagaacggtaatttggagcacgtgttctgtcccatcaggggtgggaggtactatctgtccacccatcccgtgacctccgacgccaaaccattcgagtttaacgggaggaagtgttacatctgcgactctgacgaggtggccgccaccaacatcatgttgattggactgttctacgtt

SEQ ID NO: 25 - DNA, 3237 bp, synthetic construct
  source 1..3237: mol_type = other DNA
atgcccaagcagaaggatctcccatactcctgtctgtcctatctgcaacccaacgagaggaagctcaagctgctgaacaacacctacgaccatctcacccacgggtccaagatcatgttcgacaccctgatcgccctcatgggcggcataaaccccaagatggacgtgatctccgaaaacaaggactccgaaatcaaaaacaacagggatccccagaccatgtgcgccaccatctggttcagacccatgaagtccaagaggatcaacaaggtttggtcccccaagcagctcaaggaacagttcttgaaatactaccaagagtacgaggctgacgtcaaaatcaacgatatggtggaggcctactttgactcccccctcggcgagaattacgtctgggtcgactgcaggaagaagtacaagcagttggtcaaggagctggcatctatcgccaagaccacagaggccaacctcaaggaagacctggattgtgatcttgagtgcctcttcaggcccagtgagaagaagatgaagttgtacggatccaataagtcctgggcaatcatctcaaacctcttcggcgagggtgacaaggaggacaggtcaaagaagataaagatccttacaaaagccatccagatcctgaccgagagtaatcccgagtcttatgccgacgttcagaaggcctttctcgcagccgccgacatcgacgacccaaagaaattccacactcaggaaatctgggggaacggatcccccgggaatattgtgaagatggctaggggggactttctcggcaaggaattcgactgcgaaaagatccttgagaagatcaacgacgtcctcaaggagaagaccctcgacttcgatctgaaggttaggctcagtttcaaagagtatcttatctccaagattggacactactaccagaattcctggtccgagatgatcaattccgccttcgccgacatcatctccaaaaacaccaggaacgtcaacttcgccaaggaaaaagtgcaacttcagaagaccctctccgagacatcaaacgccaaggtggagctcctgaccgactttttcaaatcagatttcttccttggcgatgataaattcgacatagccccccataaccttgggggggccaacggcatcaagttcttttacgatttctgcaaaaagaacgaggaccagtactttctggaggaactcctgttggaggccgccatcgaagagtccgttgccgaggctaagtccaagtccctcaaggaaccccacaaagatctcttgaggtacgtcttctccatcagaaaagagactaccttcgaagaactcagggacgccgccaagtatatccagactcacaaaaggatcaagaacatgtctgtgcatcctaccgtgaagtccgacatcgggttcaacgttacctccggttccgcactggtcggccacgttgtttccccctccaagaaaattaacggcaggatagccggagagtccgggttcatatggatatgcatgaagctgtgggagggaggggataagtggattgagcaccacatccccttcaccgacaccaggttctacgaacagatctacaaatataaccccgacagtaagctggaacccgtggtcctcagaaccaagaggtatggcgtggacctgaccaagttcaatttgccccccatgaaaactgacctgaagcacgttgcccctaaggagaagaacaagcacaactatgtgaaggtgcagaggaggctgcaaaggcttaatcatcctgacgtccctaataccatctggcccaagtccaacattggcttcaccatcagaaggaaaaacggtaagtatatcctcaacgtggtccataaacttccaaaaaacaaggtgaagaagtccgttaaacccaaatttggggacatcctcatcggagtcgaccagaatcaaaccaccaaccacacatgctccatatataaagtcgtgaagaaaaacaccaaggaggccttgctcgtgccagaatccgacttttatctgaaaaagatcgagaccatcaaggtcacttcattcaccaaagccaggtacaattcagagcccattgaccaacttcactacgaggggattagtgttgacaacgaggtcttcaagaactggtgcaaagacagggaacagttcgttgactccctctccatcaaggagttcaagaacgagttcaagaggattaaaaataagaacgagaacttgtactcctttaacgccgactatttgtggctgctcaaaaggatcatttccgggaaactgaacaagaagaagttcgacgtctccgtttttgagaagtccataaggaacgagattctggccatgtgctccaaggagggtctcggccctctgagagtgtcctccctctcttccaactctctgaaaagcattggtttcctgaagtccgccatctgctccttcatctccatcgccctcaacagaaaggggatcgaggataagaccgacgtccagaagaacaaaatcgaccccgagctgtttgacctgatcgggaagatcgaacagaagagagtgaataagaggatggagaagaccaggagaaacgcagacttcatcctgacaatggctgttgactaccaaaagtcctcccagaagaacgtgttcctgttctgtgaaggcaacctcgagaccgccaagaccgggaactccaagaaaaggaactccgccaacgttgattggtgctccagaaagcttttcgacttcttgaaagagaagagtttgaggcacggcatttacttccatgctgtcactccccactacacctcccaccaggacccctttgagtaccacccctccaacaaggtcatgctccccaggttcgccaagtttgacaaaaataatcccatacaggactgggccgagaaaaaatatctcggattcgccaactccgacccagagtccggcaccgccctgtactataaaaaaggcgtggagaactttttcgcccactaccagaaggggttcaaagagaaagtcgagctggccgagatgaaaaacgtcctgaattctaacctcaagaatgggaaccttgagcatgtgttctgtcccatcaggggtggcagatactacctctccacccaccccgtgacctccgatgctaagccctttgagttcaacgggaggaagtgctacatctgcgactccgatgaggttgcagccacaaatatcatgctgatcggcctgttctacgtg

SEQ ID NO: 26 - DNA, 3237 bp, synthetic construct
  source 1..3237: mol_type = other DNA
atgcccaagcagaaggacctcccctactcctgcctgtcctacctccagcccaatgagaggaagctgaagctcttgaacaacacctatgaccacttgacccatggctccaagataatgtttgacacccttatcgccctcatggggggcataaaccccaagatggacgtgatctccgagaacaaggactccgagatcaagaacaacagagatccccagaccatgtgtgccaccatctggtttaggcctatgaagtctaagaggatcaacaaggtttggtcccccaagcagcttaaggaacagtttctcaaatattaccaggagtacgaggccgacgtcaagatcaatgacatggtcgaggcctactttgattcccccctgggcgagaactatgtgtgggtggactgtaggaagaagtacaagcaactcgtgaaggagctcgcatccattgccaagacaactgaggcaaacctcaaggaagacctcgattgtgatctcgagtgcctgttcaggccctccgagaagaagatgaagctttacgggtccaacaagtcctgggcaatcatcagtaacctgtttggtgagggagacaaggaagacagatcaaagaaaatcaagattctgaccaaggcaatccagatcctgaccgagtccaaccccgaatcctacgccgacgtccagaaagccttcttggccgccgctgacattgatgaccccaaaaagtttcatactcaagagatctgggggaatgggagtcccgggaacatcgtgaaaatggctaggggcgacttccttggaaaggaattcgactgcgagaaaatcctcgagaagatcaacgacgtgctgaaagaaaagaccctcgacttcgacctgaaggtgaggctgagtttcaaggagtaccttatctccaagatcggccactattaccagaattcctggtccgagatgatcaactccgccttcgccgacatcatctccaagaatacaaggaacgtgaacttcgccaaggagaaagttcaattgcaaaagacactctccgagacatccaacgccaaggttgaactgctgaccgacttcttcaagtccgacttcttcctcggtgacgacaagttcgacatcgcccctcacaatttggggggggccaacggcataaagttcttctacgacttttgcaaaaaaaacgaggaccagtactttctggaggagctcctcctggaggccgctatcgaagaatctgtggctgaggccaagtctaagtccttgaaggagccccataaggacctcctcaggtacgttttcagtatcaggaaggagactactttcgaagagctgagagacgccgccaagtacatccagacccacaagaggataaaaaacatgtccgtccatcccaccgtcaagtccgacatcggcttcaacgtcacctccggcagtgccctcgtcgggcacgtcgtttccccctccaaaaaaattaacgggagaatcgccggtgagtccgggttcatatggatctgcatgaagctctgggaggggggggataaatggatcgagcaccacattccctttaccgataccagattctacgaacagatctacaaatataaccccgactccaagttggagcccgttgttctgaggaccaagaggtacggtgttgacctgaccaagttcaacctgccacccatgaaaaccgacctcaagcacgtggcccccaaggaaaagaacaagcataactatgtgaaagttcagaggaggctgcagaggctgaaccaccctgacgtgcccaataccatctggcccaagtccaacatcggcttcacaataaggaggaagaatggaaagtacatcctcaacgtggtgcataaactcccaaaaaacaaggtgaagaagtccgtcaagcctaagttcggcgatatcctgatcggcgtggaccaaaaccagaccaccaatcatacctgctccatctacaaagtcgttaagaagaacaccaaggaggccctcctcgtgcccgaatccgatttctaccttaagaagatagagaccattaaagttacctccttcaccaaggccaggtataactccgagcctattgatcagctgcactacgaggggatctccgtcgataacgaggttttcaaaaactggtgcaaggatagggagcagttcgtcgattctctgtccatcaaagagttcaaaaatgagttcaagaggattaaaaacaagaatgaaaatctctactccttcaacgccgattacttgtggctgctgaagagaatcatctctggtaagcttaataagaagaagtttgacgtgtccgtcttcgagaagtcaatcagaaacgagatcctcgccatgtgctccaaggagggcctgggcccactgagggtgtcctccttgtcatccaactcattgaaaagcattggcttcctcaagtccgccatctgctccttcatctccattgcattgaatagaaaaggaatagaggataagactgacgtccagaaaaataagatcgaccccgagttgttcgacctgattgggaaaattgaacaaaaaagggtgaacaagagaatggagaaaaccaggaggaacgctgactttatcctgaccatggccgtggactaccagaaatcatcccagaaaaatgtttttcttttctgcgaggggaaccttgagaccgccaagaccggtaactccaagaagagaaactccgccaatgttgactggtgctccaggaagctcttcgactttctgaaggagaagtccctcaggcacggtatctatttccacgccgttaccccacactacacctctcaccaggaccccttcgagtatcacccctccaacaaggtcatgctgcccaggtttgccaagttcgacaaaaacaaccccatccaagactgggccgaaaaaaaatatctcggatttgccaattccgatccagaatccgggacagccctgtactataaaaaaggcgtcgagaacttcttcgcacactaccagaagggcttcaaggagaaagtcgagctggccgagatgaaaaacgtcctcaactccaaccttaaaaacgggaacctcgaacacgtcttctgtcccatcaggggcggaaggtattacctttccacccaccccgtcaccagtgacgccaagcccttcgaattcaacggcaggaaatgctatatctgcgactcagacgaggtcgctgccactaacataatgctgatcgggctcttctacgtg

SEQ ID NO: 27 - DNA, 3237 bp, synthetic construct
  source 1..3237: mol_type = other DNA
atgcccaagcagaaggaccttccctactcctgtctgtcctacctgcagcccaacgaaagaaaactcaagctcctcaacaacacctacgaccaccttactcacgggagtaaaatcatgttcgataccctgatcgccctcatgggcggcatcaaccccaagatggatgtgatttccgaaaataaagactcagaaatcaagaataacagggacccccagaccatgtgcgccaccatatggttcaggcccatgaagtccaagaggatcaacaaggtgtggtcacccaagcagcttaaggagcagttcctcaagtactaccaagagtacgaggccgacgttaagataaacgacatggtcgaggcctacttcgactccccccttggcgagaactacgtgtgggttgactgcaggaagaaatacaagcagttggtcaaagaactggcttccattgccaagaccaccgaggctaacctgaaggaggacctggattgtgacctggagtgcctgtttaggccctccgagaagaaaatgaaactgtacgggtccaacaaatcctgggccatcatctccaatctcttcggcgagggtgacaaggaggacaggtccaagaaaattaaaatcctcacaaaggccatccaaatcctcaccgagtccaaccccgagtcctatgccgacgtccagaaggccttcttggccgccgccgatatcgacgatcccaaaaagttccacactcaggagatctggggaaatggttctcccggaaacatcgtcaagatggccagaggcgacttcctgggcaaggagtttgactgtgaaaagatcctggagaagattaacgacgtgctgaaggagaagaccctcgactttgacctcaaggttagactctccttcaaggagtacctgatttccaagatcgggcactactatcagaactcctggagtgagatgatcaactccgcctttgccgatatcatctccaagaacaccaggaacgtcaatttcgccaaggagaaggtgcagctgcagaagaccctgtccgagacctccaacgcaaaagtcgagctcttgaccgacttcttcaaatcagatttcttcctgggcgacgataagttcgacatcgctccacataacctggggggggccaacgggatcaaatttttctatgacttctgcaagaagaacgaagaccagtatttcctcgaggagctcctcctggaagccgccatcgaggagtccgtcgccgaggctaagtctaagtccttgaaggagccccacaaggacctcttgaggtacgtcttctccattaggaaggagaccacatttgaggagctgagggacgccgccaaatacatacaaactcataagagaatcaaaaatatgtccgtgcaccctaccgtcaagtctgacatcgggtttaacgtgacatccggttcagctctcgtcgggcacgtggtgtccccttccaaaaaaattaacggaaggatcgccggggaatcagggttcatctggatctgcatgaaactttgggagggaggcgacaagtggatcgagcaccatatccccttcaccgataccaggttctacgagcagatctacaaatataaccccgacagtaagttggaacccgttgtcctgaggaccaagaggtacggcgttgacctcaccaagtttaaccttcctccaatgaagaccgacttgaagcacgtggcccccaaggaaaagaacaagcacaactacgtcaaggtgcagaggaggctccagaggctcaaccaccctgacgttcccaacaccatctggcccaagtctaacatcgggttcaccatcagaaggaagaatggaaagtatatcctgaacgtcgtccataaactgcccaagaataaggtgaagaaatccgtcaagccaaagttcggtgatatacttataggcgtggatcaaaaccagaccactaaccacacctgctccatctataaggttgtcaagaagaacaccaaagaggccctcctcgtccccgagtccgacttttacctgaaaaaaattgagactattaaggtgacttccttcaccaaagccaggtacaactccgagcccatcgaccagctgcactatgaaggcatctccgttgacaacgaggtttttaagaactggtgcaaggacagagagcaattcgtggactccctgtccatcaaggagttcaaaaacgaattcaagaggattaaaaacaagaatgagaacctctattccttcaatgccgactacctctggctgctgaaaagaatcatctccggcaagctgaacaagaagaagttcgacgtttcagtcttcgaaaagtccatcagaaacgagatccttgccatgtgctccaaggagggcctcgggcccttgagggtgtcctccctctccagtaactccctgaaaagcattggttttctcaagagtgccatctgttccttcatctccatcgcactgaataggaaagggatagaggacaagaccgacgtgcagaagaacaagatcgatcccgagctgttcgatctcataggaaagatagagcaaaagagagtgaacaagaggatggaaaagacaagaaggaatgccgatttcatcttgaccatggccgtggactaccagaaatccagtcagaagaatgttttcttgttttgcgagggcaacctcgagaccgcaaagacaggcaactccaaaaagaggaattccgccaatgtcgactggtgctctaggaaactctttgacttcctgaaggaaaagtccctcaggcatggaatatacttccatgcagtcacaccccactacacctcccaccaggatccattcgagtaccacccctccaataaggttatgcttcccaggttcgccaaattcgacaagaacaaccccatccaggactgggccgaaaaaaaatatcttggttttgccaactccgaccccgaatccgggaccgctctctactataaaaaaggggttgagaacttcttcgcacactatcaaaagggcttcaaggagaaagtggaactggccgagatgaaaaacgtcctcaattccaacttgaagaatgggaaccttgagcatgtgttctgccccattaggggcggcaggtactacctctctacccacccagtcacctccgacgcaaaaccttttgaattcaacgggaggaagtgctacatctgcgactccgatgaggttgccgccaccaacatcatgcttatcgggctgttctatgtt

SEQ ID NO: 28 - DNA, 3237 bp, synthetic construct
  source 1..3237: mol_type = other DNA
atgccaaagcagaaagacctcccctattcctgcttgtcctatctccaaccaaacgagaggaagctcaaactgctgaacaacacatatgaccacctgacccatggctccaagattatgtttgacacactcatcgccctgatgggcggtatcaatcccaagatggatgtgatttccgaaaacaaggactccgagatcaagaacaatagagacccccaaaccatgtgcgccacaatctggttcaggcctatgaagtccaagaggatcaataaggtctggtcacctaaacagcttaaggagcaattcctcaagtactaccaggagtacgaggccgacgtcaagatcaacgatatggtcgaggcctacttcgattccccactcggagagaactacgtttgggtcgactgtaggaagaagtataagcagcttgtgaaggaacttgcctccatcgccaagacaactgaggccaacctcaaggaggacttggattgcgatcttgaatgcctcttcaggccctccgagaagaagatgaaactgtacggctccaacaagtcctgggccatcatctccaacctgttcggggagggagacaaggaggacaggtccaagaaaatcaagatcctcaccaaagccatccagatcctgaccgagtccaaccccgagtcctacgccgacgtgcaaaaggccttcttggctgcagccgacatagacgaccccaagaagttccacacccaggagatctgggggaacgggtctcctggtaatatcgtcaagatggcaagaggtgatttcctggggaaggaatttgactgcgagaagatcttggagaaaattaacgatgtgctgaaagaaaagacactggacttcgacctgaaagtgagactgtccttcaaggagtacctgatctccaagatagggcattactaccagaattcttggtccgagatgatcaacagtgccttcgccgacattatctcaaagaacaccaggaacgttaacttcgccaaggaaaaggtgcagctgcagaagaccctgtccgaaaccagtaacgccaaggtggagctcttgaccgacttcttcaagtccgacttcttcctcggcgatgacaagttcgacatcgccccccataacttggggggcgccaacgggatcaagttcttctatgacttttgcaagaagaacgaggaccagtacttcttggaagaactgctgctcgaggccgccatcgaggagtccgtcgccgaggccaaatccaagtcccttaaggaaccccacaaggacctccttaggtacgtcttctccatcaggaaggaaactaccttcgaggaactgagggacgcagccaaatacatccagacccacaagaggatcaaaaacatgtccgttcaccccaccgtgaagtctgatattggattcaacgttacctccggaagtgctctggtgggccacgttgtttccccctccaagaaaattaacgggagaatcgccggggagtccggctttatctggatctgcatgaagctctgggaggggggggataagtggatagagcatcacatcccctttaccgacaccaggttttacgagcaaatctacaaatataaccccgactccaagctggagcccgtcgttctcaggaccaaaaggtatggggttgacctgaccaagtttaacctccccccaatgaagaccgaccttaagcacgttgcacccaaggaaaagaacaagcacaactacgtcaaggttcagaggagactccagaggctcaaccaccccgacgtccccaacactatctggcctaagtccaacataggcttcaccatcaggaggaagaacggcaagtacatcctcaatgtggtgcataaactccccaagaacaaggtgaagaagtccgtcaaacccaagttcggcgacatcctgatcggggtggaccagaaccagaccaccaaccacacctgctccatctacaaggtcgtgaagaagaacaccaaggaggccttgctggtccccgagtccgacttctacctcaaaaagattgaaacaattaaagttacttccttcaccaaagccaggtacaattccgaacccatcgatcagctgcactacgagggcatctcagtcgataatgaggtctttaaaaactggtgtaaggatagggagcagttcgtcgactcactgtcaatcaaggagttcaaaaacgaatttaagagaattaaaaataagaacgagaacctgtattccttcaacgccgactacctctggctcctcaagaggataatctccggcaagctcaataaaaaaaagttcgacgtgtccgtcttcgaaaagtccatcaggaacgagatcctcgcaatgtgctccaaggaggggctggggcccctgagggtgtcctctctctcttccaacagtctgaaaagcattggattcctgaaatccgctatttgctcctttatatccatcgccctcaacaggaagggcatcgaggacaagaccgacgtccagaagaacaaaatcgacccagaattgttcgacctcatcgggaaaatcgagcagaaaagggtgaacaaaaggatggagaaaaccagaaggaacgccgactttatccttaccatggctgtggactaccagaagtcctcccaaaagaatgtttttttgttttgcgaggggaatctggagaccgccaagaccggcaattcaaagaaaagaaattccgccaacgtcgactggtgctccagaaagcttttcgacttccttaaggagaaatccttgagacacggaatatacttccacgccgtgaccccccactataccagtcaccaggacccctttgaataccatcccagtaataaggttatgctccccaggtttgccaagttcgacaagaacaatcccatccaggactgggccgagaaaaaatatctgggcttcgccaattccgatcccgagtctggcaccgccctctactataaaaaaggcgtcgagaacttcttcgcccactaccaaaagggtttcaaggagaaggtcgagctggccgagatgaaaaacgttttgaactccaacctcaaaaacggtaatcttgagcacgttttctgccccattaggggtggcaggtactacctctccacccatcccgtgacttccgacgcaaaacctttcgagttcaacgggaggaaatgttatatatgtgattccgatgaggtcgctgctactaacataatgctcatcggtctgttctatgtg

SEQ ID NO: 29 - DNA, 3162 bp, synthetic construct
  source 1..3162: mol_type = other DNA
atgtcctcagccatcaagtcctacaagtccgtgctgcgcccgaacgagaggaagaaccagctcctgaagtccaccatccagtgcctggaggatggctccgctttcttcttcaagatgctccagggcctcttcggcggcatcaccccggagatcgtgcgcttctccaccgagcaggagaagcagcagcaggacatcgctctctggtgcgccgtgaactggttccgcccggtgagccaggactccctcacccacaccatcgcttccgacaacctcgtggagaagttcgaggagtactacggcggcactgcctccgatgccatcaagcagtacttcagcgccagcatcggcgagtcatactactggaacgactgcaggcagcagtactacgacctctgcagggagctgggcgtggaggtgagcgacctcactcacgacctggagatcctgtgcagggagaagtgcctggccgttgctaccgagagcaaccagaacaacagcatcatctcagtgctcttcggcaccggcgagaaggaggaccgctccgttaagctgcgcatcaccaagaagatcctggaggccatcagcaacctgaaggagatcccgaagaacgtggcccctatccaggagatcatcctcaacgtggccaaggccactaaggagaccttcaggcaggtgtacgccggcaacctgggcgccccgagcactctggagaagttcatcgccaaggacggccagaaggagttcgatctgaagaagctccagaccgacctgaagaaggtgatccgcggcaagagcaaggagcgcgactggtgctgccaggaggagctgaggtcatacgtggagcagaacaccatccagtacgacctgtgggcctggggcgagatgttcaacaaggcccacaccgccctgaaaattaaaagcacccgcaactacaacttcgccaagcagaggctcgagcagttcaaggagatccagtccctcaacaacctgctcgtggtgaagaagctgaacgacttcttcgactcagagttcttctccggcgaggagacctacaccatctgcgtgcatcacctcggcggcaaggacctgtccaagctgtacaaggcctgggaggacgaccctgccgacccggagaacgccatcgtggtgctctgcgacgatctgaagaacaacttcaagaaggagccgatcaggaacatcctgcgctacatcttcaccatccgccaggagtgtagcgcccaggacatcctggccgccgccaaatataaccagcagctcgacaggtataaaagccagaaggccaacccgtccgtgctgggcaaccagggcttcacctggaccaacgccgtgatcctcccggagaaggcccagaggaacgacaggccgaactccctggacctccgcatctggctgtacctgaagctgcgccatccggacggcaggtggaagaagcaccacatcccgttctacgacacccgcttcttccaggagatctacgccgccggcaactccccggtggacacctgccagttccgcaccccgcgcttcggctaccacctcccgaagctcaccgatcagaccgccatcagggtgaacaagaagcacgtgaaggccgctaagaccgaggcccgcatccgcctcgccatccagcagggcaccctcccggtgtcaaacctcaagatcaccgagatcagcgccaccatcaactccaagggccaggttaggatccctgtgaagttcgatgttggccgccagaagggcaccctgcagatcggcgaccgcttctgtggctacgaccagaaccagaccgcctcacacgcctacagcctctgggaggttgtgaaggagggccagtaccacaaggagctcggctgtttcgtgcgcttcatctcctccggcgatatcgtgtccatcactgagaacaggggcaaccagttcgaccagctgtcctacgagggcctggcctacccgcagtacgctgactggaggaagaaggccagcaagttcgtgagcctgtggcagatcaccaagaagaacaagaagaaggagatcgtgaccgttgaggccaaggagaagttcgatgccatctgcaagtaccagccgaggctgtacaagttcaacaaggagtacgcctacctcctgcgcgacatcgttaggggcaagtcactcgtggagctccagcagatcaggcaggagatcttcaggttcatcgagcaggactgcggcgtgacccgcctgggctcactcagcctctccactctcgagaccgtgaaggctgtgaagggcatcatctacagctacttcagcaccgccctgaacgcttcaaagaacaaccctatcagcgacgagcagaggaaggagttcgacccggagctgttcgccctcctggagaagctcgagctgatccgcacccgcaagaagaagcagaaggtggagcgcatcgctaactccctcatccagacctgcctggagaacaacatcaagttcatccgcggcgagggcgacctcagcaccaccaacaacgccaccaagaagaaggccaacagccgctccatggactggctcgcccgcggcgtgttcaacaagatcaggcagctggccccgatgcacaacatcactctgttcggctgcggctccctgtacacttcccaccaggacccgctcgtgcacaggaaccctgacaaggccatgaaatgcaggtgggccgccatcccggtgaaggacatcggcgactgggtgctgcgcaagctcagccagaacctccgcgccaagaacatcggcaccggcgagtactaccatcagggcgtgaaggagttcctgagccactacgagctccaggacctcgaggaggagctcctgaagtggaggtccgacaggaagtccaacatcccgtgctgggtgctccagaacaggctcgccgagaagctcggcaacaaggaggctgtggtttacatcccggtgaggggcggcaggatctacttcgccacccataaagtggctaccggcgccgtgagcatcgtgttcgaccagaagcaggtgtgggtttgcaacgccgaccacgtggccgccgccaacatcgccctcaccgtgaagggcatcggcgagcagagcagcgacgaggagaacccggacggcagcaggattaaactgcagctcaccagc

SEQ ID NO: 30 - DNA, 3162 bp, synthetic construct
  source 1..3162: mol_type = other DNA
atgagcagcgccatcaagtcctacaagtccgtgctgaggccgaacgagcgcaagaaccagctgctcaagagcaccatccagtgtctggaggacggcagcgccttcttcttcaagatgctgcagggcctgttcggcggcatcaccccggagatcgtgcgcttctcaactgagcaggagaagcagcagcaggacatcgccctgtggtgcgctgtgaactggttcaggccggtgagccaggactcactcactcacaccatcgcctccgacaacctcgttgagaagttcgaggagtactacggcggcaccgcctccgacgccatcaagcagtacttcagcgccagcatcggcgagtcatactactggaacgattgcaggcagcagtactacgatctgtgcagggagctcggcgtggaggtgagcgacctgacccacgacctggagatcctgtgcagggagaagtgcctggctgtggccaccgagagcaaccagaacaacagcatcatcagcgtgctgttcggcactggcgagaaggaggaccgctcagtgaagctccgcatcaccaagaagatcctcgaggccatctccaacctcaaggagatcccgaagaacgtggccccgatccaggagatcatcctcaacgtggccaaggctaccaaggagactttccgccaggtgtacgccggcaacctgggcgcccctagcaccctcgagaagttcatcgccaaggatggccagaaggagttcgacctcaagaagctgcagaccgacctgaagaaggttatcaggggcaagtccaaggagcgcgactggtgctgccaggaggagctcaggtcctacgtggagcagaacaccatccagtacgatctgtgggcctggggcgagatgttcaacaaggcccacaccgccctcaaaattaaaagcacccgcaactacaacttcgccaagcagcgcctcgagcagttcaaggagatccagagcctgaacaacctgctggtggtgaagaagctcaacgacttcttcgactccgagttcttctccggcgaggagacctacaccatctgcgtgcaccacctcggcggcaaggacctcagcaagctctacaaggcctgggaggacgacccggccgacccggagaacgccatcgtggtgctgtgcgacgacctcaagaacaacttcaagaaggagcctatccgcaacatcctcaggtacatcttcaccatccgccaggagtgttccgcccaggacatcctggccgccgccaaatataaccagcagctcgacaggtataaaagccagaaggctaacccttccgtgctgggcaaccagggcttcacctggaccaacgccgttatcctcccggagaaggcccagaggaacgacaggccgaactcactggacctccgcatctggctgtacctgaagctgcgccacccggacggccgctggaagaagcaccacatcccgttctacgacacccgcttcttccaggagatctacgccgccggcaactcaccggtggacacctgccagttcaggacccctaggttcggctaccacctgccgaagctgaccgaccagactgccatcagggttaacaagaagcatgttaaggccgccaagaccgaggcccgcatccgcctcgccatccagcagggcactctcccggtgagcaacctcaagatcaccgagatctccgccaccatcaactccaagggccaggtgcgcatccctgtgaagttcgacgttggcaggcagaagggcaccctccagatcggcgacaggttctgcggctacgaccagaaccagaccgcctcacacgcctactcactctgggaggtggtgaaggagggccagtaccataaggagctcggctgtttcgtgcgcttcatctcctccggcgacatcgtgtccatcaccgagaacaggggcaaccagttcgaccagctgtcctacgagggcctcgcctacccgcagtacgccgattggaggaagaaggcttccaagttcgttagcctctggcagatcaccaagaagaacaagaagaaggagatcgtgaccgtggaggccaaggagaagttcgatgccatctgcaagtaccagccgaggctctacaagttcaacaaggagtacgcctacctgctcagggacatcgtgaggggcaagagcctcgtggagctgcagcagatcaggcaggagatcttcaggttcatcgagcaggactgcggcgtgaccaggctcggcagcctcagcctgagcactctcgagaccgttaaggctgtgaagggcatcatctactcctacttcagcaccgctctgaacgccagcaagaacaacccgatctcagatgagcagcgcaaggagttcgacccggagctcttcgccctcctcgagaagctggagctgatcaggaccaggaagaagaagcagaaggtggagcgcatcgctaacagcctcatccagacctgcctcgagaacaacatcaagttcatcaggggcgagggcgacctcagcaccaccaacaacgccaccaagaagaaggccaactcccgctccatggactggctcgccaggggcgtgttcaacaagatccgccagctcgccccgatgcacaacatcaccctcttcggctgcggcagcctgtacacctcccaccaggacccgctggtgcacaggaacccggacaaggccatgaaatgccgctgggccgctatcccggtgaaggacatcggcgattgggtgctgaggaagctcagccagaacctgcgcgccaagaacatcggcaccggcgagtactaccaccagggcgtgaaggagttcctgtcccattacgagctgcaggacctcgaggaggagctgctgaagtggcgctccgaccgcaagtccaacatcccgtgctgggtgctgcagaaccgcctcgccgagaagctgggcaacaaggaggccgtggtgtacatcccggtgcgcggcggccgcatctacttcgccacccataaagtggctaccggcgccgtgtcaatcgtgttcgaccagaagcaggtgtgggtgtgcaacgctgaccacgttgccgccgccaacatcgccctgactgtgaagggcatcggcgagcagtccagcgacgaggagaacccggacggctcccgcattaaactgcagctgacctcc

SEQ ID NO: 31 - DNA, 3162 bp, synthetic construct
  source 1..3162: mol_type = other DNA
atgagctccgccatcaagagctacaagtcagtgctcaggccgaacgagaggaagaaccagctgctgaagagcaccatccagtgcctggaggacggctcagccttcttcttcaagatgctgcagggcctcttcggcggcatcaccccggagatcgttcgcttctccaccgagcaggagaagcagcagcaggacatcgccctgtggtgtgccgtgaactggttcaggccggtgagccaggactccctgacccacaccatcgcctccgacaacctggtggagaagttcgaggagtactacggcggcaccgctagcgatgccatcaagcagtacttcagcgcctccatcggcgagtcctactactggaacgactgcaggcagcagtactacgacctgtgtagggagctcggcgtggaggtgtccgacctgacccacgacctggagatcctctgcagggagaagtgcctcgccgtggccaccgagtccaaccagaacaactccatcatctcagtgctcttcggcaccggcgagaaggaggaccgctccgtgaagctgaggatcaccaagaagatcctcgaggccatcagcaacctgaaggagatcccgaagaacgtggccccgatccaggagatcatcctcaacgtggctaaggccaccaaggagaccttcaggcaggtgtacgctggcaacctcggcgccccgtcaaccctggagaagttcatcgccaaggacggccagaaggagttcgacctcaagaagctccagactgacctcaagaaggttatcaggggcaagtccaaggagcgcgattggtgctgccaggaggagctgaggagctacgtggagcagaacaccatccagtacgacctgtgggcttggggcgagatgttcaacaaggcccacaccgccctcaaaattaaaagcaccaggaactacaacttcgctaagcagcgcctggagcagttcaaggagatccagtccctcaacaacctcctcgttgtgaagaagctcaacgacttcttcgactccgagttcttctccggcgaggagacctacaccatctgcgtgcaccacctcggcggcaaggatctgagcaagctgtacaaggcttgggaggacgacccggccgatccggagaacgccatcgtggtgctgtgcgatgacctgaagaacaacttcaagaaggagccgatccgcaacatcctgcgctacatcttcactatccgccaggagtgctccgcccaggacatcctcgccgccgccaaatataaccagcagctcgaccgctataaaagccagaaggccaacccgagcgttctgggcaaccagggcttcacttggaccaacgccgttatcctcccggagaaggcccagaggaacgaccgcccgaactcactggatctccgcatctggctgtacctcaagctcaggcatcctgacggccgctggaagaagcaccacatcccgttctacgacacccgcttcttccaggagatctacgccgccggcaactcaccggtggacacctgccagttccgcaccccgcgcttcggctaccatctgccgaagctcaccgaccagaccgccatccgcgtgaacaagaagcacgttaaggccgccaagaccgaggcccgcatcaggctggccatccagcagggcaccctgccggtgtccaacctgaagatcaccgagatcagcgctactatcaacagcaagggccaggtgcgcatcccggtgaagttcgatgtgggccgccagaagggcaccctccagatcggcgacaggttctgcggctacgaccagaaccagaccgcttcacacgcctacagcctgtgggaggtggtgaaggagggccagtaccacaaggagctgggctgcttcgtgaggttcatctcaagcggcgacatcgtgagcatcaccgagaacaggggcaaccagttcgaccagctctcctacgagggcctcgcctacccgcagtacgccgactggaggaagaaggcctccaagttcgtgtccctgtggcagatcaccaagaagaacaagaagaaggagatcgtgactgtggaggccaaggagaagttcgacgccatctgcaagtaccagccgaggctgtacaagttcaacaaggagtacgcctacctcctcagggacatcgtgaggggcaagagcctggtggagctgcagcagatcaggcaggagatcttccgcttcatcgagcaggactgcggcgtgaccaggctcggctccctcagcctgtccaccctggagaccgtgaaggccgtgaagggcatcatctactcatacttcagcaccgccctcaacgcctccaagaacaacccgatcagcgacgagcagaggaaggagttcgacccggagctcttcgccctcctcgagaagctggagctgatccgcactcgcaagaagaagcagaaggtggagcgcatcgccaacagcctcatccagacctgcctggagaacaacatcaagttcatccgcggcgagggcgacctcagcaccaccaacaacgccaccaagaagaaggccaacagcaggtccatggactggctcgcccgcggcgtgttcaacaagatccgccagctggcccctatgcacaacatcactctcttcggctgtggctccctctacacctcccaccaggacccgctggtgcacaggaaccctgacaaggccatgaaatgccgctgggccgccatcccggtgaaggacatcggcgactgggttctcaggaagctgagccagaacctcagggccaagaacatcggcaccggcgagtactaccaccagggcgttaaggagttcctcagccactacgagctgcaggacctcgaggaggagctcctgaagtggcgctccgaccgcaagagcaacatcccttgctgggttctccagaacaggctggctgagaagctgggcaacaaggaggctgtggtgtacatccctgtgcgcggcggccgcatctacttcgccacccataaagtggccaccggcgctgtgtcaatcgtgttcgatcagaagcaggtgtgggtgtgcaacgccgaccatgtggccgccgccaacatcgccctgactgtgaagggcatcggcgagcagtcctccgacgaggagaacccggacggcagcaggattaaactccagctgaccagc

SEQ ID NO: 32 - DNA, 3162 bp, synthetic construct
  source 1..3162: mol_type = other DNA
atgagctcagctatcaagagctacaagagcgtgctgaggccgaacgagcgcaagaaccagctgctcaagtccaccatccagtgtctggaggacggcagcgccttcttcttcaagatgctccagggcctcttcggcggcatcaccccggagatcgtgcgcttcagcaccgagcaggagaagcagcagcaggacatcgccctctggtgcgccgtgaactggttcaggccggttagccaggacagcctcactcacactatcgccagcgacaacctcgtggagaagttcgaggagtactacggcggcaccgccagcgatgccatcaagcagtacttctccgcctccatcggcgagtcatactactggaacgactgccgccagcagtactacgacctctgccgcgagctcggcgtggaggtgtccgatctgacccacgacctggagatcctctgccgcgagaagtgcctggccgtggctaccgagagcaaccagaacaactccatcatcagcgttctgttcggcactggcgagaaggaggaccgctcagtgaagctcaggatcaccaagaagatcctggaggccatctcaaacctgaaggagatccctaagaacgtggctccgatccaggagatcatcctcaacgttgccaaggccaccaaggagactttcaggcaggtgtacgccggcaacctgggcgccccttccaccctcgagaagttcatcgccaaggacggccagaaggagttcgacctgaagaagctccagactgacctcaagaaggtgatcaggggcaagagcaaggagcgcgactggtgctgccaggaggagctccgctcctacgttgagcagaacaccatccagtacgatctctgggcttggggcgagatgttcaacaaggcccacaccgccctcaaaattaaatccaccaggaactacaacttcgccaagcagcgcctcgagcagttcaaggagatccagagcctgaacaacctgctcgtggtgaagaagctgaacgacttcttcgactccgagttcttcagcggcgaggagacctacaccatctgcgtgcaccatctgggcggcaaggacctcagcaagctctacaaggcctgggaggatgacccggccgacccggagaacgccatcgttgtgctgtgcgacgacctcaagaacaacttcaagaaggagccgatcaggaacatcctcaggtacatcttcaccatcaggcaggagtgtagcgcccaggacatcctggccgccgccaaatataaccagcagctggaccgctataaaagccagaaggccaacccgagcgtgctcggcaaccagggcttcacctggaccaacgccgtgatcctgccggagaaggcccagcgcaacgacaggcctaacagcctcgacctgaggatctggctctacctcaagctgcgccacccggacggcaggtggaagaagcaccacatcccgttctacgacaccaggttcttccaggagatctacgccgccggcaactccccggtggacacttgtcagttccgcaccccgcgcttcggctaccacctgccgaagctcaccgaccagaccgccatccgcgtgaacaagaagcatgttaaggccgccaagaccgaggccaggatcaggctggccatccagcagggcaccctcccggtgtccaacctcaagatcactgagatctccgccaccatcaactccaagggccaggtgcgcatcccggtgaagttcgacgtgggccgccagaagggcaccctccagatcggcgatcgcttctgcggctacgaccagaaccagaccgcttcccacgcctacagcctctgggaggtggtgaaggagggccagtaccataaggagctcggctgcttcgtgaggttcatctccagcggcgacatcgtgagcatcaccgagaaccgcggcaaccagttcgaccagctgagctacgagggcctggcctacccgcagtacgccgattggcgcaagaaggccagcaagttcgtgtccctgtggcagatcaccaagaagaacaagaagaaggagatcgttaccgtggaggctaaggagaagttcgacgccatctgcaagtaccagccgaggctgtacaagttcaacaaggagtacgcctacctgctccgcgacatcgtgaggggcaagtccctcgtggagctgcagcagatcaggcaggagatcttccgcttcatcgagcaggactgcggcgtgaccaggctgggctcactgtccctgagcaccctggagaccgttaaggccgtgaagggcatcatctactcatacttcagcaccgccctcaacgcctccaagaacaacccgatctccgacgagcagaggaaggagttcgacccggagctgttcgccctgctcgagaagctggagctgatccgcaccaggaagaagaagcagaaggtggagcgcatcgctaacagcctcatccagacctgcctggagaacaacatcaagttcatccgcggcgagggcgacctgtccaccaccaacaacgctaccaagaagaaggccaactcccgctcaatggactggctcgcccgcggcgtgttcaacaagatcaggcagctcgccccgatgcacaacatcaccctcttcggctgcggctcactctacacctcccaccaggacccgctggtgcaccgcaaccctgacaaggccatgaaatgccgctgggctgccatcccggtgaaggatatcggcgattgggtgctgaggaagctctcacagaacctgcgcgccaagaacatcggcaccggcgagtactaccaccagggcgtgaaggagttcctctcccactacgagctgcaggacctcgaggaggagctcctgaagtggaggtccgacaggaagagcaacatcccgtgctgggtgctccagaacaggctcgccgagaagctgggcaacaaggaggccgtggtgtacatcccggtgaggggcggcaggatctacttcgccacccataaagtggccaccggcgccgtgtcaatcgttttcgaccagaagcaggtgtgggtgtgcaacgccgaccacgtggccgccgctaacatcgctctgactgtgaagggcatcggcgagcagtcctccgacgaggagaaccctgacggcagcaggattaaactgcagctgacctcc

SEQ ID NO: 33 - DNA, 3162 bp, synthetic construct
  source 1..3162: mol_type = other DNA
atgtccagcgccatcaagagctacaagtcagtgctccgcccgaacgagaggaagaaccagctcctcaagagcaccatccagtgcctcgaggacggctccgccttcttcttcaagatgctgcagggcctcttcggcggcatcaccccggagatcgtgaggttctccaccgagcaggagaagcagcagcaggacatcgccctctggtgcgccgtgaactggttcaggccggtgtcccaggactccctgactcacaccatcgcctccgataacctggtggagaagttcgaggagtactacggcggcaccgcctccgatgccatcaagcagtacttctcagccagcatcggcgagagctactactggaacgactgcaggcagcagtactacgacctgtgcagggagctcggcgttgaggtgtccgacctcacccacgacctcgagatcctctgtcgcgagaagtgcctggccgtggctaccgagtccaaccagaacaacagcatcatcagcgttctgttcggcaccggcgagaaggaggacaggagcgtgaagctgaggatcaccaagaagatcctggaggctatctccaacctcaaggagatcccgaagaacgtggccccgatccaggagatcatcctgaacgttgccaaggctaccaaggagaccttccgccaggtttacgccggcaacctgggcgccccgagcaccctcgagaagttcatcgccaaggacggccagaaggagttcgacctgaagaagctccagaccgacctcaagaaggtgatcaggggcaagtccaaggagcgcgactggtgctgtcaggaggagctgaggagctacgtggagcagaacactatccagtacgacctgtgggcctggggcgagatgttcaacaaggcccataccgctctgaaaattaaaagcacccgcaactacaacttcgccaagcagcgcctcgagcagttcaaggagatccagtcactcaacaacctgctggtggtgaagaagctcaacgacttcttcgacagcgagttcttcagcggcgaggagacctacaccatctgcgttcaccacctcggcggcaaggacctgtccaagctgtacaaggcctgggaggacgacccggccgatccggagaacgccatcgtggtgctctgcgacgacctcaagaacaacttcaagaaggagccgatcaggaacatcctgaggtacatcttcaccatccgccaggagtgctccgcccaggacatcctcgccgccgccaaatataaccagcagctggacaggtataaaagccagaaggccaacccgtccgtgctgggcaaccagggcttcacttggaccaacgccgtgatcctgccggagaaggcccagcgcaacgacaggcctaactcactggacctccgcatctggctctacctcaagctccgccacccggacggccgctggaagaagcatcacatccctttctacgacacccgcttcttccaggagatctacgctgctggcaactccccggtggacacctgccagttcaggaccccgaggttcggctaccacctcccgaagctgactgaccagaccgccatcagggttaacaagaagcacgtgaaggccgccaagaccgaggcccgcatccgcctggccatccagcagggcaccctcccggtgtccaacctgaagatcactgagatctcagccaccatcaactccaagggccaggtgcgcatcccggttaagttcgacgtgggcaggcagaagggcaccctccagatcggcgaccgcttctgcggctacgaccagaaccagactgcctcccacgcctacagcctgtgggaggtggtgaaggagggccagtaccacaaggagctcggctgcttcgtgaggttcatctccagcggcgatatcgtgagcatcaccgagaacaggggcaaccagttcgatcagctctcctacgagggcctcgcctacccgcagtacgccgactggcgcaagaaggcctccaagttcgtgagcctgtggcagatcaccaagaagaacaagaagaaggagatcgtgaccgtggaggctaaggagaagttcgacgccatctgcaagtaccagcctaggctgtacaagttcaacaaggagtacgcctacctgctccgcgacatcgtgaggggcaagagcctggtggagctgcagcagatcaggcaggagatcttccgcttcatcgagcaggactgcggcgtgacccgcctcggctccctcagcctctcaaccctggagaccgtgaaggccgttaagggcatcatctactcctacttcagcaccgccctcaacgcctcaaagaacaacccgatctcagacgagcagaggaaggagttcgatccggagctgttcgccctcctcgagaagctcgagctgatccgcaccaggaagaagaagcagaaggtggagcgcatcgccaactccctgatccagacttgcctcgagaacaacatcaagttcatcaggggcgagggcgacctcagcaccaccaacaacgccaccaagaagaaggccaacagccgcagcatggactggctcgcccgcggcgtgttcaacaagatcaggcagctcgccccgatgcataacatcaccctgttcggctgcggctccctgtacactagccaccaggacccgctggtgcaccgcaaccctgataaggccatgaaatgtaggtgggctgccatccctgtgaaggacatcggcgactgggtgctgcgcaagctgagccagaacctgcgcgccaagaacatcggcaccggcgagtactaccaccagggcgtgaaggagttcctgagccactacgagctgcaggacctggaggaggagctcctgaagtggaggtccgacaggaagtccaacatcccgtgctgggtgctccagaacaggctcgccgagaagctcggcaacaaggaggctgtggtgtacatcccggtgcgcggcggccgcatctacttcgccacccataaagtggccaccggcgccgtgagcatcgtgttcgaccagaagcaggtgtgggtgtgcaacgctgaccacgtggctgccgccaacatcgccctgaccgttaagggcatcggcgagcagtcatcagatgaggagaacccggacggcagccgcattaaactgcagctcacctcc

SEQ ID NO: 34 - DNA, 3162 bp, synthetic construct
  source 1..3162: mol_type = other DNA
atgtcatccgccatcaagagctacaagagcgtgctgcgcccgaacgagaggaagaaccagctcctgaagtcaaccatccagtgcctcgaggacggcagcgccttcttcttcaagatgctgcagggcctgttcggcggcatcaccccggagatcgtgcgcttctcaactgagcaggagaagcagcagcaggacatcgctctgtggtgtgccgtgaactggttccgcccggtgagccaggactccctcactcacaccatcgctagcgacaacctcgtggagaagttcgaggagtactacggcggcaccgcttcagacgctatcaagcagtacttctccgccagcatcggcgagtcctactactggaacgactgcaggcagcagtactacgacctctgcagggagctgggcgtggaggtgtcagatctgacccatgacctggagatcctctgccgcgagaagtgcctcgccgtggccaccgagtccaaccagaacaactcaatcatctcagtgctcttcggcaccggcgagaaggaggaccgcagcgtgaagctgcgcatcaccaagaagatcctggaggccatcagcaacctgaaggagatcccgaagaacgtggccccgatccaggagatcatcctcaacgtggccaaggccaccaaggagaccttccgccaggtgtacgccggcaacctcggcgccccttccaccctcgagaagttcatcgccaaggacggccagaaggagttcgacctgaagaagctgcagaccgacctcaagaaggttatcaggggcaagagcaaggagagggactggtgctgccaggaggagctgaggtcctacgttgagcagaacaccatccagtacgacctgtgggcctggggcgagatgttcaacaaggcccacaccgccctcaaaattaaatccacccgcaactacaacttcgccaagcagaggctggagcagttcaaggagatccagtccctgaacaacctcctggtggtgaagaagctgaacgacttcttcgattccgagttcttctcaggcgaggagacttacaccatctgcgtgcatcacctgggcggcaaggacctgagcaagctgtacaaggcctgggaggacgatccggccgacccggagaacgccatcgtggttctctgcgacgacctgaagaacaacttcaagaaggagccgatccgcaacatcctgaggtacatcttcaccatccgccaggagtgcagcgcccaggacatcctggccgccgccaaatataaccagcagctggacaggtataaaagccagaaggccaaccctagcgtgctgggcaaccagggcttcacctggaccaacgccgtgatcctgccggagaaggcccagcgcaacgacaggccgaactccctcgatctccgcatctggctctacctcaagctgaggcacccggacggccgctggaagaagcaccacatcccgttctacgacactcgcttcttccaggagatctacgccgctggcaactccccggtggacacctgtcagttcaggaccccgaggttcggctaccacctcccgaagctgaccgaccagaccgccatcagggtgaacaagaagcacgttaaggccgccaagaccgaggcccgcatccgcctcgccatccagcagggcaccctgccggtgtccaacctcaagatcaccgagatcagcgccaccatcaactccaagggccaggtgcgcatcccggtgaagttcgacgtgggcaggcagaagggcactctgcagatcggcgacaggttctgcggctacgaccagaaccagaccgccagccacgcctacagcctgtgggaggtggtgaaggagggccagtaccacaaggagctgggctgcttcgtgaggttcatctcctccggcgatatcgtgtccatcactgagaaccgcggcaaccagttcgaccagctcagctacgagggcctcgcctaccctcagtacgccgattggcgcaagaaggcttccaagttcgtgagcctctggcagatcaccaagaagaacaagaagaaggagatcgtgaccgttgaggccaaggagaagttcgacgccatctgcaagtaccagccgcgcctctacaagttcaacaaggagtacgcctacctgctcagggacatcgtgcgcggcaagtcactggtggagctgcagcagatcaggcaggagatcttccgcttcatcgagcaggactgcggcgtgactcgcctcggctccctctccctctcaaccctggagaccgttaaggccgtgaagggcatcatctacagctacttcagcaccgccctgaacgcttccaagaacaacccgatcagcgacgagcagcgcaaggagttcgacccggagctgttcgccctcctcgagaagctcgagctcatcaggacccgcaagaagaagcagaaggtggagaggatcgccaacagcctcatccagacctgcctcgagaacaacatcaagttcatcaggggcgagggcgacctcagcaccaccaacaacgccaccaagaagaaggccaactccaggtccatggactggctggcccgcggcgtgttcaacaagatccgccagctggctccgatgcacaacatcaccctcttcggctgcggctccctctacaccagccaccaggacccgctcgtgcaccgcaaccctgacaaggctatgaaatgccgctgggccgccatcccggtgaaggacatcggcgactgggttctgaggaagctgagccagaacctcagggccaagaacatcggcaccggcgagtactaccaccagggcgtgaaggagttcctgagccactacgagctccaggatctcgaggaggagctcctcaagtggaggtccgacaggaagagcaacatcccgtgttgggtgctccagaacaggctggccgagaagctgggcaacaaggaggccgtggtgtacatccctgtgaggggcggcaggatctacttcgccacccataaagttgccaccggcgccgtttccatcgtgttcgaccagaagcaggtgtgggtgtgcaacgctgaccatgtggccgccgccaacatcgctctcactgtgaagggcatcggcgagcagagctccgacgaggagaacccggatggctcccgcattaaactccagctgaccagc

SEQ ID NO: 35 - DNA, 3162 bp, synthetic construct
  source 1..3162: mol_type = other DNA
atgtcaagcgctatcaagagctacaagagcgtgctcaggccgaacgagaggaagaaccagctcctcaagagcaccatccagtgcctcgaggacggctccgccttcttcttcaagatgctccagggcctcttcggcggcatcaccccggagatcgtgcgcttctcaaccgagcaggagaagcagcagcaggacatcgccctctggtgcgctgttaactggttccgcccggtgtcccaggattcactgacccacaccatcgcctccgataacctcgtggagaagttcgaggagtactacggcggcaccgccagcgacgccatcaagcagtacttctccgccagcatcggcgagagctactactggaacgactgcaggcagcagtactacgacctgtgccgcgagctcggcgtggaggtgtccgatctcacccatgacctcgagatcctgtgtagggagaagtgcctggccgtggccaccgagagcaaccagaacaactccatcatcagcgttctcttcggcaccggcgagaaggaggaccgcagcgtgaagctgcgcatcaccaagaagatcctggaggctatcagcaacctgaaggagatcccgaagaacgtggctccgatccaggagatcatcctgaacgtggccaaggccaccaaggagaccttcaggcaggtgtacgccggcaacctcggcgccccgtccaccctcgagaagttcatcgccaaggacggccagaaggagttcgacctcaagaagctgcagaccgatctcaagaaggtgatcaggggcaagtcaaaggagagggactggtgctgccaggaggagctgaggtcctacgtggagcagaacactatccagtacgatctctgggcctggggcgagatgttcaacaaggcccacactgccctgaaaattaaaagcactcgcaactacaacttcgccaagcagcgcctggagcagttcaaggagatccagtccctgaacaacctgctcgtggttaagaagctgaacgacttcttcgactccgagttcttctcaggcgaggagacctacaccatctgcgtgcatcacctcggcggcaaggacctgagcaagctctacaaggcctgggaggacgacccggctgatccggagaacgccatcgttgtgctctgcgacgacctgaagaacaacttcaagaaggagccgatccgcaacatcctccgctacatcttcaccatccgccaggagtgttccgcccaggacatcctggccgccgccaaatataaccagcagctggacaggtataaaagccagaaggccaacccgtccgtgctgggcaaccagggcttcacctggaccaacgccgtgatcctgcctgagaaggctcagaggaacgacaggcctaactccctggacctccgcatctggctctacctcaagctcaggcatcctgacggcaggtggaagaagcaccacatcccgttctacgacaccaggttcttccaggagatctacgccgccggcaactcaccggtggacacctgccagttcaggactccgcgcttcggctaccacctgccgaagctcaccgaccagaccgccatccgcgtgaacaagaagcacgtgaaggccgccaagaccgaggctaggatccgcctggccatccagcagggcaccctcccggtttcaaacctgaagatcaccgagatctcagccactatcaacagcaagggccaggtgcgcatcccggtgaagttcgacgtgggccgccagaagggcactctgcagatcggcgaccgcttctgcggctacgaccagaaccagaccgccagccacgcctactcactctgggaggtggtgaaggagggccagtaccacaaggagctcggctgcttcgtgaggttcatctcaagcggcgacatcgtgtccatcaccgagaaccgcggcaaccagttcgaccagctgtcctacgagggcctcgcctacccgcagtacgccgactggcgcaagaaggccagcaagttcgtgagcctctggcagatcaccaagaagaacaagaagaaggagatcgtgaccgtggaggccaaggagaagttcgacgccatctgcaagtaccagccgaggctctacaagttcaacaaggagtacgcctacctgctccgcgacatcgtgcgcggcaagtccctcgttgagctccagcagatcaggcaggagatcttcaggttcatcgagcaggactgcggcgtgacccgcctcggctccctgtccctgtccaccctggagactgtgaaggccgtgaagggcatcatctactcctacttcagcaccgccctcaacgctagcaagaacaacccgatctccgacgagcagcgcaaggagttcgaccctgagctcttcgctctgctcgagaagctggagctcatcaggacccgcaagaagaagcagaaggttgagaggatcgccaacagcctgatccagacttgcctggagaacaacatcaagttcatcaggggcgagggcgacctcagcaccaccaacaacgccaccaagaagaaggccaactcccgctccatggattggctggcccgcggcgtgttcaacaagatccgccagctggccccgatgcacaacatcaccctgttcggctgcggcagcctctacaccagccaccaggacccgctggtgcaccgcaacccggacaaggccatgaaatgcaggtgggccgccatcccggtgaaggacatcggcgactgggtgctgaggaagctgtcccagaacctcagggccaagaacatcggcaccggcgagtactaccaccagggcgtgaaggagttcctgagccactacgagctccaggacctcgaggaggagctgctcaagtggaggtccgacaggaagtccaacatcccttgctgggtgctccagaaccgcctggctgagaagctgggcaacaaggaggccgtggtgtacatcccggtgaggggcggcaggatctacttcgccacccataaagtggccaccggcgccgtgagcatcgtgttcgaccagaagcaggtttgggtgtgtaacgccgaccacgttgctgccgccaacatcgccctgaccgtgaagggcatcggcgagcagagcagcgacgaggagaacccggatggctcccgcattaaactgcagctgacctcc

SEQ ID NO: 36 - DNA, 3162 bp, synthetic construct
  source 1..3162: mol_type = other DNA
atgagcagcgccatcaagagctacaagagcgtgctccgcccgaacgagaggaagaaccagctgctcaagagcaccatccagtgcctggaggacggctcagctttcttcttcaagatgctgcagggcctcttcggcggcatcaccccggagatcgttaggttctccaccgagcaggagaagcagcagcaggacatcgccctctggtgcgccgtgaactggttcaggcctgtgtcacaggactccctcacccacaccatcgcctccgacaacctggtggagaagttcgaggagtactacggcggcaccgccagcgacgccatcaagcagtacttctccgcctccatcggcgagtcctactactggaacgactgcaggcagcagtactacgacctgtgcagggagctcggcgtggaggtgagcgatctgacccacgacctcgagatcctgtgccgcgagaagtgcctcgccgtggccaccgagtccaaccagaacaactccatcatctccgttctgttcggcaccggcgagaaggaggatcgctcagtgaagctccgcatcaccaagaagatcctggaggccatctcaaacctgaaggagatcccgaagaacgtggccccgatccaggagatcatcctgaacgtggccaaggctactaaggagaccttccgccaggtgtacgccggcaacctcggcgccccgtcaactctcgagaagttcatcgccaaggacggccagaaggagttcgacctcaagaagctccagaccgacctcaagaaggtgatccgcggcaagagcaaggagagggactggtgctgccaggaggagctgcgcagctacgttgagcagaacaccatccagtacgacctgtgggcctggggcgagatgttcaacaaggcccataccgccctcaaaattaaaagcacccgcaactacaacttcgccaagcagcgcctggagcagttcaaggagatccagtccctgaacaacctcctcgtggtgaagaagctcaacgacttcttcgattccgagttcttcagcggcgaggagacctacactatctgcgtgcatcacctgggcggcaaggacctgagcaagctgtacaaggcctgggaggacgatccggccgacccggagaacgccatcgtggtgctgtgcgacgatctcaagaacaacttcaagaaggagccgatccgcaacatcctgaggtacatcttcaccatccgccaggagtgtagcgcccaggacatcctggccgccgctaaatataaccagcagctcgaccgctataaaagccagaaggccaacccttccgttctcggcaaccagggcttcacctggactaacgccgtgatcctgccggagaaggcccagaggaacgaccgcccgaacagcctggacctgcgcatctggctgtacctcaagctccgccacccggacggccgctggaagaagcaccacatcccgttctacgacaccaggttcttccaggagatctacgccgctggcaacagcccggtggacacctgccagttcaggaccccgcgcttcggctaccacctcccgaagctgaccgaccagaccgccatccgcgtgaacaagaagcacgtgaaggccgccaagaccgaggctaggatcaggctggccatccagcagggcaccctcccggtgtccaacctgaagatcaccgagatcagcgccactatcaactccaagggccaggtgcgcatcccggtgaagttcgatgtgggccgccagaagggcaccctccagatcggcgacaggttctgcggctacgaccagaaccagaccgcctcacacgcctacagcctctgggaggtggtgaaggagggccagtaccacaaggagctgggctgcttcgttaggttcatctcctccggcgacatcgtgtccatcactgagaacaggggcaaccagttcgaccagctgagctacgagggcctggcctaccctcagtacgctgactggcgcaagaaggcctcaaagttcgtgtccctctggcagatcaccaagaagaacaagaagaaggagatcgtgaccgttgaggccaaggagaagttcgacgccatctgcaagtaccagcctcgcctctacaagttcaacaaggagtacgcctacctcctgagggacatcgtgaggggcaagagcctggtggagctccagcagatcaggcaggagatcttcaggttcatcgagcaggactgcggcgtgaccaggctcggcagcctctcactctcaaccctggagaccgtgaaggccgtgaagggcatcatctactcctacttcagcaccgccctcaacgcctccaagaacaacccgatcagcgacgagcagcgcaaggagttcgacccggagctgttcgccctgctggagaagctggagctgatcaggacccgcaagaagaagcagaaggtggagaggatcgctaactccctcatccagacctgtctcgagaacaacatcaagttcatccgcggcgagggcgacctctccaccactaacaacgccaccaagaagaaggccaactccaggagcatggactggctggcccgcggcgttttcaacaagatcaggcagctcgctccgatgcacaacatcaccctgttcggctgcggcagcctctacacctcccaccaggacccgctcgtgcatcgcaacccggacaaggctatgaaatgtaggtgggccgccatccctgtgaaggacatcggcgactgggtgctcaggaagctgagccagaacctccgcgctaagaacatcggcactggcgagtactaccaccagggcgtgaaggagttcctctcccactacgagctgcaggacctggaggaggagctcctgaagtggcgctccgaccgcaagtccaacatcccgtgctgggtgctgcagaacaggctcgccgagaagctcggcaacaaggaggccgtggtgtacatcccggtgaggggcggcaggatctacttcgccacccataaagttgccaccggcgccgtgagcatcgtgttcgaccagaagcaggtgtgggtgtgcaacgccgaccacgttgctgccgccaacatcgccctcaccgtgaagggcatcggcgagcagtcaagcgatgaggagaacccggatggcagcaggattaaactgcagctgacctcc

SEQ ID NO: 37 - DNA, 3162 bp, synthetic construct
  source 1..3162: mol_type = other DNA
atgtcctccgccatcaaaagttacaagtccgtcctcaggcccaacgagaggaagaatcagctccttaagtccaccattcagtgcttggaggacgggtccgccttttttttcaagatgctgcaggggctcttcggagggataacccccgagatcgtgaggttttccaccgagcaggagaaacaacagcaggacatagccctttggtgtgcagtgaattggtttaggcccgtttcacaggactccctcacccacaccatagccagtgacaacctcgttgagaaattcgaggagtactatggggggaccgcctccgacgccataaagcagtacttctcagcctccatcggcgagtcctactactggaacgattgcaggcagcagtactacgacttgtgtagggaacttggggtggaggtgtccgacctgactcatgacctggaaatcctgtgcagggagaaatgcctcgccgtcgccaccgagtccaaccagaacaactctatcatctccgttttgtttggcaccggggagaaagaggataggtctgtgaagttgaggataaccaaaaagattctcgaggctatctccaaccttaaggagatccccaagaacgttgcacccattcaagagatcatcctcaacgtggccaaagccactaaggaaaccttcaggcaggtgtatgccggaaacttgggggccccttccacactggagaagttcatcgccaaggacggccaaaaggaattcgatctgaaaaagctccagaccgacttgaaaaaggtgatcaggggcaaatccaaagagagagactggtgctgtcaagaggagctgaggtcctacgtcgagcaaaataccatccaatacgatctctgggcctggggcgagatgtttaacaaggcccatacagccctgaaaattaaatcaaccaggaactacaacttcgctaagcagagattggagcagttcaaggagatccagagtctcaacaaccttctggtggtgaagaagctcaacgacttctttgactccgagttcttctcaggggaggagacatacacaatctgcgtgcaccacctcggcggcaaggacctttccaagctttacaaggcctgggaagacgatcccgccgacccagaaaatgctatcgtcgtcctgtgcgatgacctgaaaaataatttcaagaaagagcccatcaggaatatcctcaggtatatcttcactatcaggcaggagtgctccgctcaggatattctggccgccgccaaatataaccagcagctggacaggtataaaagccaaaaggccaacccatccgttcttggtaaccaaggtttcacctggactaacgccgtcatcctgcccgaaaaggcccaaaggaacgacagacccaattccctggacttgaggatctggctgtatctgaagctgagacacccagacggcaggtggaaaaagcaccacattcccttctacgacaccagattcttccaggaaatctacgccgcaggcaactccccagtggatacctgccagttcaggacccccagatttggctaccatctgcccaagctgaccgatcagacagccatcagggtcaataagaagcacgttaaggccgccaagaccgaggcaaggatcagattggccatccaacagggaaccctccccgtctcaaacctcaagatcaccgagatttccgccaccatcaactccaagggccaagtcaggatccccgtgaagttcgacgttggcaggcaaaaggggaccctgcagattggcgacagattttgcgggtatgaccagaaccagaccgcatcccacgcctactccctctgggaggtcgtcaaggagggtcagtatcacaaggaactgggctgcttcgtgaggttcatatcctccggggacatcgtttccatcactgagaacagggggaaccagttcgaccagctgtcctacgaggggctggcttatccccagtacgccgactggaggaagaaggcatccaagttcgtgtccctttggcagatcaccaagaagaacaaaaaaaaggagatcgttaccgtggaagccaaagaaaagttcgacgcaatctgcaaataccaacccaggctctacaagttcaacaaggagtacgcctatcttctgagggacatcgttagaggcaaatccctcgttgagctgcagcagatcaggcaggagatcttcaggtttatcgagcaggactgcggggtcacaaggctcggttccctctcactctctaccctcgaaaccgttaaagcagtcaagggaatcatttacagttacttctccaccgccctgaatgcctccaagaataaccctatctccgatgaacagaggaaggagttcgatcctgagctgttcgccctgctcgagaagctggagctgataaggaccagaaaaaaaaagcagaaagtggaaaggatcgccaactccttgatacagacctgtctcgaaaacaacatcaagttcattaggggcgaaggcgacctctctaccaccaacaatgctactaagaagaaggccaattccagatctatggattggctcgccaggggtgtcttcaacaagatcaggcagcttgcccctatgcacaacatcaccctcttcggttgcgggtccttgtacacctcccatcaagaccccctggtccataggaatcccgataaggccatgaaatgcagatgggctgccatccccgtcaaggatatcggagactgggttctcagaaagctctcccaaaacctcagggccaagaacatcggtacaggagagtactaccaccaaggggtcaaggagtttttgagtcactacgagctgcaggacctcgaggaggagctcctgaagtggaggtccgacaggaagtccaacatcccctgttgggtcctgcagaacagactcgcagaaaagctcggaaacaaggaggccgttgtgtatatccccgtgagagggggcaggatctactttgctactcataaagttgccaccggcgccgtctccatcgtcttcgaccagaagcaggtgtgggtctgcaacgccgaccacgtcgccgccgctaacatcgccctcaccgtgaaggggataggcgaacagtcctccgacgaggagaaccccgacgggtccaggattaaactgcagcttacctcc

SEQ ID NO: 38 - DNA, 3162 bp, synthetic construct
  source 1..3162: mol_type = other DNA
atgtccagtgcaatcaagtcctacaaatcagtgctcagacccaacgagaggaaaaatcagttgctcaagtccaccatccaatgcctcgaggacggctccgcttttttctttaagatgctccagggcctgtttgggggcatcacccctgagatcgtgaggttctcaaccgaacaggagaagcagcagcaagacatcgccctgtggtgcgccgttaattggttcaggcccgtctcccaggattccctgactcacactatcgcatccgacaacctggttgaaaaattcgaggaatattatgggggcacagcctctgatgccataaagcaatacttcagtgcctcaatcggagaatcctactactggaacgactgcaggcagcagtactacgacctgtgcagggagttgggggtcgaggtctccgacctcactcacgatctcgagatcctctgcagggaaaagtgcttggccgtggccaccgagtccaaccagaacaactccatcatctccgtgctcttcggcaccggcgagaaggaggacaggtccgtgaagctgagaatcaccaaaaagatcctggaggccatttccaatctcaaggagatccccaaaaacgtcgctcccattcaagagataatcctgaacgtcgccaaagccaccaaagagaccttcagacaggtctacgccggtaacctcggcgccccatccaccctcgagaaattcatcgccaaagatggccagaaggaattcgatctgaagaagctccagaccgacctcaaaaaggtgatcagggggaaaagtaaggagagggactggtgttgccaggaggagctcaggtcctatgttgagcaaaatacaatccagtacgacctttgggcctggggcgaaatgttcaacaaagcccacaccgcccttaaaattaaatccacaagaaattacaacttcgccaagcagagactggagcagttcaaggagatccaatctctcaacaacctgttggttgtcaagaagctgaacgacttcttcgattccgagttcttctccggtgaggagacctacactatctgtgtccaccaccttgggggcaaggacctgtcaaagctgtacaaagcatgggaggatgaccccgccgatcccgagaacgccatcgtcgtgctctgcgatgacctgaagaacaactttaaaaaagagccaatcaggaatatccttaggtacatctttaccatcaggcaggaatgctccgcccaggacatcctcgccgccgccaaatataaccaacagctcgacaggtataaaagccagaaggccaacccctccgtcctcgggaaccagggattcacctggaccaacgccgtgatcctgccagaaaaggcccagaggaatgataggcccaactccctcgaccttaggatctggttgtacctgaagttgagacaccccgatgggaggtggaagaagcatcacatacccttctacgacacaaggttcttccaggaaatctacgccgccggtaactcccctgtcgacacctgccaatttaggaccccaaggtttgggtaccatctgcccaagctgactgaccagaccgccattagggtcaataagaaacacgtcaaagctgccaagaccgaagctaggatcagactcgcaatccaacagggaacactccccgtgtccaacctcaagatcaccgagatttccgccaccatcaactccaagggacaagtgaggatccccgttaagttcgatgttgggaggcagaagggtaccctccagataggggacagattctgcggctatgaccagaaccaaaccgcatcacacgcttactccctgtgggaagtcgttaaggaagggcagtaccataaggagctgggctgcttcgtcaggttcatatcctccggggacatcgtgtccataaccgagaataggggcaaccagttcgaccagctctcctatgagggacttgcctatccccagtacgccgactggaggaaaaaggcctccaagttcgtctccttgtggcaaataaccaagaagaataagaagaaggagatcgtgaccgtcgaggccaaggagaagttcgacgctatttgtaagtatcagcctaggctgtacaagttcaataaggagtacgcctacctgctgagagacatagtgaggggtaagtccctggtcgaactgcaacagatcagacaggaaatctttaggttcatcgagcaggactgtggggttacaaggctcggttccctctccctgtccacccttgagaccgtcaaggccgtgaagggcatcatctacagttacttcagtaccgccctgaacgcctccaagaacaatcccatctctgacgagcagagaaaggaatttgaccccgagttgttcgctctcctggagaagctcgagctcataaggaccaggaagaagaaacagaaggtggaaaggatcgccaactccctgatccaaacttgccttgaaaacaacatcaagttcatcagaggggagggcgacctgtccaccaccaacaacgccactaagaagaaggccaactccagatccatggactggcttgctaggggcgtttttaacaaaattaggcagctggcccccatgcacaatatcacactttttgggtgcggctccctttacacctcccaccaggaccccttggttcacagaaatcccgacaaagccatgaaatgcaggtgggctgcaattcccgtgaaagacatcggggattgggtcctgaggaagctctctcaaaacctgagagccaagaacatcggcaccggagagtactaccaccagggggtgaaggagttcttgtcccattacgagctccaggatctggaggaggagctcttgaagtggaggtccgacaggaagtccaacatcccttgctgggtgctccaaaacaggcttgccgagaagctgggtaacaaagaggccgtggtgtatatccccgttagggggggcaggatttattttgcaacccataaagttgccaccggagccgtgtccatcgtgttcgaccagaagcaggtctgggtttgtaacgccgaccatgttgcagcagccaacattgccttgaccgttaagggcatcggggagcagtcttccgacgaggagaaccccgacgggtccaggattaaactccagctcacctca

SEQ ID NO: 39 - DNA, 3162 bp, synthetic construct
  source 1..3162: mol_type = other DNA
atgtcctccgccattaagtcctacaagtccgttctgaggcccaacgagagaaagaaccagctgcttaagtccaccatccagtgcctcgaggacggcagtgccttctttttcaagatgttgcagggactgttcggcggaatcacccccgagatcgtgaggttctcaaccgagcaggagaagcaacagcaggacattgccctgtggtgcgccgtcaactggtttaggcccgtttcccaggactcccttacccacaccatcgccagtgacaacctggtggagaagttcgaggagtactacggggggactgcatccgatgcaatcaagcaatacttctccgcatccattggtgagtcctattactggaacgattgcaggcagcaatactacgatttgtgcagagagctgggcgtggaggttagtgatctcactcatgacctggagatcttgtgcagggagaagtgcctcgctgtggccacagaatccaaccagaacaattctatcatctccgtccttttcgggaccggggagaaggaagacaggtccgtcaagctcaggattaccaagaagatcctcgaggccatttcaaatctcaaggagatacccaaaaacgtcgctcccatccaagagatcatcttgaatgtggcaaaagccactaaggagacattcaggcaggtgtatgctggcaacctgggcgccccatcaaccttggaaaagttcatcgccaaggatgggcagaaggaattcgacctcaaaaaattgcaaaccgatcttaagaaggttatcaggggcaaatccaaagaaagggactggtgttgccaggaggaactgaggtcctatgtcgagcaaaacaccatccaatacgatctgtgggcctggggcgagatgtttaacaaggctcatacagccctgaaaattaaatccactaggaactacaactttgcaaagcagaggctggagcaattcaaggagattcagagtttgaacaacctcctcgtcgtgaagaagcttaacgacttcttcgactccgagttcttctcaggcgaggagacttacaccatctgcgtccaccacctgggcggtaaggacctgtccaaactgtacaaggcctgggaggacgaccccgccgacccagagaatgccatagtggtcttgtgtgacgatctgaaaaacaactttaagaaagagcccatcaggaacatactcaggtatatcttcaccatcagacaggagtgctccgctcaagacatcctcgcagccgccaaatataaccagcagctggacagatataaaagccagaaagccaatccatccgttctcggcaaccaggggttcacctggaccaacgccgttatcctccccgagaaagcccagaggaacgacagacccaactccctggacctcaggatctggctgtacctgaaactgaggcaccccgacggaagatggaagaagcatcatatccccttctacgataccaggttcttccaggagatatacgctgccggaaactcccccgtggacacctgtcagttcaggacccccaggttcgggtaccacctgcccaagctcaccgaccagaccgccatcagggttaacaagaagcacgtcaaggccgccaaaacagaggccaggatcaggctcgccatccagcaggggacccttcctgtctccaacctgaagatcaccgagatctccgccaccataaactccaaggggcaagtgaggatcccagtgaagttcgacgttggcaggcaaaaggggacactgcagataggcgacaggttctgcgggtacgaccagaatcagaccgcctctcatgcctactccctttgggaagtggtgaaggaaggtcagtatcacaaggaactcggctgcttcgtgagatttatctcttccggcgacatcgtctccatcaccgagaacagggggaatcaatttgaccagctttcctacgaggggctcgcctacccccagtacgccgactggagaaagaaagcttccaagttcgtctccctctggcaaatcaccaagaaaaacaagaagaaagagatcgtgaccgtcgaagccaaagagaagtttgatgccatttgcaagtaccagcccagactctacaagttcaataaggagtatgcctatcttctgagagatatagttagaggtaagtccctcgtcgagctccagcagatcaggcaggagatattcaggttcatcgagcaggattgcggagtcaccaggttgggctccctctctctgtccacactggagacagttaaggccgtgaagggcatcatctactcctacttctccactgccttgaacgcctctaaaaacaaccccatttccgacgagcagaggaaggagttcgaccctgagttgtttgccctgctggagaagcttgaactcatcaggaccaggaagaaaaagcagaaggttgagagaatcgccaattccctcatccaaacctgcctcgagaataacatcaagtttatcagaggggaaggagacctctccaccaccaacaacgccactaagaaaaaggctaattccaggagtatggactggttggcaaggggggtttttaacaagatcagacagctggcccccatgcacaatattaccctctttgggtgtggctcactctatacctcccaccaggatccccttgtccacagaaaccctgacaaggctatgaaatgcaggtgggccgccatccccgtcaaggacatcggagactgggttctgaggaagctctcccaaaacctcagggccaaaaacatcgggaccggcgagtactaccaccagggcgtcaaggaattcctctcccactatgagctgcaggacctggaagaggagctcctgaagtggaggtccgacaggaagtccaacatcccctgttgggtgctccagaataggctggccgagaagcttggcaacaaagaagccgtcgtgtacatccctgtgaggggtggtaggatctacttcgcaacccataaagtcgccaccggtgccgtctccatcgtgttcgaccagaagcaggtgtgggtgtgcaacgccgaccacgttgcagccgccaacatagccctcaccgttaaagggatcggggaacaaagttccgatgaagaaaatcccgacgggtccaggattaaactccagctgacctcc

SEQ ID NO: 40 - DNA, 3162 bp, synthetic construct
  source 1..3162: mol_type = other DNA
atgtccagtgccattaagtcctataagtccgtgctgaggcccaacgaaaggaagaaccaactgcttaagtccaccatccagtgcttggaggacggatctgccttcttcttcaagatgctccaggggctctttggaggcatcacccccgagatcgtcaggttctccacagagcaggagaagcagcagcaggacatcgccctgtggtgtgccgtcaactggtttaggcccgtgtcccaggactcactgacccacaccatcgcctccgacaacctcgttgagaagttcgaagagtattacggcgggaccgcttccgacgctatcaagcagtacttctccgcctccataggtgagtcctattactggaacgattgcagacagcagtactacgacctctgcagggagctcggtgtcgaggtctccgacctcacccacgatcttgaaatcctttgcagggagaagtgcctggccgtcgccaccgaatctaaccaaaacaactccattatcagtgtgctgttcgggacaggggagaaggaggacaggtccgttaagttgaggatcaccaagaaaattctggaggccatctccaatctcaaggaaatcccaaagaacgttgcccccatccaagagatcatactcaacgtcgccaaggccacaaaagagacattcaggcaggtgtatgctggaaacctcggtgctccctccaccctggaaaagttcatagccaaggacggccagaaggagttcgatctgaagaagctgcagaccgacttgaaaaaggttatcaggggcaagtccaaggagagggactggtgttgccaggaggagctgaggagttacgtcgagcagaataccatccagtacgacctgtgggcatggggggagatgtttaacaaagcccacactgccctgaaaattaaatccaccagaaactacaatttcgccaaacagagactcgagcaatttaaggagatccaatccctgaacaacctcctggtggtgaagaagctgaacgattttttcgactcagaattcttctccggcgaggagacctacaccatctgcgtccaccaccttggcgggaaggacctctccaagctctacaaggcctgggaggacgaccctgccgaccccgagaatgccatcgttgtcctgtgtgacgacctcaaaaacaatttcaagaaggaacccatcagaaatatcctcaggtacatcttcaccatcaggcaggagtgtagtgcccaagacattttggctgccgccaaatataaccagcagctggataggtataaaagccaaaaagccaacccctccgtgctgggaaaccaaggcttcacttggaccaacgccgttatccttcctgaaaaggcccagaggaatgataggcccaattcccttgacctgaggatctggctgtatcttaagttgaggcaccccgacggtaggtggaaaaagcaccacatccccttttacgacaccaggttcttccaggagatctacgccgccgggaactcacccgtcgacacttgccaatttaggacccccaggttcggctaccacctgccaaagttgaccgaccagaccgccatcagggttaacaagaaacacgttaaagcagccaagaccgaggctagaatcaggctcgccatccagcaggggaccctccctgtctccaacctgaagatcaccgagatctccgcaactatcaactccaaaggacaggtcaggatccccgtcaagttcgacgtggggaggcagaagggtaccttgcagataggtgataggttctgtggctatgatcagaaccagaccgcatcacatgcatattccctttgggaggtggttaaagaggggcagtaccacaaggagctcggctgcttcgtgagattcatatcctctggggacatagtgtccatcacagagaacagggggaaccagttcgatcagctctcctacgaaggccttgcatacccacagtacgccgattggaggaaaaaggcctccaaattcgtctccctctggcagatcactaaaaagaataagaagaaggaaattgtcaccgttgaagccaaagagaagttcgacgcaatctgcaagtaccagcccagactgtacaaatttaataaagagtacgcctacctgctcagggatattgttaggggcaagtccctggtggaattgcagcagatcaggcaagaaatctttagattcatcgagcaggattgcggcgtgaccagactcgggtccctctccttgtccacactggagaccgtgaaagccgtcaagggcattatctactcttatttctccacagcactgaacgccagtaagaacaacccaatctccgatgaacaaaggaaagagtttgaccccgagctgttcgccctgctcgagaagcttgaattgattagaaccaggaagaagaagcagaaggtggagagaatagctaactcccttatccaaacttgcctcgagaataacataaagttcatcagaggtgagggggatctctccaccaccaacaacgccaccaagaagaaagccaactccaggtccatggactggctggccaggggggtttttaacaagatcaggcaactcgcccccatgcataacatcaccctcttcggatgcgggtccctctacacctcccatcaggaccctttggtccataggaaccccgacaaggccatgaaatgcaggtgggccgctatacccgtgaaggacatcggagactgggtcctcaggaagctctcccagaatctgagggccaagaatatcggcaccggggagtactatcaccagggggtgaaagagtttctgtcccattacgagctccaagacctggaggaagagctcctcaagtggagaagtgacaggaagtccaatattccctgctgggtgctgcagaacaggctggccgaaaagctcggcaacaaggaggccgtcgtgtacatccccgttagaggcgggaggatctacttcgccacccataaagttgccaccggcgccgtgtccatcgtcttcgaccaaaagcaggtctgggtctgcaacgccgaccacgtggccgccgcaaacatcgctctcactgttaaagggatcggcgagcagtcatccgacgaggagaaccccgacggctctagaattaaattgcaacttacctcc

SEQ ID NO: 41 - DNA, 3162 bp, synthetic construct
  source 1..3162: mol_type = other DNA
atgtcctccgccattaagtcctacaagtccgtgcttaggcccaacgagaggaaaaaccagctgctgaaatctaccattcaatgtctcgaggatgggtccgccttcttcttcaaaatgctgcagggactcttcgggggcatcacacccgagatagtcaggttctccactgagcaggagaagcaacagcaggacattgcactctggtgcgccgtcaactggtttaggcccgtcagtcaggactctctgacccacactatcgcctctgacaatctggttgagaagttcgaggaatactatggcggcaccgcctccgacgccatcaagcagtacttctccgcctccatcggtgagtcctactactggaacgactgtaggcagcagtactacgacctgtgtagagagctcggtgtggaagttagtgacttgacccatgatctcgaaatcttgtgcagggagaagtgtctcgccgtcgccaccgaatccaaccagaataatagtatcatctccgttctttttgggactggtgagaaggaggacaggtccgttaaactgaggatcaccaagaagatccttgaggctatctccaatttgaaagaaatccccaagaacgtggctcccatacaggagatcattctcaacgtggccaaggccactaaggaaaccttcaggcaagtctacgcaggcaacctcggcgccccctccaccctcgagaagttcatcgccaaggacggccaaaaggagttcgacctgaagaagctgcagaccgacctcaagaaagtcatcaggggcaagtccaaagagagggactggtgctgccaggaggagctgaggtcctatgtcgagcaaaacaccattcagtacgatttgtgggcctggggtgaaatgtttaacaaagcccataccgccctcaaaattaaatccaccaggaactataactttgccaagcagaggctcgagcaattcaaggagatccaatccctcaacaacctcctggtcgttaagaagcttaacgacttcttcgattctgaatttttctccggtgaagagacatacaccatatgcgtgcatcaccttggcgggaaggatctgtccaagctctacaaggcctgggaggacgaccccgccgatcccgagaacgcaatcgtggttctctgcgacgacctcaaaaacaactttaagaaagagcccatcaggaatatactcagatacatcttcaccatcaggcaggagtgctcagcccaggacatcctggccgctgccaaatataaccagcagttggacaggtataaaagccagaaggccaacccatcagtcttggggaatcaaggttttacctggaccaacgccgtcatcctgccagaaaaggctcaaaggaacgacagacccaactccctcgatctcaggatctggctgtatctgaagcttaggcaccctgacggcagatggaagaagcaccacatccccttctatgacaccaggttcttccaggagatatacgctgcagggaactcccctgtggacacctgccagttcagaacccctaggtttggctaccacctccccaagctcactgatcagacagccatcagggtcaacaaaaagcacgtgaaggccgccaagacagaggcaaggattagattggccattcagcagggaacactgcctgtctccaacctcaagatcaccgagatatccgccaccatcaactccaaggggcaggtcaggatccccgttaagttcgacgttgggagacagaaggggaccttgcagatcggcgacaggttctgcgggtacgatcagaaccagaccgcatcacacgcctacagtctttgggaagtggttaaagaggggcagtaccacaaggagcttggctgtttcgttaggttcatctcctccggggacatcgtttctattaccgagaacagggggaaccagttcgaccaactctcctacgagggactggcctacccacagtatgccgactggagaaagaaagcctccaagttcgtttccctctggcagatcaccaagaaaaacaagaagaaggagatcgtgaccgtcgaggccaaggagaagttcgacgccatctgcaagtatcagcccagactctacaagttcaacaaagaatacgcctacctgttgagggacatcgtgagaggtaaatcccttgtcgagctgcagcagatcaggcaggaaatatttagatttatcgagcaagattgcggggttaccaggctgggctccctgtccctctctaccctggagaccgtgaaggcagtcaagggaatcatctactcctacttctcaaccgccttgaatgcctccaagaataacccaatctccgacgaacaaaggaaggagttcgaccccgagctgttcgccttgctggagaagctggagctcatcaggaccaggaagaagaaacagaaggttgaaagaatcgccaattccctcatccagacctgcctggagaacaatatcaagttcatcaggggcgagggcgacctctccaccaccaacaatgccaccaagaagaaagccaactccagatccatggactggctcgccaggggggtgtttaacaagatcaggcagctcgctcccatgcacaacatcacactgttcgggtgcggctccctttatacctcccaccaggaccccctcgtgcacaggaaccccgataaggccatgaaatgcaggtgggccgccatccccgtgaaagatataggggattgggtgctgaggaagctgtcccaaaatctgagggcaaagaacataggaactggcgagtactatcatcaaggggtgaaagagttcctctcccactacgagctgcaggacctcgaagaggagttgcttaaatggagatccgacaggaagtccaacatcccctgctgggtcctgcaaaacagactggcagaaaaactgggcaataaggaggccgtcgtgtacatccccgtgaggggggggaggatttacttcgctacacataaagtggccaccggcgccgtgtccatcgtttttgaccagaaacaggtgtgggtctgcaacgccgaccatgtcgccgccgctaacatcgctctcaccgtcaagggcatcggagaacagtccagtgatgaggagaatcccgacggatcaaggattaaacttcaactgacttcc

SEQ ID NO: 42 - DNA, 3162 bp, synthetic construct
  source 1..3162: mol_type = other DNA
atgtcctcagccatcaagagttataagtccgtgctcaggcccaatgagagaaaaaatcaactcctcaagtccaccatccaatgtctggaggatgggagtgcctttttcttcaagatgttgcagggtttgttcgggggtatcacccccgaaatagttagattctccaccgaacaagagaagcagcagcaggacatcgccctctggtgcgcagtcaactggtttagaccagtgtcccaagactccctcacacacaccatcgccagtgataacctggttgagaagttcgaggagtactacggcggcaccgcctccgacgctatcaaacagtatttcagtgcctccataggcgagtcctactactggaatgattgtagacagcagtactacgacctttgtagagagctgggtgttgaggtgtccgacctgacacacgacctggaaattctttgcagagagaagtgcctcgccgtcgcaaccgagtccaaccagaataactctatcatctccgttctctttggaaccggagagaaggaggacaggtccgtcaagcttagaatcaccaagaagatcctcgaggccatctccaacctgaaggagattcccaaaaacgtcgctcccatccaggagatcatacttaacgtcgctaaggccaccaaagaaacctttaggcaggtttacgccggcaacttgggggccccatcaactctcgaaaagtttatcgccaaggatggccagaaggaattcgacctcaagaagctccagaccgatctgaagaaggtgatcagggggaagtcaaaagagagagactggtgctgccaggaagagttgaggtcatatgtggaacagaacacaatccagtacgatctctgggcctggggggaaatgttcaacaaggcccacaccgccttgaaaattaaatccaccagaaattataacttcgccaagcagaggctcgagcagtttaaggaaatccaatccctgaacaatctcctcgtcgtcaagaagctcaacgactttttcgactccgaattcttctccggtgaggaaacctacaccatctgtgttcaccatctggggggaaaggacctgtccaagctgtacaaagcctgggaggacgatcccgctgaccccgagaacgcaatcgtcgtcctctgcgacgatctcaaaaacaatttcaaaaaagagcccatcaggaacatcctcaggtacatattcacaatcaggcaggagtgctccgcccaagacattctggccgccgccaaatataaccagcagctcgacaggtataaaagccaaaaggccaacccttccgtgctgggcaaccaaggattcacttggactaacgccgttatactgcccgaaaaggcccaaaggaacgacaggccaaacagtctggatctgaggatctggctgtaccttaagctgaggcaccccgacgggaggtggaagaaacatcacatccctttctacgacaccaggttttttcaggagatttatgcagccgggaattcccccgtggacacctgccagttcagaacccctagattcgggtatcaccttcccaagctcacagaccagaccgccatcagggtgaataagaagcacgtcaaggctgccaaaaccgaggccaggatcaggctggccatccagcagggcaccctccccgtgtccaatctcaagatcactgagatctctgcaaccattaattctaaggggcaggttaggatccccgtgaaattcgacgttggcaggcagaagggcaccctgcaaatcggggacaggttctgcgggtacgaccagaaccaaaccgcctcccacgcctactccctctgggaggttgtcaaggagggacagtaccataaggagctgggttgttttgtgaggttcatctcctccggagatatcgtgtccatcacagagaacagggggaaccagttcgatcaactcagttacgagggcctcgcctacccccagtacgccgactggaggaagaaggcctccaagttcgtgtctctgtggcaaatcaccaagaaaaacaaaaagaaggagatcgttaccgtcgaggccaaggaaaaattcgacgccatctgcaagtaccagcccagactgtacaaattcaacaaagaatacgcctacctcctgagagacatcgtgaggggtaagtccctcgtcgaattgcagcagattaggcaagagatcttcaggttcatcgagcaggactgcggcgtgaccaggctggggtccctgtccctttcaaccctggagaccgttaaggccgtcaagggcataatatactcctatttctccaccgccctcaacgcctccaagaacaaccccatttccgatgagcaaaggaaggagttcgaccccgagctcttcgctctcctggagaagctcgagcttattagaaccaggaagaagaaacagaaggtcgagaggatcgccaactccttgatccagacctgcctcgagaataacatcaaatttatcaggggcgagggggacttgtccaccaccaacaatgccacaaagaagaaggcaaactccaggtccatggactggctggccaggggggtgtttaacaaaataaggcagctggcccccatgcacaacatcacccttttcgggtgcggatccctgtacacctcccaccaggaccccctggtccataggaaccccgacaaagccatgaaatgcaggtgggcagccatacccgtcaaggacatcggcgactgggtgttgaggaaactctcccagaaccttagggccaagaacattggcactggggaatactaccatcagggcgtcaaggagttcctttcccactatgagcttcaggacctggaggaagagctgttgaagtggaggtccgataggaagtccaacatcccttgctgggtcttgcaaaatagattggccgagaagctgggcaacaaggaggccgttgtctatatccccgtgaggggcggcaggatctacttcgctacccataaagtcgctactggtgccgtgtccatcgttttcgatcagaaacaggtgtgggtttgcaacgctgaccacgtggcagcagccaacatcgccctcaccgtgaagggcatcggggagcagtcttccgacgaggagaacccagacgggtccaggattaaactgcagctcacttcc

SEQ ID NO: 43 - DNA, 3162 bp, synthetic construct
  source 1..3162: mol_type = other DNA
atgtcctccgcaataaaatcctacaagtccgttctgaggcccaacgagaggaagaatcaactgctcaaatccacaattcaatgcctcgaggacgggtccgccttcttttttaaaatgctgcagggtctgtttggcggcatcacccccgagatcgtcaggttctccaccgagcaagagaagcagcagcaggacatcgccctgtggtgcgccgtcaactggtttaggcccgtctcccaggattcactcacacacaccatcgcttccgacaaccttgtggagaagtttgaagaatactacgggggtaccgcctccgatgcaatcaagcagtatttcagtgcctccatcggcgagtcttattattggaatgattgcaggcagcaatactacgacctgtgcagggagctcggagtcgaagtttctgaccttacccacgatctggagatcctgtgtagagagaagtgcctcgcagttgccaccgagtccaaccagaacaactccatcatctccgtcctcttcgggaccggcgagaaggaggataggtctgtgaagctcaggattaccaagaagatcctggaagctatctccaatctcaaggaaatccccaagaacgtggcccccatccaggagataatccttaatgttgccaaggccaccaaggaaactttcaggcaggtctacgcaggaaacctcggggccccctccaccctggagaagttcatagccaaggatggccagaaggagttcgacctgaaaaaacttcaaacagatttgaagaaggtcattaggggtaagtccaaggaaagggactggtgctgccaggaggagctgagatcctacgtcgagcagaacaccatccaatacgacttgtgggcctggggggaaatgttcaacaaagctcacaccgccctgaaaattaaatctaccaggaattacaatttcgccaaacagagactggagcagtttaaggagattcaatcccttaacaatctcctcgtggtcaagaaacttaacgacttcttcgactccgagtttttcagtggtgaggaaacatacaccatctgcgtgcatcatctcgggggaaaggacctgtccaagctctacaaggcctgggaggacgaccccgccgacccagaaaatgccatcgtcgtgctgtgtgacgaccttaagaacaactttaagaaggagcccatcaggaacatcttgaggtacatttttaccatcagacaggagtgctccgcccaggacatcctggccgccgctaaatataaccagcaacttgacagatataaaagccagaaggccaacccatcagttctcgggaaccagggctttacatggaccaacgccgttatactccccgaaaaggcccagagaaatgataggcctaactccctggacttgaggatatggctttacctgaaactcaggcaccctgatgggaggtggaaaaagcaccacattccattctacgacacaaggttcttccaggaaatttacgccgccggcaactcccccgttgacacctgccaattcaggacccccaggttcggctaccacttgcccaagttgaccgaccagaccgcaatcagggtcaacaagaagcacgttaaggccgcaaagaccgaggctagaatcagactcgccatccagcagggaaccttgcccgtctccaacctgaaaatcaccgagataagtgccaccatcaactccaagggccaggtcaggataccagtgaaattcgatgtcggcaggcagaagggcacactccagattggggataggttctgcggttacgaccagaaccagaccgcctcccacgcctactcactttgggaggtggtcaaggaggggcaatatcataaggagctcggttgcttcgtgaggttcatttcctccggagacatcgtgtccatcaccgagaacagggggaatcagttcgaccagctgtcctatgagggcctcgcatacccccaatacgccgattggaggaagaaagcctccaagttcgtttccctctggcaaatcaccaagaaaaacaagaagaaagagatcgtcaccgtggaggccaaagagaagttcgacgctatatgcaaatatcagcccaggctctacaagttcaacaaggagtacgcctatctgctcagggacatcgtgagaggcaagtcactcgtcgagctgcagcagatcagacaagagatcttcaggtttatcgaacaggactgtggggtgaccagactgggctccctctctctgtccaccctggaaaccgtgaaggctgtcaaaggcatcatctattcctacttctccactgccctgaacgcctccaagaacaaccccatcagtgacgagcagaggaaagagttcgaccccgagctcttcgccctcttggagaagttggagctcatcaggaccaggaagaaaaagcagaaggttgagaggatcgccaactccctcatccagacttgcctggagaacaatatcaaattcatcaggggtgagggcgacttgtccactaccaacaacgcaactaagaagaaggccaactccaggtccatggactggctggccaggggagttttcaataagatcagacagctggctcccatgcacaacatcaccctgttcggctgtggctccctctacacctcccaccaggatcccctcgtccacagaaaccctgacaaggccatgaaatgcaggtgggccgccatccccgtgaaggacataggggactgggtgctcaggaagttgtcccagaacctgagggctaagaatatcggaaccggcgagtattaccatcaaggggtgaaagagttcctgtcccactacgaactccaggacttggaggaagagctgcttaagtggaggtccgacaggaaatcaaacatcccctgctgggttcttcaaaacagactcgcagagaagctggggaacaaggaagccgtggtttacatccccgtgagggggggcagaatctacttcgccactcataaagtcgccaccggggccgtttccatcgtgtttgaccagaagcaggtctgggtctgtaacgccgaccatgtggccgccgccaatatcgccctcactgttaaggggatcggggaacaatccagtgacgaggagaaccctgatgggagtaggattaaactgcagctcacctcc

SEQ ID NO: 44 - DNA, 3162 bp, synthetic construct
  source 1..3162: mol_type = other DNA
atgtcctctgctattaagtcctacaaatccgttctgagacctaacgagaggaagaaccagttgctgaagtccaccatccagtgcctggaggacgggtccgccttcttcttcaagatgctgcagggcctgtttgggggtatcacccccgaaatcgttaggttctccacagaacaggaaaagcaacagcaagacatcgccctgtggtgtgcagtgaactggttcaggcctgtgtcccaggactccctgacccacactatagcctccgacaatctcgtcgagaaattcgaggagtactacggcggaactgcttccgatgccatcaaacagtacttctccgcctccatcggtgaatcctactactggaatgactgcaggcagcagtattacgacctctgtagggaactcggggtcgaggtgtccgacctgacacacgatctggagatcctgtgcagagagaagtgccttgccgtggccacagagtccaaccagaacaactccataatatccgtgctttttgggaccggagagaaggaggacaggtccgtcaaactcaggatcaccaaaaagatcctcgaggcaatctcaaaccttaaagagatcccaaagaatgtggcccccatccaagagatcatcctgaacgtggcaaaggccaccaaggaaacctttaggcaggtctatgctggcaatctgggggccccctccaccttggagaagtttatcgctaaggacggccagaaagagttcgacctcaagaagctgcagaccgatctcaagaaggtcataagggggaaatctaaggaaagagactggtgctgccaggaggaactcagatcctacgttgagcagaacaccattcagtatgacctgtgggcatggggagagatgttcaacaaggcccacaccgccctgaaaattaaatctaccaggaactacaacttcgccaagcagaggctggaacaatttaaggagatccagtccctcaataacctgctcgtcgttaagaagcttaatgactttttcgactccgagttcttctccggcgaagagacatataccatctgcgtccaccacctcggagggaaggacctctccaaactgtataaagcctgggaggatgaccccgctgatcccgagaacgctatagtcgtcctgtgcgacgatttgaaaaacaacttcaagaaggagcccatcagaaatattctcaggtatatcttcaccattaggcaggaatgttccgcccaagacatcttggccgccgccaaatataaccagcagctggatagatataaaagccaaaaggccaacccctccgtgttgggaaaccagggctttacctggaccaacgccgttatcctccccgagaaggcccaaaggaatgacaggcccaactccctcgatctgaggatatggctttacctcaagctgaggcatcctgatggtaggtggaaaaaacaccacatcccattctacgacactaggtttttccaggagatctatgccgccggcaactctcccgtggacacctgccagttcaggacccccaggttcgggtaccacctgcccaagctgactgaccagaccgccattagggtcaacaaaaagcatgtcaaggccgccaagaccgaggccaggatcaggctggccattcagcagggcaccctccccgtctccaacctcaagatcaccgagatctccgccactatcaattccaaggggcaggtcaggatcccagtgaagttcgacgtgggaaggcagaaggggaccctgcaaataggcgacagattctgcgggtacgaccaaaaccagactgcctcccatgcctactccctctgggaggttgtgaaagagggccagtaccataaggagctcggttgcttcgttagattcatctcctccggcgacatcgtttccatcaccgaaaacagaggcaatcagttcgaccagctgagttacgagggcctcgcctaccctcaatacgccgattggaggaagaaggcatccaagtttgtctccctttggcagatcaccaagaagaacaagaagaaggagatagtgactgttgaggctaaggaaaagttcgatgccatctgcaaataccagcccagactgtataagttcaacaaagagtacgcatacctcctgagggacatcgttaggggaaaatccctcgtggaactccagcagatcaggcaagagatcttcaggttcatcgagcaagattgcggggttaccagacttgggagtctctccctttcaaccctggaaaccgtcaaagccgtcaaggggatcatctactcctacttctccaccgccctcaatgccagtaaaaacaaccccatttccgacgagcagaggaaagaattcgaccccgagttgtttgccttgctcgagaagttggagttgatcaggaccaggaagaagaagcagaaagttgagaggatcgccaactccttgatccagacctgcctcgagaacaatataaagttcatcaggggcgagggcgacctcagtaccacaaacaacgccaccaagaaaaaggccaattccagatccatggactggttggccagaggcgtttttaataagatcaggcagctcgcccccatgcataacatcacacttttcgggtgtgggtcattgtatacctcccaccaggaccccctggtccacaggaaccccgacaaggccatgaaatgtaggtgggctgccattcccgtgaaggatatcggcgactgggtgctgaggaagctttctcaaaacctgagggcaaagaacattggcaccggggagtactaccaccaaggtgtgaaggagttcctcagtcactacgaactccaagacctggaggaggagctccttaagtggagatccgacaggaagtccaacatcccctgctgggtgctccagaacaggctcgccgagaagcttggcaacaaggaggccgtggtctacatcccagtgaggggcgggaggatctactttgccacacataaagtggccaccggtgccgtctcaatcgtcttcgatcagaagcaagtttgggtttgcaacgcagaccacgtcgccgctgcaaacatcgccctcaccgtcaaggggatcggtgagcagagttccgacgaggaaaaccccgacgggtccagaattaaactgcagctgacctca

(19) United States
(12) Patent Application Publication
(10) Pub. No.: US 2025/0154518 A1
(43) Pub. Date: May 15, 2025

(54) USE OF CAS12F NUCLEASES IN PRODUCTION OF EXPRESSION MODULATED PLANT MATERIALS

(71) Applicant: INARI AGRICULTURE TECHNOLOGY, INC., Cambridge, MA (US)

(72) Inventors: Michael Andreas Kock, Rheinfelden (DE); Thomas Dubois, Cambridge, MA (US)

(21) Appl. No.: 18/835,223

(22) PCT Filed: Feb. 7, 2023
(86) PCT No.: PCT/US2023/062089

§ 371 Date: Aug. 1, 2024

Related U.S. Application Data
(60) Provisional application No. 63/267,643, filed on Feb. 7, 2022.

(51) Int. Cl. C12N 15/82; C12N 9/22; C12N 15/11; C12Q 1/6895

(52) U.S. Cl. CPC C12N 15/8213; C12N 9/22; C12N 15/11; C12Q 1/6895; C12N 2310/20; C12N 2310/531; C12Q 2600/13

(57) ABSTRACT

Methods for obtaining plant cells, plants, and plant parts, including soybean and corn plant cells, comprising Cas12F nucleases and at least one guide RNA that target the DNA regulatory sequences including a promoter region, thereby producing a gene allele with altered expression levels. The methods can be used to generate alleles of genes of interest by selecting for phenotypes conferred by the altered expression of the gene(s). Also provided are soybean plant cells and com plant cells comprising synthetic polynucleotides that provide for gene alleles with altered expression levels.

### CROSS REFERENCE TO RELATED APPLICATIONS

This international patent application claims benefit of provisional patent application U.S. Ser. No. 63/267,643, filed Feb. 7, 2022. The U.S. provisional patent application is herein incorporated by reference in its entirety, including without limitation, the specification, claims, and abstract, as well as any figures, tables, appendices, or drawings thereof.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Jan. 31, 2023, is named “P13711WO00_SequenceListing.xml” and is 163,811 bytes in size.

### FIELD

The disclosure is generally related to methods, cells, and compositions for producing a gene allele with altered expression levels in plant cells and plant parts, including soybean and corn plant cells and plant parts.

### BACKGROUND

The CRISPR/Cas system of bacterial acquired immunity against phages and viruses has been adapted into potent new technologies for genomic modifications and gene expression control.

The CRISPR/Cas9 system is the most commonly used type II CRISPR system. It recognizes the PAM motif of 3′-NGG and cuts the target sequence with blunt ends. The CRISPR/Cas Type V system is a type of CRISPR system that has a 5′-TTN motif and cuts target sequences with sticky ends, such as Cpf1, C2c1, CasX, and CasY.

However, many CRISPR/Cas systems suffer from several disadvantages including the requirement to use multiple guide RNAs (gRNAs) to make deletions. For example, the need for multiple guide RNAs to direct Cas nucleases to a promoter or other non-coding region targeted for Cas nuclease-mediated editing is problematic, since cutting efficiencies between different gRNAs differ, and cuts at different sites need to be simultaneous in some cases.

European Patent Application No. 3875469 A1 discloses a Crispr/Cas12F enzyme and method for nucleic acid editing.

### SUMMARY

Methods of obtaining plant cells with altered expression of at least one gene comprising contacting at least one DNA regulatory sequence (e.g., a promoter region) of the gene in the genome of the plant cell with a Cas12F nuclease and at least one guide RNA that target the DNA regulatory sequence (e.g., a promoter region) are provided. In embodiments, the method further includes selecting a plant cell, plant callus, plant part, or plant comprising at least one first deletion in the DNA regulatory sequence (e.g., a promoter region) of the gene that confers the altered expression of the gene.

Also provided are soybean plant cells comprising a DNA molecule encoding a Cas12F nuclease, wherein the DNA molecule has: (i) a GC (guanine and cytosine) content greater than 47 or 48% and/or (ii) a melting temperature (Tm) greater than 89 or 90 degrees Celsius.

Also provided are soybean plant cells comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.

Also provided are corn plant cells comprising a synthetic DNA molecule having at least 85%, 90%, 95%, 98%, 99%, or 100% sequence identity to SEQ ID NO: 4, SEQ ID NO: 11, or SEQ ID NO: 12 is provided.

Also provided is a corn plant cell comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.

### BRIEF DESCRIPTION OF THE DRAWINGS

FIG. 1 shows the mature direct repeat part of a guide RNA (“gRNA”) modeled secondary structure for Cas12f.4 gRNA (left-most; comprising SEQ ID NO: 6 and four 3′ terminal “N” nucleotides), Cas12f.5 gRNA (middle; comprising SEQ ID NO: 7 and three 3′ terminal “N” nucleotides), and Cas12f.6 gRNA (right-most; comprising SEQ ID NO: 8 and three 3′ terminal “N” nucleotides), respectively.

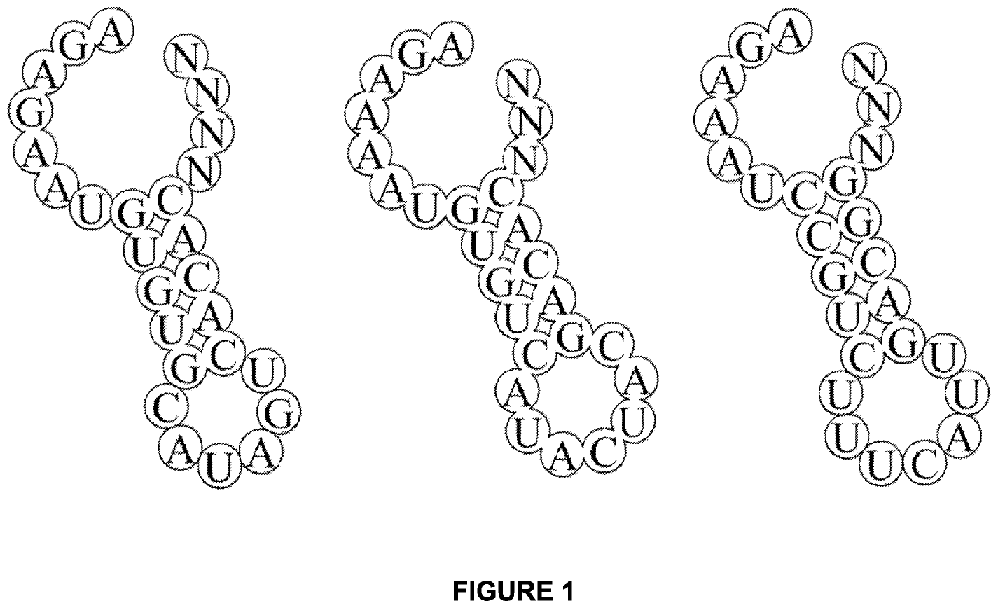

FIG. 2 shows the distribution of deletion sizes at edited target sites for Cas12f.4 (top) and control (bottom). The data represent 90 samples from soybean and corn Cas12f.4 editing tests, and 500 control samples. The distributions show that RNA/Cas12f.4 induced larger deletions in corn (top) than to soybean (bottom). Cas12f.4 included deletions with a size distribution of 1, 7, 10, 12, and 34 nucleotides at the respectively minimum, 25th percentile, median, 75th percentile, and maximum points of the range. The corresponding control distribution of deletion size values was at 1, 3, 7, 10, and 53 nucleotides. Deletion edits made up about 95% of all edits for both Cas12f.4 and controls.

FIG. 3 shows the median and Interquartile Range (IQR) of deletion sizes in corn (“maize”) (left) and soybean (right) caused by test Cas12f.4 nucleases (left-most) and control nucleases (right-most). The distribution of the IQRs shows that Cas12f.4 test nucleases produce longer deletions more frequently than control nucleases in both corn and soybean.

### DETAILED DESCRIPTION

### Definitions

The singular forms “a,” “an,” and “the” can include plural referents unless the content clearly indicates otherwise.

The term “and/or” where used herein is to be taken as specific disclosure of each of the two or more specified features or components with or without the other specified features. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “alleles” refers to alternate forms of a DNA sequence at a genetic locus, that is, a position on a chromosome of a gene or other chromosome marker.

As used herein, the terms “correspond,” “corresponding,” and the like, when used in the context of an amino acid position, mutation, and/or substitution in any given Cas12F nuclease with respect to the reference Cas12F nuclease, all refer to the position, mutation, and/or substitution of the amino acid residue in the given Cas12F nuclease sequence that has identity or similarity to the amino acid residue in the reference polypeptide sequence when the given Cas12F nuclease polypeptide sequence is aligned to the reference Cas12F nuclease polypeptide sequence using a pairwise alignment algorithm (e.g., CLUSTAL O 1.2.4 with default parameters).

As used herein, a “frameshift mutation” refers to a mutation in a nucleic acid sequence that results in the wild-type reading frame being shifted to different reading frame such that translation of an mRNA having a frameshift mutation results in a departure from the wild-type reading frame.

As used herein, the phrase “nonsense mutation” refers to any mutation that results in the appearance of a stop codon where previously there was a codon specifying an amino acid. The presence of this premature stop codon results in the production of a truncated protein.

“Heterologous,” as used herein, means a nucleotide or polypeptide sequence that is not found in the native nucleic acid or protein, respectively. For example, relative to a native Cas12F nuclease polypeptide, a heterologous polypeptide comprises an amino acid sequence from a protein other than the Cas12F nuclease polypeptide.

As used herein, the term “amorphic allele” refers to an allele of a gene having no gene activity in comparison to the wild-type allele of the gene. Amorphic alleles are also known as null alleles.

As used herein, the term “isomorphic allele” refers to an allele of a gene having wild-type gene activity.

As used herein, the term “hypomorphic allele” refers to an allele of a gene having reduced or partially reduced but not null gene activity in comparison to the wild-type allele of the gene. In embodiments, a hypomorphic allele is an allele that results in gene activity levels that are at least 20% lower (e.g., at least 20%, at least 30%, at least 40%, at least 50%, at least 60%, at least 70%, at least 80% or at least 90% lower) than gene activity levels of an allele of the gene that does not contain the mutation (e.g., a wild-type allele). In embodiments, a hypomorphic allele is an allele that results in an expression levels of a gene that are at least 20% lower (e.g., at least 20%, at least 30%, at least 40%, at least 50%, at least 60%, at least 70%, at least 80% or at least 90% lower) than expression levels of an allele of the gene of interest that does not contain the mutation (e.g., a wild-type allele).

As used herein, “gene activity” comprises any measure of gene function. Gene activity measures thus include measures of gene-mediated phenotypes, gene-encoded RNA expression, gene-encoded RNA activity, gene-encoded protein expression, and/or gene-encoded protein activity (e.g., enzymatic or other biochemical activity).

As used herein, the terms “include,” “includes,” and “including” are to be construed as at least having the features to which they refer while not excluding any additional unspecified features.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “naturally-occurring” as used herein as applied to a nucleic acid, a protein, a cell, or an organism, refers to a nucleic acid, cell, protein, or organism that is found in nature.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

As used herein, the term “exogenous nucleic acid” refers to a nucleic acid that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or with introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may indeed act to modulate production of a desired product by various mechanisms (see “DNA regulatory sequences,” below).

Thus, e.g., the term “recombinant” polynucleotide or “recombinant” nucleic acid refers to one which is not naturally-occurring, e.g., is made by the artificial combination of two otherwise separated segments of sequence through human intervention. An artificial combination can be made by methods which include chemical synthesis, biochemical synthesis (e.g., by a polymerase chain reaction), and/or the artificial manipulation of isolated segments of nucleic acids, e.g., by genetic engineering techniques.

Similarly, the term “recombinant” polypeptide refers to a polypeptide which is not naturally-occurring, e.g., is made by the artificial combination of two otherwise separated segments of amino sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific polynucleotide sequence(s), or is to be used in the construction of other recombinant polynucleotide sequences.

The term “DNA regulatory sequence(s),” as used herein refers to any gene control sequence (e.g., a promoter, transcriptional enhancer, transcription initiation site, 5′ untranslated region, intron, 3′ untranslated region, polyadenylation signal, terminators, encoded RNA degradation signal, encoded RNA targeting signal, an encoded protein degradation signal, and/or an encoded protein targeting signal).

The phrase “target site” as used herein refers to any or all of the polynucleotide sequences: (i) that can be or are bound by a Cas nuclease or Cas nuclease complexed with a guide RNA; and/or (ii) that comprise an endonuclease or nickase cleavage site of a Cas nuclease or Cas nuclease complexed with a guide RNA. In certain embodiments, the Cas nuclease is a Cas12F nuclease.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (e.g., in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell of a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Examples of conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990),/.Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See/.Mol. Biol. 48:443-453 (1970). CLUSTAL and MUSCLE are other commonly used alignment programs.

As used herein, “Tm” is the melting temperature of the double stranded DNA sequence calculated using the following formula:

$$Tm ( ° C . ) = 7.35 \times E ) + [ 17.34 \times ln ( Len ) ] + [ \text{} 4.96 \times ln ( Conc ) ] + [ 0.89 \times ln ( DNA ) ] - 25.42$$

where Tm=Predicted melting temperature; E=DNA strength parameter per base=Cumulative DNA strength parameter/length of DNA sequence; Len=Length of nucleotide sequence (number of base pairs); Conc=[Na⁺] concentration of the solution (Molar)=0.16M; DNA=total nucleotide strand concentration=0.0001 g/mL, according to the method of Khandelwal G, Bhyravabhotla J (2010) PLOS ONE 5(8): e12433. doi.org/10.1371/journal.pone.0012433.

In the present disclosure, the “Cas12F” or “Cas12F nuclease” which has an amino acid sequence selected from the following: (i) a sequence as shown in any one of SEQ ID NOs: 1, 2, or 3; (ii) compared with the sequence as shown in any one of SEQ ID NOs: 1, 2, 3, a sequence having one or more amino acid substitutions, deletions or additions in SEQ ID NOs: 1, 2, 3 (for example, 1, 2, 3, 4, 5, 6, 7, 8, 9 or 10 amino acid substitutions, deletions or additions); or (iii) a sequence having at least 20%, at least 30%, at least 40%, at least 50%, at least 60%, at least 70%, at least 80%, at least 80%, at least 85%, at least 90%, at least 91%, at least 92%, at least 93%, at least 94%, at least 95%, at least 96%, at least 97%, at least 98%, or at least 99% sequence identity with the sequence as shown in any one of SEQ ID NOs: 1, 2, or 3.

The Cas12F of the present disclosure is an endonuclease that binds to and cleaves a specific site of a target sequence under the guidance of a guide RNA, and has DNA and RNA endonuclease activities at the same time.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Description

Information on partial sequences involved in the present disclosure is provided in Table 1 below.

TABLE 1
Description of the sequence

| SEQ ID | | Sequence Type |
|---|---|---|
| NO: | Description | DNA/PRT/RNA |
| 1 | Synthetic Cas12f.4 | PRT |
| 2 | Synthetic Cas12f.5 | PRT |
| 3 | Synthetic Cas12f.6 | PRT |
| 4 | Synthetic Corn Cas12f.4 | DNA |
| 5 | Synthetic Soy Cas12f.4 | DNA |
| 6 | Cas12f.4 Synthetic gRNA | RNA |
| 7 | Cas12f.5 Synthetic gRNA | RNA |
| 8 | Cas12f.6 Synthetic gRNA | RNA |
| 9 | Synthetic Soy Cas12f.5 | DNA |
| 10 | Synthetic Soy Cas12f.6 | DNA |
| 11 | Synthetic Corn Cas12f.5 | DNA |
| 12 | Synthetic Corn Cas12f.6 | DNA |
| 13 | Synthetic Corn Cas12f.5 | DNA |
| 14 | Synthetic Corn Cas12f.5 | DNA |
| 15 | Synthetic Corn Cas12f.5 | DNA |
| 16 | Synthetic Corn Cas12f.5 | DNA |
| 17 | Synthetic Corn Cas12f.5 | DNA |
| 18 | Synthetic Corn Cas12f.5 | DNA |
| 19 | Synthetic Corn Cas12f.5 | DNA |
| 20 | Synthetic Corn Cas12f.5 | DNA |
| 21 | Synthetic Soy Cas12f.5 | DNA |
| 22 | Synthetic Soy Cas12f.5 | DNA |
| 23 | Synthetic Soy Cas12f.5 | DNA |
| 24 | Synthetic Soy Cas12f.5 | DNA |
| 25 | Synthetic Soy Cas12f.5 | DNA |
| 26 | Synthetic Soy Cas12f.5 | DNA |
| 27 | Synthetic Soy Cas12f.5 | DNA |
| 28 | Synthetic Soy Cas12f.5 | DNA |
| 29 | Synthetic Corn Cas12f.6 | DNA |
| 30 | Synthetic Corn Cas12f.6 | DNA |
| 31 | Synthetic Corn Cas12f.6 | DNA |
| 32 | Synthetic Corn Cas12f.6 | DNA |
| 33 | Synthetic Corn Cas12f.6 | DNA |
| 34 | Synthetic Corn Cas12f.6 | DNA |
| 35 | Synthetic Corn Cas12f.6 | DNA |
| 36 | Synthetic Corn Cas12f.6 | DNA |
| 37 | Synthetic Soy Cas12f.6 | DNA |
| 38 | Synthetic Soy Cas12f.6 | DNA |
| 39 | Synthetic Soy Cas12f.6 | DNA |
| 40 | Synthetic Soy Cas12f.6 | DNA |
| 41 | Synthetic Soy Cas12f.6 | DNA |
| 42 | Synthetic Soy Cas12f.6 | DNA |
| 43 | Synthetic Soy Cas12f.6 | DNA |
| 44 | Synthetic Soy Cas12f.6 | DNA |

Many CRISPR/Cas systems suffer from several disadvantages including the need for multiple guide RNAs to direct Cas nucleases to DNA regulatory sequences located in a given region targeted for deletion mutagenesis. This is problematic because cutting efficiencies between guide RNAs differ, and because cuts at different sites need to be simultaneous in some cases. The present disclosure provides Cas12F nucleases (e.g., Cas12f.4 nuclease, Cas12f.5 nuclease, Cas12f.6 nuclease, and/or engineered Cas12F nucleases) that can be used to address this problem (e.g., in the context of generating genetic diversity in plants and other target organisms).

In one embodiment, the present disclosure provides Cas12F nucleases (e.g., Cas12f.4 nuclease, Cas12f.5 nuclease, and Cas12f.6 nuclease) that can induce deletions in a target site significantly longer than those seen with Cas9 or other types of Cas12 nucleases when used with one or more gRNAs. In one embodiment, a Cas12F nuclease used with a one gRNA can induce deletions in the 10-20 nucleotide range at a target site recognized by the Cas12F nuclease and gRNA. In another embodiment, a Cas12F nuclease used with one gRNA can induce deletions in the 1-75 nucleotide range. In some embodiments, deletions of any one of 1, 2, 3, 4, or 5 to any one of 8, 10, 12, 14, 16, 18 or 20 base pairs of DNA can be induced at a target side recognized by a Cas12F nuclease and one gRNA. In some embodiments, deletions of any one of 5, 6, 8, or 10 to any one of 12, 14, 16, 18, 20, 22, 24, 26, 28, or 30 base pairs of DNA can be induced at a target side recognized by a Cas12F nuclease and one gRNA. In some embodiments, deletions of varying size can be induced at a target side recognized by a Cas12F nuclease and one gRNA, including, for example, deletions of 1 to 20 base pairs of DNA, 1 to 10 base pairs of DNA, 5 to 14 base pairs of DNA, 8 to 17 base pairs of DNA, 11 to 20 base pairs of DNA, 1 to 7 base pairs of DNA, 5 to 10 base pairs of DNA, 8 to 13 base pairs of DNA, 11 to 16 base pairs of DNA, 15 to 20 base pairs of DNA, or more than 20 base pairs of DNA (e.g., 20, 22, or 24 to 16, 18, 30, 32, 34, 38, 40, 45, 50, 55, 60, 65, 70, or 75 base pairs of DNA). In some embodiments, a Cas12F nuclease and gRNA are directed to induce deletions in various DNA regulatory sequences (e.g., promoter regions).

In one embodiment, the disclosure provides methods and plant cells (e.g., corn and soybean plant cells) that use or comprise Cas12F nucleases (e.g., Cas12f.4 nuclease, Cas12f.5 nuclease, and Cas12f.6 nuclease) that do not require multiple guide RNAs to induce deletions in target sites in DNA regulatory sequences (e.g., promoter regions). In one embodiment, a Cas12F nuclease can be directed to a DNA regulatory sequence (e.g., a promoter) in a given target gene with either one guide RNA or multiple guide RNAs simultaneously. In one embodiment, a Cas12F nuclease can be simultaneously directed to a DNA regulatory sequence (e.g., a promoter) in a first target gene with one guide RNA and to a DNA regulatory sequence (e.g., a promoter) in at least one additional target gene with another guide RNA. Guide RNAs used with the Cas12F nucleases can in certain embodiments comprise single gRNAs which comprise a direct repeat recognized by the Cas12F nuclease and a targeting sequence that is complementary to target site DNA located adjacent to a protospacer adjacent motif (PAM) site in the target site DNA. In embodiments, the single guide RNA can comprise a direct repeat which comprises SEQ ID NO: 6, 7, 8, or a variant thereof recognized by a Cas12F nuclease. In general, the targeting sequence of the guide RNA typically comprises about a 17, 18, 19, 20, 21, 22, 23, or 24 nucleotide sequence which corresponds to the sequence immediately adjacent to the 3′ end of a PAM or that has sufficient sequence identity to the target site to allow gRNA annealing and cleavage. In certain embodiments, the targeting sequence of the guide RNA typically comprises about a 17, 18, or 19 to about a 20, 21, 22, 23, or 24 nucleotide sequence which corresponds to the sequence immediately adjacent to the 3′ end of a PAM or that has sufficient sequence identity to the target site to allow gRNA annealing and cleavage. Examples of suitable methods of obtaining or designing guide RNAs suitable for use with certain Cas12F nucleases include those disclosed in European Patent Application No. 3875469. Other examples of methods for obtaining or designing Cas nuclease guide RNAs suitable that can be adapted for use with certain Cas12F nucleases include methods set forth in U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety. In some embodiments, the ability to use one guide RNA results in a higher efficiency of producing a plant cell with phenotype-producing deletions, since there is no need for paired guides to work in the same plant cell at the same time to produce the deletion. In addition, however, the ability to use the Cas12F nucleases with multiple guide RNAs enhances the likelihood of generating heterozygotes with combined different hypomorphic alleles.

In some embodiments, the disclosed Cas12F nucleases are used to delete part of a DNA regulatory sequence (e.g., a promoter region) to create an amorphic or hypomorphic allele of a target gene. In certain embodiments the deletion result in a desired hypomorphic allele of the target gene. In certain embodiments, such desired hypomorphic alleles can cause phenotypes which are revealed when the hypomorphic allele is induced (e.g., by a Cas12F nuclease and one or more gRNAs) in a plant cell or plant where the other allele of the target gene is an amorphic allele of the target gene. In certain embodiments, such desired hypomorphic alleles can cause phenotypes which are revealed when the hypomorphic allele is simultaneously induced in both copies of the target gene (e.g., by a Cas12F nuclease and one or more gRNAs) in a plant cell or plant. In some embodiments, the above-described system is optimized such that similar or essentially identical deletions at a target site (e.g., a promoter region) can be made simultaneously in both copies of the target gene, thereby providing a detectable phenotype or change in gene activity. In embodiments, these desired hypomorphic alleles are associated with various phenotypes of interest which are not provided by amorphic alleles

Amorphic alleles of a target gene can be obtained by a variety of techniques and sources. One non-limiting approach to creating amorphic mutations of a target gene is to use CRISPR/RNA-guided endonuclease mutagenesis (e.g., CRISPR/Cas9 mutagenesis) to target exons that encode functional protein domains or to target a large portion (e.g., at least 80%) or the entirety of the coding sequence (see, e.g., Shi et al. Nature Biotechnology. (2015) 33(6): 661-667 and Online Methods). Other mutagenesis techniques can also be used to produce a hypomorphic or amorphic first allele, for example, by introducing mutations in the first allele through transposon insertions, EMS mutagenesis, fast neutron mutagenesis, or other applicable mutagenesis methods. In certain embodiments, amorphic alleles may be present in already characterized and available plant (e.g., soybean or corn) germplasm.

Methods of obtaining a plant cell (e.g., a corn or soybean plant cell) with altered expression of at least one gene comprising: (i) contacting at least one DNA regulatory sequence (e.g., a promoter region) of at least a first allele of the gene in the genome of the plant cell with a Cas12F nuclease and at least one guide RNA that targets the DNA regulatory sequence (e.g., the promoter region), wherein the first allele of the gene is a hypomorphic, isomorphic, or wild-type allele of the gene, and (ii) selecting a plant cell, plant callus, plant part, or plant comprising at least one first deletion in the DNA regulatory sequence (e.g., the promoter region) of the gene that confers the altered expression of the gene are provided. In certain embodiments where the first allele of the targeted gene is an isomorphic or wild-type allele of the gene, the selected plant cell, plant callus, plant part, or plant comprising the deletion in the DNA regulatory sequence (e.g., the promoter region) of the gene exhibits reductions in gene activity resulting from the deletion. In certain embodiments where the first allele of the targeted gene is a hypomorphic allele, the hypomorphic allele can comprise one or more insertions, deletions, or substitutions (INDELS) which provide reductions in gene activity and the selected plant cell, plant callus, plant part, or plant comprising the deletion in the DNA regulatory sequence (e.g., the promoter region) of the gene exhibits further reductions in gene activity resulting from the deletion. In some embodiments, the plant cell further comprises a second allele of the gene which is a hypomorphic, isomorphic, or wild-type allele of the gene, optionally wherein the first allele and the second allele are identical. In other embodiments, the plant cell further comprises the second allele of the gene which is a hypomorphic or amorphic allele of the gene. In some embodiments, the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene. In certain embodiments, the genome contacted with the Cas12F nuclease and the guide RNA comprises a hypomorphic, isomorphic, or wild-type first allele of the gene, the second allele of the gene comprises a hypomorphic or amorphic allele of the gene, and a plant cell, plant callus, plant part, or plant comprising a new hypomorphic allele corresponding to a deletion in the DNA regulatory sequence (e.g., the promoter region) is selected. In other embodiments, the deletion is simultaneously induced in the promoter region of the first and the second allele of the gene and/or a plant cell, plant callus, plant part, or plant comprising the deletion in the promoter region of the first and the second allele of the gene is selected. Also provided are methods wherein the plant cell is a corn plant cell and wherein the corn plant cell is a haploid corn plant cell which contains the first allele of the gene but not the second allele of the gene. In certain embodiments, the genome of the haploid corn plant cell contacted with the Cas12F nuclease and the guide RNA comprises a hypomorphic, isomorphic, or wild-type first and sole allele of the gene, and a corn plant cell, plant callus, plant part, or plant comprising a new hypomorphic allele corresponding to a deletion in the DNA regulatory sequence (e.g., the promoter region) is selected. In certain embodiments of the methods, a plant cell, plant callus, plant part, or plant comprising a deletion in a DNA regulatory sequence (e.g., a promoter region) is selected. In certain embodiments, the selection of a plant cell, plant callus, plant part, or plant containing the deletion comprises: (i) selecting for a phenotype conferred by the altered expression of the gene; (ii) selecting for the altered expression of the gene; (iii) selecting for a plant cell, plant callus, plant part, or plant comprising the gene(s) with the deletion in the promoter region of the first and/or second allele of the gene, or any combination of (i), (ii), or (iii). Phenotypes which can be selected include herbicide resistance; improved tolerance of abiotic stress (e. g., tolerance of temperature extremes, drought, or salt) or biotic stress (e. g., resistance to bacterial or fungal pathogens); improved utilization of nutrients or water; synthesis of new or modified amounts of lipids, carbohydrates, proteins or other chemicals, including medicinal compounds; improved flavor or appearance; improved photosynthesis; improved storage characteristics (e. g., resistance to bruising, browning, or softening); increased yield; altered morphology (e. g., floral architecture or color, plant height, branching, root structure); and changes in flowering time, all in comparison to a control comparison to a control lacking the deletion. Selection for altered expression can comprise selecting for increases in an RNA or protein product produced by the target gene in comparison to a control lacking the deletion. Amounts of a specific RNA can be measured by various methods including quantitative RT-PCR. Amounts of a specific protein can be measured by various methods including immunoassays, mass spectroscopy, and, for proteins with a biochemical activity (e.g., enzymatic activity, binding, etc.). Plant cells comprising a deletion can also be screened for via high-throughput analyses of DNA in the region targeted for deletion. Methods for identifying deletions can be adapted from amplicon and next generation sequencing (NGS) methods that have been used in Targeting Induced Local Lesions in Genomes (TILLING) experiments (Fanelli et al. Sci Rep 11, 9885 (2021). doi.org/10.1038/s41598-021-89237-w).

Also provided are methods wherein distinct guide RNAs targeting distinct promoter regions of the gene are each independently or simultaneously introduced into the plant cell containing the gene to produce a population of plant cells, plant calli, plant parts, or plants comprising distinct deletions in the promoter of the gene and wherein the plant cell, plant callus, plant part, or plant comprising the gene allele with the deletion in the promoter region of the first and/or second allele of the gene and/or altered transcription level is selected from the population of plant cells. In certain embodiments, it can be useful to target distinct promoter regions (e.g. a region of about 10 to 20 or 30 base pairs) of a target gene in parallel or in series and to screen and/or select for one or more deletions of the gene that confer the altered expression of the gene. In certain embodiments, a series of Cas12F gRNAs comprising targeting sequences directing the Cas12F nuclease to different promoter regions are synthesized. In certain embodiments, the series of Cas12F gRNAs can target a promoter regions with Cas12F protospacer adjacent motifs (PAM) that span promoter regions located from any of about 500, 400, 300, 250, or 200 base pairs upstream of the transcriptional start site (TSS) to any of about 100, 50, 20, or 10 base pairs upstream of the transcriptional start site (TSS). In certain embodiments, each gRNA in the series is used to separately used to target a specific promoter region and individual plant cells, calli, or plants are separately screened and/or selected. In other embodiments, two or more distinct gRNAs in the series are introduced into the plant cell simultaneously and individual plant cells, calli, or plants are separately screened and/or selected. In the aforementioned embodiments, the gRNA's can target non-overlapping promoter regions (e.g. a region of about 10 to 20 or 30 base pairs). In certain embodiments, adjacent promoter regions (e.g. a region of about 10 to 20 or 30 base pairs) can be targeted with Cas12F gRNAs directed to those adjacent regions.

In some embodiments of the methods, at least two, three, four, five, six or more different Cas12F gRNAs are used to target a DNA regulatory sequence (e.g., the promoter region) of the plant cell. In some embodiments, any of two or three to any of four, five, six or more different Cas12F gRNAs are used to target a DNA regulatory sequence (e.g., the promoter region) of the plant cell. In certain embodiments, multiple DNA regulatory sequences (e.g., promoter regions) within a single target gene are targeted with at least two Cas12F gRNAs either in parallel (e.g., simultaneously) or in series (e.g., sequentially). In certain embodiments, DNA regulatory sequences (e.g., promoter regions) within at least two target genes are targeted with at least two Cas12F gRNAs either in parallel (e.g., simultaneously) or in series (e.g., sequentially).

In certain embodiments, the Cas12F nuclease used in the methods and plant cells provided herein comprise the polypeptide of any one of SEQ ID NOs: 1, 2, or 3 or a variant thereof having 60% or more sequence identity to SEQ ID NOs: 1, 2, or 3 (e.g., 65% or more, 70% or more, 75% or more, 80% or more, 85% or more, 90% or more, 95% or more, 97% or more, 98% or more, 99% or more, or 100%). In some embodiments, the Cas12F nuclease comprises the polypeptide of any one of SEQ ID NOs: 1, 2, or 3 or a variant thereof having 80% or more sequence identity to SEQ ID NOs: 1, 2, or 3 (e.g., 85% or more, 90% or more, 95% or more, 97% or more, 98% or more, 99% or more, or 100%). In other embodiments, the Cas12F nuclease comprises the polypeptide of any one of SEQ ID NOs: 1, 2, or 3 or a variant thereof having 90% or more sequence identity to SEQ ID NOs: 1, 2, or 3 (e.g., 95% or more, 97% or more, 98% or more, 99% or more, or 100%). In certain embodiments, the Cas12F nuclease comprises the polypeptide of any one of SEQ ID NOs: 1, 2, or 3 or a variant thereof having at least 85%, 90%, 95%, 98%, or 99% sequence identity to SEQ ID NOs: 1, 2, or 3. In certain embodiments, the variant thereof having at least 85%, 90%, 95%, 98%, or 99% sequence identity to SEQ ID NOs: 1, 2, or 3 can comprise, consist essentially of, or consist of conservative amino acid substitutions in SEQ ID NOs: 1, 2, or 3 (e.g., 1, 2, 3, 4, 5, 6, or more conservative amino acid substitutions in SEQ ID NOs: 1, 2, or 3). In some embodiments, the Cas12F nuclease comprises the polypeptide of any one of SEQ ID NOs: 1, 2, or 3.

In some embodiments of the methods and plant cells provided herein, the plant cell is a soybean plant cell and the Cas12F nuclease is encoded by a synthetic DNA molecule having: (i) a GC (guanine and cytosine) content greater than 47% or 48% and/or (ii) a melting temperature (Tm) greater than 89 or 90 degrees Celsius. In certain embodiments, the plant cell is a soybean plant cell and the Cas12F nuclease is encoded by a synthetic DNA molecule having: (i) a GC (guanine and cytosine) content of about 47% or 48% to about 49%, 50%, 51%, 52%, 53%, 54%, 55%, or 56% and/or (ii) a melting temperature (Tm) greater than 89 or 90 degrees Celsius. In some embodiments, the soybean plant cell comprises at least one guide RNA recognized by the Cas12F nuclease, optionally wherein the guide RNA comprises SEQ ID NO: 6, 7, or 8. In certain embodiments, the codon usage of such synthetic DNA molecules comprising the aforementioned Tm and/or GC content(s) is a non-soybean-preferred codon usage (e.g., a majority of the codons used in the synthetic DNA encoding the Cas12F nuclease are codons which are less preferred and/or non-preferred in endogenous soybean genes). Preferred, less-preferred, and non-preferred codons for use in genes in soybean are identified in the soybean codon usage database table for Glycine max (soybean) available at the https website on the world wide web “kazusa.or.jp/codon/.” Methods of synthesizing Cas nuclease encoding synthetic DNAs comprising codons which are less preferred and/or non-preferred in endogenous soybean genes are disclosed in WO2021202397 and U.S. provisional patent applications 63/001,806, 63/072585, and 63/075395, which are all incorporated herein by reference in their entireties. In certain embodiments, the synthetic DNA molecule encoding the Cas12F nuclease comprises SEQ ID NO: 5, SEQ ID NO: 9, SEQ ID NO: 10, SEQ ID NO: 21-28, SEQ ID NO: 37-44, or a variant thereof having at least 60% or more sequence identity to SEQ ID NO: 5, SEQ ID NO: 9, SEQ ID NO: 10, SEQ ID NO: 21-28, SEQ ID NO: 37-44, (e.g., 65% or more, 70% or more, 75% or more, 80% or more, 85% or more, 90% or more, 95% or more, 97% or more, 98% or more, 99% or more, or 100%), optionally wherein the synthetic DNA molecule has: (i) a GC (guanine and cytosine) content greater than 47% or 48% and/or (ii) a melting temperature (Tm) greater than 89 or 90 degrees Celsius. In certain aforementioned embodiments, the guide RNA targets a regulatory sequence (e.g., a promoter region) of a gene in the genome of the soybean plant cell. In certain aforementioned embodiments, the soybean plant cell comprises a first allele of the gene, wherein the first allele of the gene is a hypomorphic, isomorphic, or wild-type allele of the gene. In certain embodiments, the soybean plant cell comprises a second allele of the gene which is a hypomorphic or amorphic allele of the gene. In some embodiments, the soy bean plant cell comprises a first allele of the gene, wherein the first allele of the gene is a hypomorphic, isomorphic, or wild-type allele of the gene and a second allele of the gene is a hypomorphic or amorphic allele of the gene.

Also provided are methods and plant cells wherein the plant cell is a corn plant cell and the Cas12F nuclease is encoded by a synthetic DNA molecule having at least 70%, 75%, 80%, 85%, 90%, 95%, 98%, 99%, or 100% sequence identity to SEQ ID NO: 4, SEQ ID NO: 11, SEQ ID NO: 12, SEQ ID NO: 13-20, or SEQ ID NO: 29-36.

In some embodiments, the promoter region targeted by the Cas12F nuclease is located at the 3′ end of a protospacer adjacent motif (PAM), and the PAM comprises the sequence 5′-TTN, wherein N is A, G, T, or C. In embodiments, said TTN PAM is used to design guide RNAs targeting plant promoters, which are typically abundant in AT base pairs. In some embodiments, alternative PAM site requirements can be engineered into Cas12F variants to modulate PAM-site selectivity and enzyme cutting efficiency (e.g., engineering more relaxed PAM site specificities, engineering more stringent PAM site specificities thereby minimizing off target events, or engineering Cas12F for enhanced enzyme cutting efficiency).

In some embodiments, Cas12F-induced deletions result in altered expression comprising an at least a 2-fold decrease in expression of the gene in comparison to a control lacking the deletion. In some embodiments, altered expression comprises at least a 10% decrease in expression of the gene as compared to a control lacking the deletion, for example a decrease of at least about 20%, or at least about 30%, or at least about 40%, or at least about 50%, or at least about 60%, or at least about 70%, or at least about 80%, or at least about 90% or up to and including a 100% decrease or any decrease between 10-100% as compared to a control lacking the deletion, or at least about a 2-fold, or at least about a 3-fold, or at least about a 4-fold, or at least about a 5-fold or at least about a 10-fold decrease, or any decrease of 2-fold to 10-fold or greater as compared to control lacking the deletion. In certain embodiments, the deletions(s) can confer a decrease in expression of any one of about 2-or 3-fold to any one of about 4-, 5-, 6-, 8-, 10-, 15-, 20-, 40-, or 50-fold in comparison to a control lacking the deletion.

In other embodiments, Cas12F-induced deletions result in altered expression comprising at least a 2-fold increase in expression of the gene in comparison to a control lacking the deletion. In some embodiments, altered expression comprises at least a 10% increase in expression of the gene as compared to a control lacking the deletion, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a control lacking the deletion, or at least about a 2-fold, or at least about a 3-fold, or at least about a 4-fold, or at least about a 5-fold or at least about a 10-fold increase, or any increase of 2-fold to 10-fold or greater as compared to a control lacking the deletion. In certain embodiments, the deletions(s) can confer an increase in expression of any one of about 2-or 3-fold to any one of about 4-, 5-, 6-, 8-, 10-, 15-, 20-, 40-, or 50-fold in comparison to a control lacking the deletion.

Also provided are soybean plant cells, comprising a DNA molecule encoding a Cas12F nuclease, wherein the DNA molecule has: (i) a GC (guanine and cytosine) content greater than 47% or 48% and/or (ii) a melting temperature (Tm) greater than 89 or 90 degrees Celsius. In certain embodiments, the aforementioned synthetic DNA molecule has: (i) a GC content of about 47% or 48% to about 49%, 50%, 51%, 52%, 53%, 54%, 55%, or 56% and/or (ii) a melting temperature (Tm) greater than 89 or 90 degrees Celsius. In certain embodiments, the codon usage of such DNA molecules comprising the aforementioned Tm and/or GC content(s) is a non-soy bean-preferred codon usage (e.g., a majority of the codons used in the synthetic DNA encoding the Cas12F nuclease are codons which are less preferred and/or non-preferred in endogenous soybean genes). Preferred, less-preferred, and non-preferred codons for use in genes in soybean are identified in the soybean codon usage database table for Glycine max (soybean) available at the https website on the world wide web “kazusa.or.jp/codon/.” In some embodiments, the soybean plant cell comprises at least one guide RNA recognized by the Cas12F nuclease, optionally wherein the guide RNA comprises SEQ ID NO: 6, 7, or 8. In other embodiments, the guide RNA targets a regulatory sequence (e.g., a promoter region) of a gene in the genome of the plant cell. In some embodiments, the soybean plant cell comprises a first allele of the gene, wherein the first allele of the gene is a hypomorphic, isomorphic, or wild-type allele of the gene. In certain embodiments, the soybean plant cell comprises a second allele of the gene which is a hypomorphic or amorphic allele of the gene. In some embodiments, the soy bean plant cell comprises a first allele of the gene, wherein the first allele of the gene is a hypomorphic, isomorphic, or wild-type allele of the gene and a second allele of the gene is a hypomorphic or amorphic allele of the gene.

Also provided are soybean plant cells, comprising: (i) a DNA molecule or RNA molecule encoding a Cas12F nuclease; (ii) at least one guide RNA recognized by the Cas12F nuclease, wherein the guide RNA targets a DNA regulatory sequence (e.g., a promoter region) of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene. In some embodiments, the second allele of the gene is an amorphic allele of the gene. In certain embodiments, the amorphic allele comprises: (i) a deletion in or of the promoter region, promoter, and/or coding region of the second allele of the gene; (ii) a nonsense mutation in the coding region of the second allele of the gene; and/or (iii) a frameshift mutation in the second allele of the gene. In certain aforementioned embodiments, the DNA molecule encoding the Cas12F nuclease has: (i) a GC (guanine and cytosine) content greater than 47% or 48% and/or (ii) a melting temperature (Tm) greater than 89 or 90 degrees Celsius. In embodiments, the aforementioned DNA molecule encoding the Cas12F nuclease has: (i) a GC content of about 47% or 48% to about 49%, 50%, 51%, 52%, 53%, 54%, 55%, or 56% and/or (ii) a melting temperature (Tm) greater than 89 or 90 degrees Celsius. In certain embodiments, the codon usage of such DNA molecules comprising the aforementioned Tm and/or GC content(s) is a non-soybean-preferred codon usage (e.g., a majority of the codons used in the synthetic DNA encoding the Cas12F nuclease are codons which are less preferred and/or non-preferred in endogenous soybean genes). Preferred, less-preferred, and non-preferred codons for use in genes in soybean are identified in the soybean codon usage database table for Glycine max (soybean) available at the https website on the world wide web “kazusa.or.jp/codon/.” In certain embodiments, the Cas12F nuclease has RNA-dependent Cas12F nuclease activity. In certain aforementioned embodiments, the DNA molecule comprises a synthetic DNA molecule encoding a Cas12F nuclease having at least 60% sequence identity to SEQ ID NO: 1, SEQ ID NO: 2, or SEQ ID NO: 3 (e.g., 65% or more, 70% or more, 75% or more, 80% or more, 85% or more, 90% or more, 95% or more, 97% or more, 98% or more, 99% or more, or 100%. In certain aforementioned embodiments, the DNA molecule comprises a synthetic DNA molecule encoding a Cas12F nuclease having at least 80% sequence identity to SEQ ID NO: 1, SEQ ID NO: 2, or SEQ ID NO: 3 (e.g., 85% or more, 90% or more, 95% or more, 97% or more, 98% or more, 99% or more, or 100%). In certain aforementioned embodiments, the DNA molecule comprises a synthetic DNA molecule encoding a Cas12F nuclease having at least 90% sequence identity to SEQ ID NO: 1, SEQ ID NO: 2, or SEQ ID NO: 3 (e.g., 95% or more, 97% or more, 98% or more, 99% or more, or 100%). In certain aforementioned embodiments, the Cas12F nuclease comprises the polypeptide of SEQ ID NOs: 1, 2, or 3 or a variant thereof having at least 85%, 90%, 95%, 98%, or 99% sequence identity to SEQ ID NOs: 1, 2, or 3. In certain aforementioned embodiments, the DNA molecule comprises a synthetic DNA molecule having at least 60% sequence identity to SEQ ID NO: 5, 9, 10, 21 to 28, or 37 to 44 (e.g., 65% or more, 70% or more, 75% or more, 80% or more, 85% or more, 90% or more, 95% or more, 97% or more, 98% or more, 99% or more, or 100%). In certain aforementioned embodiments, the DNA molecule of the soybean plant cells comprises a synthetic DNA molecule having at least 80% sequence identity to SEQ ID NO: 5, SEQ ID NO: 9, or SEQ ID NO: 10 (e.g., 85% or more, 90% or more, 95% or more, 97% or more, 98% or more, 99% or more, or 100%). In other embodiments, the DNA molecule of the soybean plant cells comprises a synthetic DNA molecule having at least 90% sequence identity to SEQ ID NO: 5, SEQ ID NO: 9, or SEQ ID NO: 10 (e.g., 95% or more, 97% or more, 98% or more, 99% or more, or 100%). For example, the disclosure provides soybean plant cells wherein the DNA molecule comprises a sequence having at least 85%, 90%, 95%, 98%, or 99% sequence identity to SEQ ID NO: 5, SEQ ID NO: 9, or SEQ ID NO: 10. Soybean callus (e.g., embryogenic callus), soybean plant parts, and soybean plants comprising any of the aforementioned soybean plant cells are also provided. In certain embodiments, the soybean plant part is a leaf, stem, root, pod, or seed.

Corn plant cells comprising a synthetic DNA molecule having at least 60% sequence identity to SEQ ID NO: 4, SEQ ID NO: 11, SEQ ID NO: 12, SEQ ID NO: 13-20, or SEQ ID NO: 29-36 (e.g., 65% or more, 70% or more, 75% or more, 80% or more, 85% or more, 90% or more, 95% or more, 97% or more, 98% or more, 99% or more, or 100%) are provided. In other embodiments, corn plant cells comprise a synthetic DNA molecule having at least 80% sequence identity to SEQ ID NO: 4, SEQ ID NO: 11, SEQ ID NO: 12, SEQ ID NO: 13-20, or SEQ ID NO: 29-36 (e.g., 85% or more, 90% or more, 95% or more, 97% or more, 98% or more, 99% or more, or 100%) are provided. In other embodiments, corn plant cells comprise a synthetic DNA molecule having at least 90% sequence identity to SEQ ID NO: 4, SEQ ID NO: 11, SEQ ID NO: 12, SEQ ID NO: 13-20, or SEQ ID NO: 29-36 (e.g., 95% or more, 97% or more, 98% or more, 99% or more, or 100%). Also provided are corn plant cells comprising a synthetic DNA molecule having at least 85%, 90%, 95%, 98%, 99%, or 100% sequence identity to SEQ ID NO: 4, SEQ ID NO: 11, SEQ ID NO: 12, SEQ ID NO: 13-20, or SEQ ID NO: 29-36. In other embodiments, the corn plant cell further comprises at least one guide RNA recognized by a Cas12F nuclease, optionally wherein the guide RNA comprises SEQ ID NO: 6, 7, or 8.

Corn plant cells comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the corn plant cell, wherein the first allele of the gene is a hypomorphic, isomorphic, or wild-type allele of the gene; and (iii) a second allele of the gene which is a hypomorphic or amorphic allele of the gene are provided. In some embodiments, the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene. In certain embodiments, the corn plant cell is a haploid corn plant cell which contains the first allele of the gene but not the second allele of the gene. In certain aforementioned embodiments, the DNA molecule of the corn plant cell comprises a synthetic DNA molecule having at least 60% or more sequence identity to SEQ ID NO: 4, SEQ ID NO: 11, SEQ ID NO: 12, SEQ ID NO: 13-20, or SEQ ID NO: 29-36 (e.g., 65% or more, 70% or more, 75% or more, 80% or more, 85% or more, 90% or more, 95% or more, 97% or more, 98% or more, 99% or more, or 100%). In certain embodiments, the Cas12F nuclease has RNA-dependent Cas12F nuclease activity. In certain aforementioned embodiments, the Cas12F nuclease comprises the polypeptide of any one of SEQ ID NOs: 1, 2, or 3 or a variant thereof having 60% or more sequence identity to SEQ ID NOs: 1, 2, or 3 (e.g., 65% or more, 70% or more, 75% or more, 80% or more, 85% or more, 90% or more, 95% or more, 97% or more, 98% or more, 99% or more, or 100%). Corn callus (e.g. embryogenic callus), corn plant parts, and corn plants comprising any of the aforementioned corn plant cells are also provided. In certain embodiments, the corn plant part is a leaf, stem, root, or seed.

Various methods can be used to introduce one or more polynucleotides encoding a Cas12F nuclease protein and/or polynucleotides encoding or comprising a Cas12F nuclease guide RNA into a host cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. In plants, bacterially mediated (e.g., Agrobacterium sp., Rhizobium sp., Sinorhizobium sp., Mesorhizobium sp., Bradyrhizobium sp., Azobacter sp., Phyllobacterium sp.) transfection or transformation of a plant (e.g., soybean or corn) cell, protoplast, embryo, callus, or tissue with a nucleic acid comprising the polynucleotides can be used; see, e.g., Broothaerts et al. (2005) Nature, 433:629-633. Introducing a recombinant expression vector (e.g., a plasmid for soybean expression or corn expression) into cells can occur in any culture media and under any culture conditions that promote the survival of the cells. Introducing said recombinant expression vector into a target cell can be carried out in vivo or ex vivo. Introducing the recombinant expression vector into a target cell can be carried out in vitro.

In some embodiments, a polynucleotide encoding a Cas12F nuclease protein can be provided as RNA. The RNA can be provided by direct chemical synthesis or can be transcribed in vitro from a DNA (e.g., encoding the Cas12F nuclease protein). Once synthesized, the RNA can be introduced into a cell by any of the well-known techniques for introducing nucleic acids into cells (e.g., microinjection, electroporation, transfection, etc.). In certain embodiments, Cas12F gRNAs can also be introduced as RNA molecules or as ribonucleoprotein particles (e.g., as Cas12F nucleases complexed with the Cas12F gRNA).

Nucleic acids can be provided to the cells using well-developed transfection techniques; see, e.g., Angel and Yanik (2010) PLOS ONE 5(7): el 1756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50): 19821-19826.

Vectors can be provided directly to a target host cell (e.g., a soybean plant cell or corn plant cell). In other words, the cells are contacted with vectors comprising the polynucleotides (e.g., recombinant expression vectors encoding the Cas12F nuclease protein; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors (e.g., gemini virus vectors, TMV vectors, and the like) containing the polynucleotides.

Vectors used for providing the nucleic acids encoding Cas12F nuclease guide RNA and/or a Cas12F nuclease polypeptide to a target host cell can include suitable promoters for driving the expression, that is, transcriptional activation, of the gene of interest. In other words, in some cases, the nucleic acid of interest will be operably linked to a promoter. This can include ubiquitously acting promoters, for example, a viral promoter (e.g., CaMV35S or CaMV19S), actin promoter, or inducible promoters, such as promoters that are active in particular cell populations or that respond to the presence of drugs such as tetracycline. In addition, vectors used for providing a nucleic acid encoding a Cas12F nuclease guide RNA and/or a Cas12F nuclease protein to a cell can include nucleic acid sequences that encode selectable markers in the target cells, so as to identify cells that have taken up the vector encoding the Cas12F nuclease guide RNA and/or Cas12F nuclease protein.

Any of a variety of compounds, vector systems (e.g., bacterial plant transformation vector systems), and methods can be used to deliver to a target cell (e.g., a plant cell including a soybean plant cell or a corn plant cell) a Cas12F nuclease system comprising a polynucleotide. A Cas12F nuclease system provided herein includes systems which can comprise: (a) a polynucleotide encoding a Cas12F nuclease polypeptide (e.g., Cas12f.4, Cas12f.5, Cas12f.6, or variant thereof), and a Cas12F nuclease guide RNA; (b) a polynucleotide comprising an mRNA encoding a Cas12F nuclease polypeptide, and a Cas12F nuclease guide RNA; (c) a recombinant expression vector comprising a polynucleotide encoding a Cas12F nuclease polypeptide and a polynucleotide sequence encoding a Cas12F nuclease guide RNA; (d) a first recombinant expression vector comprising a polynucleotide encoding a Cas12F nuclease polypeptide, and a second recombinant expression vector comprising a nucleotide sequence encoding a Cas12F nuclease guide RNA; (e) a Cas12F nuclease polypeptide, and a Cas12F nuclease guide RNA; or (f) a recombinant expression vector comprising a polynucleotide encoding a Cas12F nuclease polypeptide, a nucleotide sequence encoding a first Cas12F nuclease guide RNA, and a nucleotide sequence encoding a second Cas12F nuclease guide RNA; or some variation of one of (a) through (f). As a non-limiting example, a Cas12F nuclease system can be combined with a lipid. As another non-limiting example, a Cas12F nuclease system can be combined with a particle, or formulated into a particle. As another non-limiting example, a Cas12F nuclease system can be contained in or delivered to a plant cell (e.g., a soybean plant cell and/or a corn plant cell).

In some cases, a nucleic acid (e.g., a Cas12F nuclease guide RNA; a nucleic acid comprising a polynucleotide encoding a Cas12F nuclease polypeptide; one or more components of a Cas12F nuclease system, etc.) is delivered to a cell (e.g., a target host cell such as a soybean plant cell or corn plant cell) in a particle or associated with a particle. In some cases, a Cas12F nuclease system is delivered to a cell in a particle or associated with a particle. The terms “particle” and nanoparticle” can be used interchangeable, as appropriate. A recombinant expression vector comprising a polynucleotide encoding a Cas12F nuclease polypeptide of the present disclosure, and guide RNA can be delivered simultaneously using particles or lipid envelopes. For instance, a polynucleotide encoding a Cas12F nuclease polypeptide and a Cas12F nuclease guide RNA can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

An mRNA comprising a polynucleotide encoding a Cas12F nuclease polypeptide or a recombinant expression vector comprising a polynucleotide encoding a Cas12F nuclease polypeptide and/or Cas12F nuclease guide RNA (or a nucleic acid such as one or more expression vectors encoding the Cas12F nuclease guide RNA) can be delivered simultaneously using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used.

Supercharged proteins can be used to deliver a polynucleotide or one or more components of a Cas12F nuclease system to a target cell. Supercharged proteins are a class of engineered or naturally-occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a polynucleotide or one or more components of a Cas12F nuclease system to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

As described above, in some cases, a nucleic acid (e.g., a recombinant expression vector) comprising a polynucleotide encoding a Cas12F nuclease is used as a transgene to generate a transgenic plant that produces a Cas12F nuclease polypeptide. Transgenic plants, plant parts (e.g., seed), tissues, or transgenic plant cell, and particularly a transgenic corn plant, corn plant part, soybean plant, soybean plant part (e.g., soybean seed), soybean tissue, or transgenic soybean plant cell comprising a polynucleotide encoding a Cas12F nuclease polypeptide are provided. In some embodiments, the genome of the transgenic plant comprises a polynucleotide. In some embodiments, the transgenic plant is homozygous for the genetic modification. In some embodiments, the transgenic plant is heterozygous for the genetic modification. Methods set forth in Schindele et al. (FEBS Letters 592(2018) 1954-1967) for use of Cas9 or Cas12-based nuclease in plants can be adapted for use with the polynucleotides provided herein.

### EMBODIMENTS

Various embodiments of the plant cells and methods provided herein are included in the following non-limiting list of embodiments.
1. A method of obtaining a plant cell with altered expression of at least one gene comprising:
   (i) contacting at least one DNA regulatory sequence of at least a first allele of the gene in the genome of the plant cell with a Cas12F nuclease and at least one guide RNA that targets the DNA regulatory sequence, wherein the first allele of the gene is a hypomorphic, isomorphic, or wild-type allele of the gene, optionally wherein the DNA regulatory sequence is a promoter region, and
   (ii) selecting a plant cell, plant callus, plant part, or plant comprising at least one first deletion in the DNA regulatory sequence of the gene that confers the altered expression of the gene.
2. The method of embodiment 1, wherein the plant cell comprises a second allele of the gene which is a hypomorphic, isomorphic, or wild-type allele of the gene, optionally wherein the first allele and the second allele are identical.
3. The method of embodiment 1, wherein the plant cell further comprises the second allele of the gene which is a hypomorphic or amorphic allele of the gene.
4. The method of embodiment 3, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.
5. The method of embodiment 1, wherein the first deletion occurs in the promoter region of the first and the second allele of the gene.
6. The method of embodiment 1, wherein the DNA regulatory sequence is a promoter region and wherein distinct guide RNAs targeting distinct promoter regions of the gene are each independently introduced into the plant cell containing the gene to produce a population of plant cells, plant calli, plant parts, or plants comprising distinct deletions in the promoter of the gene and wherein the plant cell, plant callus, plant part, or plant comprising the gene allele with the deletion in the promoter region of the first and/or second allele of the gene and/or altered transcription level is selected from the population of plant cells.
7. The method of any one of embodiments 1 to 6, wherein the Cas12F nuclease comprises Cas12f.4 nuclease, Cas12f.5 nuclease, Cas12f.6 nuclease, or an engineered Cas12F nuclease.
8. The method of any one of embodiments 1 to 7, wherein the Cas12F nuclease comprises the polypeptide of any one of SEQ ID NOs: 1, 2, or 3 or a variant thereof having at least 85%, 90%, 95%, 98%, or 99% sequence identity to SEQ ID NOs: 1, 2, or 3.
9. The method of any one of embodiments 1 to 8, wherein the plant cell is a soybean plant cell and the Cas12F nuclease is encoded by a synthetic DNA molecule having: (i) a GC (guanine and cytosine) content greater than 47% or 48% and/or (ii) a melting temperature (Tm) greater than 89 or 90 degrees Celsius.
10. The method of embodiment 9, wherein the synthetic DNA molecule comprises SEQ ID NO: 5, SEQ ID NO: 9, SEQ ID NO: 10, or a variant thereof having at least 80%, 85%, 90%, 95%, 98%, or 99% sequence identity to SEQ ID NO:5, SEQ ID NO: 9, SEQ ID NO: 10, optionally wherein the synthetic DNA molecule has: (i) a GC (guanine and cytosine) content greater than 47% or 48% and/or (ii) a melting temperature (Tm) greater than 89 or 90 degrees Celsius.
11. The method of any one of embodiments 1 to 8, wherein the plant cell is a corn plant cell and wherein the corn plant cell is a haploid corn plant cell which contains the first allele of the gene but not the second allele of the gene.
12. The method of any one of embodiments 1 to 8, wherein the plant cell is a corn plant cell and the Cas12F nuclease is encoded by a synthetic DNA molecule having at least 85%, 90%, 95%, 98%, 99%, or 100% sequence identity to SEQ ID NO: 4, 11 to 20, or 29 to 36.
13. The method of any one of embodiments 1 to 12, wherein the promoter region is located at the 3′ end of a protospacer adjacent motif (PAM), and the PAM comprises the sequence 5′-TTN, wherein N is A, G, T, or C.
14. The method of any one of embodiments 1 to 13, wherein the at least one guide RNA comprises SEQ ID NO: 6, 7, 8, or a variant thereof recognized by the Cas12F nuclease.
15. The method of any one of embodiments 1 to 14, wherein the Cas12F nuclease is encoded by a DNA molecule having at least 95%, 98%, or 99% sequence identity to SEQ ID NO: 4, 5, or 9 to 44.
16. The method of any one of embodiments 1 to 15, wherein the deletion(s) comprise about 10 to 20 base pairs of DNA in the promoter region.
17. The method of any one of embodiments 1 to 16, wherein the altered expression comprises at least a 2-fold decrease in expression of the gene in comparison to a control lacking the deletion.
18. The method of any one of embodiments 1 to 16, wherein the altered expression comprises at least a 2-fold increase in expression of the gene in comparison to a control lacking the deletion.
19. The method of any one of embodiments 1 to 18, wherein the selecting comprises: (i) selecting for a phenotype conferred by the altered expression of the gene; (ii) selecting for the altered expression of the gene; (iii) selecting for a plant cell, plant callus, plant part, or plant comprising the gene(s) with the deletion in the promoter region of the first and/or second allele of the gene, or any combination of (i), (ii), or (iii).
20. A soybean plant cell comprising a DNA molecule encoding a Cas12F nuclease, wherein the DNA molecule has: (i) a GC (guanine and cytosine) content greater than 47% or 48% and/or (ii) a melting temperature (Tm) greater than 89 or 90 degrees Celsius.
21. The soybean plant cell of embodiment 20, wherein the soybean plant cell further comprises at least one guide RNA recognized by the Cas12F nuclease, optionally wherein the guide RNA comprises SEQ ID NO: 6, 7, 8, or a variant thereof recognized by the Cas12F nuclease.
22. The soybean plant cell of embodiment 21, wherein the guide RNA targets a promoter region of a gene in the genome of the plant cell.
23. The soybean plant cell of embodiment 22, wherein the soybean plant cell comprises a first allele of the gene, wherein the first allele of the gene is a hypomorphic, isomorphic, or wild-type allele of the gene.
24. The soybean plant cell of embodiment 23, wherein the soybean plant cell comprises a second allele of the gene which is a hypomorphic or amorphic allele of the gene.
25. A soybean plant cell comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.
26. The soybean plant cell of embodiment 25, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.
27. The soybean plant cell of embodiment 25 or 26, wherein the DNA molecule encoding the Cas12F nuclease has: (i) a GC (guanine and cytosine) content greater than 47% or 48% and/or (ii) a melting temperature (Tm) greater than 89 or 90 degrees Celsius.
28. The soybean plant cell of any one of embodiments 20 to 27, wherein the Cas12F nuclease has RNA-dependent Cas12F nuclease activity.
29. The soybean plant cell of any one of embodiments 20 to 28, wherein the Cas12F nuclease comprises the polypeptide of SEQ ID NOs: 1, 2, or 3 or a variant thereof having at least 85%, 90%, 95%, 98%, or 99% sequence identity to SEQ ID NOs: 1, 2, or 3.
30. The soybean plant cell of any one of embodiments 20 to 29, wherein the DNA molecule comprises a sequence having at least 85%, 90%, 95%, 98%, or 99% sequence identity to SEQ ID NO: 5, 9, 10, 21 to 28, or 37 to 44.
31. Soybean callus comprising the soybean plant cell of any one of embodiments 20 to 30.
32. A soybean plant part comprising the soybean plant cell of any one of embodiments 20 to 30, optionally wherein the plant part is selected from a leaf, stem, root, pod, or seed.
33. A soybean plant comprising the soybean plant cell of any one of embodiments 20 to 30.
34. A corn plant cell comprising a synthetic DNA molecule having at least 85%, 90%, 95%, 98%, 99%, or 100% sequence identity to SEQ ID NO: 4, 11 to 20, or 29 to 36.
35. The corn plant cell of embodiment 34, wherein the corn plant cell further comprises at least one guide RNA recognized by a Cas12F nuclease, optionally wherein the guide RNA comprises SEQ ID NO: 6, 7, 8, or a variant thereof recognized by the Cas12F nuclease.
36. A corn plant cell comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.
37. The corn plant cell of embodiment 36, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.
38. The corn plant cell of embodiment 37, wherein the corn plant cell is a haploid corn plant cell which contains the first allele of the gene but not the second allele of the gene.
39. The corn plant cell of embodiment 37, wherein the DNA molecule comprises a synthetic DNA molecule having at least 85%, 90%, 95%, 98%, 99%, or 100% sequence identity to SEQ ID NO: 4, 11 to 20, or 29 to 36.
40. The corn plant cell of any one of embodiments 35 to 39, wherein the Cas12F nuclease has RNA-dependent Cas12F nuclease activity.
41. The corn plant cell of any one of embodiments 36 to 37, wherein the Cas12F nuclease comprises the polypeptide of SEQ ID NOs: 1, 2, or 3 or a variant thereof having at least 85%, 90%, 95%, 98%, or 99% sequence identity to SEQ ID NO: 1, 2, or 3.
42. Corn callus comprising the corn plant cell of any one of embodiments 34 to 41.
43. A corn plant part comprising the corn plant cell of any one of embodiments 34 to 41, optionally wherein the plant part is selected from a leaf, stem, root, pod, or seed.
44. A corn plant comprising the corn plant cell of any one of embodiments 34 to 41.

### EXAMPLES

The following examples are not intended to limit the scope of the claims.

### Example 1—Cas12F Sequence Construction and Sequencing Results of Deletions in Corn and Soybean Cells

### Sequence Construction

A plasmid (pIN2842) for soybean expression was made containing the following expression cassettes: SlUB10p::NLS-SoyCas12f.4-NLS::HSPt, and HaUB10p::mgfp6::pearbscE9t.

Soybean protoplasts were made and PEG-transfected with the pIN2842 plasmid, plus one mature guide RNA. The guide RNA was selected from twelve different possible guide RNAs. Each guide RNA for the twelve soybean genome targets was synthesized in its mature form. A Cas12 of a different subtype was also tested for comparison. Guide DNA (“gDNA”) was extracted from the protoplast cells and an amplicon of the target region was sequenced.

A plasmid (pIN2915) for corn expression was made containing the following expression cassettes: ZmUBI1p::CornCas12f.4-2xNLS::HSPt, and OsUBIp::mGFP6::Pea_rbsc_E9t. Guide RNAs for twelve corn genome targets were synthesized in their mature form. Corn protoplasts were made, and PEG-transfected with the pIN2915 plasmid and one mature guide RNA. A Cas12 of a different subtype was also tested for comparison.

Soybean protoplasts were made and PEG-transfected with the Cas-expressing plasmid and purified guide RNA. Guide DNA (“gDNA”) was extracted from the protoplast cells and an amplicon of the target region was sequenced.

### Sequencing Results Showing Deletion Size and Relative Frequency

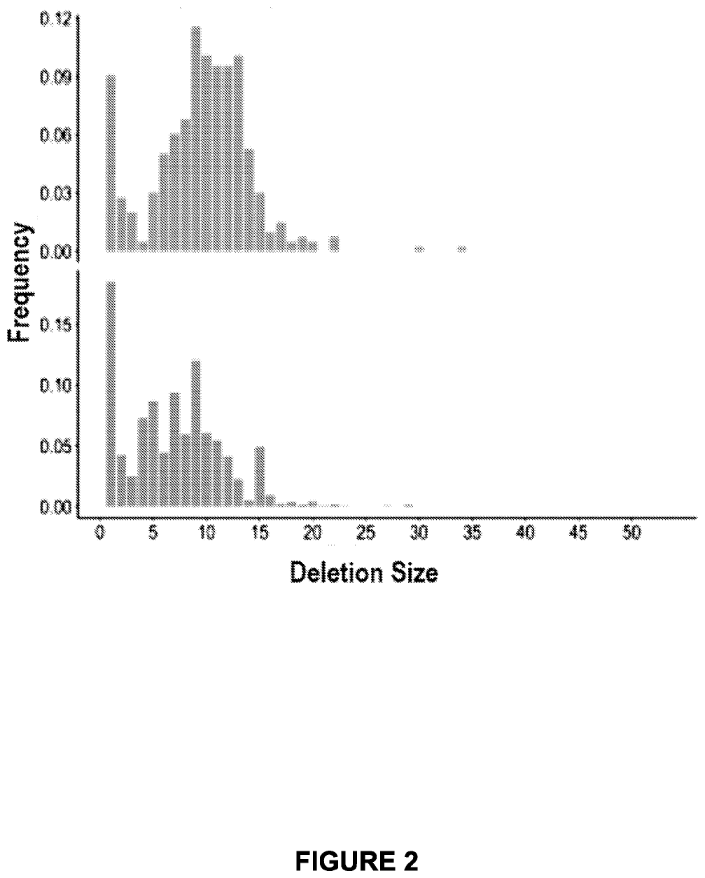

Combined corn and soybean sequencing results of deletions are illustrated in FIG. 2. FIG. 2 shows the distribution of deletion sizes at edited target sites for Cas12f.4 (top) and control (bottom). The data represent 90 samples from soybean and corn Cas12f.4 editing tests, and 500 control samples.

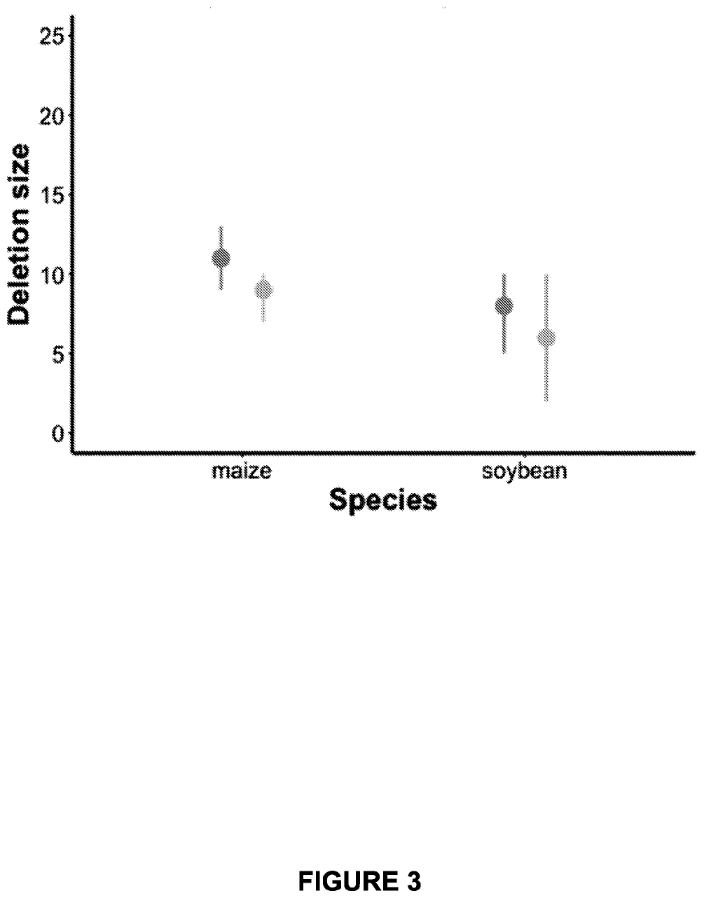

Cas12f.4 edits show a size distribution of 1, 7, 10, 12, and 34 nucleotides at the respectively minimum, 25th percentile, median, 75th percentile, and maximum points of the range. The corresponding control distribution of deletion size values was observed at 1, 3, 7, 10, and 53 nucleotides. Deletion edits made up about 95% of all edits for both Cas12f.4 and controls. FIG. 3 illustrates the distribution of the interquartile ranges of the measurements for corn and soybean deletions. Specifically, FIG. 3 shows the median and IQR of deletion sizes in maize and soybean caused by Cas12f.4 (left) and control (right) nucleases. Table 2 below describes the same data. As shown in Table 2, Cas12f.4 produces longer deletions more frequently than the control

TABLE 2

| | | Quantile | | | Quantile | |
|---|---|---|---|---|---|---|
| Species | Min | 25 | Median | Mean | 75 | Max |
| corn test | 1 | 9 | 11 | 11.35 | 13 | 34 |
| corn | 1 | 7 | 9 | 8.67 | 10 | 53 |
| control | | | | | | |
| soybean | 1 | 5 | 8 | 7.75 | 10 | 30 |
| test | | | | | | |
| soybean | 1 | 2 | 6 | 6.58 | 10 | 45 |
| control | | | | | | |

Although the foregoing disclosure has been described in some detail by way of illustration and example for purposes of clarity of understanding, it will be clear that certain changes and modifications can be practiced within the scope of the appended claims.

What is claimed is:

1. A method of obtaining a plant cell with altered expression of at least one gene comprising:
(i) contacting at least one promoter region of at least a first allele of the gene in the genome of the plant cell with a Cas12F nuclease and at least one guide RNA that targets the promoter region, wherein the first allele of the gene is a hypomorphic, isomorphic, or wild-type allele of the gene, and
(ii) selecting a plant cell, plant callus, plant part, or plant comprising at least one first deletion in the promoter of the gene that confers the altered expression of the gene.

2. The method of claim 1, wherein the plant cell comprises a second allele of the gene which is a hypomorphic, isomorphic, or wild-type allele of the gene, optionally wherein the first allele and the second allele are identical.

3. The method of claim 1, wherein the plant cell further comprises the second allele of the gene which is a hypomorphic or amorphic allele of the gene.

4. The method of claim 3, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.

5. The method of claim 1, wherein the first deletion occurs in the promoter region of the first and the second allele of the gene.

6. The method of claim 1, wherein distinct guide RNAs targeting distinct promoter regions of the gene are each independently introduced into the plant cell containing the gene to produce a population of plant cells, plant calli, plant parts, or plants comprising distinct deletions in the promoter of the gene and wherein the plant cell, plant callus, plant part, or plant comprising the gene allele with the deletion in the promoter region of the first and/or second allele of the gene and/or altered transcription level is selected from the population of plant cells.

7. The method of claim 6, wherein the Cas12F nuclease comprises Cas12f.4 nuclease, Cas12f.5 nuclease, Cas12f.6 nuclease, or an engineered Cas12F nuclease.

8. The method of claim 7, wherein the Cas12F nuclease comprises the polypeptide of any one of SEQ ID NOs: 1, 2, or 3 or a variant thereof having at least 85%, 90%, 95%, 98%, or 99% sequence identity to SEQ ID NOs: 1, 2, or 3.

9. The method of claim 7, wherein the plant cell is a soybean plant cell and the Cas12F nuclease is encoded by a synthetic DNA molecule having: (i) a GC (guanine and cytosine) content greater than 47% or 48% and/or (ii) a melting temperature (Tm) greater than 89 or 90 degrees Celsius.

10. The method of claim 9, wherein the synthetic DNA molecule comprises SEQ ID NO: 5, SEQ ID NO: 9, SEQ ID NO: 10, or a variant thereof having at least 80%, 85%, 90%, 95%, 98%, or 99% sequence identity to SEQ ID NO:5, SEQ ID NO: 9, SEQ ID NO: 10, optionally wherein the synthetic DNA molecule has: (i) a GC (guanine and cytosine) content greater than 47% or 48% and/or (ii) a melting temperature (Tm) greater than 89 or 90 degrees Celsius.

11. The method of claim 7, wherein the plant cell is a corn plant cell and wherein the corn plant cell is a haploid corn plant cell which contains the first allele of the gene but not the second allele of the gene.

12. The method of claim 7, wherein the plant cell is a corn plant cell and the Cas12F nuclease is encoded by a synthetic DNA molecule having at least 85%, 90%, 95%, 98%, 99%, or 100% sequence identity to SEQ ID NO: 4, SEQ ID NO: 11 to 20, or SEQ ID NO: 29 to 36.

13. The method of any one of claims 1 to 12, wherein the promoter region is located at the 3′ end of a protospacer adjacent motif (PAM), and the PAM comprises the sequence 5′-TTN, wherein N is A, G, T, or C.

14. The method of any one of claims 1 to 12, wherein the at least one guide RNA comprises SEQ ID NO: 6, 7, or 8.

15. The method of any one of claims 1 to 6, wherein the Cas12F nuclease is encoded by a DNA molecule having at least 95%, 98%, or 99% sequence identity to SEQ ID NO: 4, SEQ ID NO: 5, or SEQ ID NO: 9 to 44.

16. The method of any one of claims 1 to 12, wherein the deletion(s) comprise about 10 to 20 base pairs of DNA in the promoter region.

17. The method of any one of claims 1 to 12, wherein the altered expression comprises at least a 2-fold decrease in expression of the gene in comparison to a control lacking the deletion.

18. The method of any one of claims 1 to 12, wherein the altered expression comprises at least a 2-fold increase in expression of the gene in comparison to a control lacking the deletion.

19. The method of any one of claims 1 to 12, wherein the selecting comprises: (i) selecting for a phenotype conferred by the altered expression of the gene; (ii) selecting for the altered expression of the gene; (iii) selecting for a plant cell, plant callus, plant part, or plant comprising the gene(s) with the deletion in the promoter region of the first and/or second allele of the gene, or any combination of (i), (ii), or (iii).

20. A soybean plant cell comprising a DNA molecule encoding a Cas12F nuclease, wherein the DNA molecule has: (i) a GC (guanine and cytosine) content greater than 47% or 48% and/or (ii) a melting temperature (Tm) greater than 89 or 90 degrees Celsius.

21. The soybean plant cell of claim 20, wherein the soybean plant cell further comprises at least one guide RNA recognized by the Cas12F nuclease, optionally wherein the guide RNA comprises SEQ ID NO: 6, 7, or 8.

22. The soybean plant cell of claim 21, wherein the guide RNA targets a promoter region of a gene in the genome of the plant cell.

23. The soybean plant cell of claim 22, wherein the soybean plant cell comprises a first allele of the gene, wherein the first allele of the gene is a hypomorphic, isomorphic, or wild-type allele of the gene.

24. The soybean plant cell of claim 23, wherein the soybean plant cell comprises a second allele of the gene which is a hypomorphic or amorphic allele of the gene.

25. A soybean plant cell comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.

26. The soybean plant cell of claim 25, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.

27. The soybean plant cell of claim 25, wherein the DNA molecule encoding the Cas12F nuclease has: (i) a GC (guanine and cytosine) content greater than 47% or 48% and/or (ii) a melting temperature (Tm) greater than 89 or 90 degrees Celsius.

28. The soybean plant cell of any one of claims 20 to 27, wherein the Cas12F nuclease has RNA-dependent Cas12F nuclease activity.

29. The soybean plant cell of any one of claims 25 to 27, wherein the Cas12F nuclease comprises the polypeptide of SEQ ID NOs: 1, 2, or 3 or a variant thereof having at least 85%, 90%, 95%, 98%, or 99% sequence identity to SEQ ID NOs: 1, 2, or 3.

30. The soybean plant cell of any one of claims 25 to 27, wherein the DNA molecule comprises a sequence having at least 85%, 90%, 95%, 98%, or 99% sequence identity to SEQ ID NO: 5, SEQ ID NO: 9, SEQ ID NO: 10, SEQ ID NO: 21 to 28, or SEQ ID NO: 37 to 44.

31. Soybean callus comprising the soybean plant cell of any one of claims 20 to 27.

32. A soybean plant part comprising the soybean plant cell of any one of claims 20 to 27, optionally wherein the plant part is selected from a leaf, stem, root, pod, or seed.

33. A soybean plant comprising the soybean plant cell of any one of claims 20 to 27.

34. A corn plant cell comprising a synthetic DNA molecule having at least 85%, 90%, 95%, 98%, 99%, or 100% sequence identity to SEQ ID NO: 4, SEQ ID NO: 11 to 20, or SEQ ID NO: 29 to 36.

35. The corn plant cell of claim 34, wherein the corn plant cell further comprises at least one guide RNA recognized by a Cas12F nuclease, optionally wherein the guide RNA comprises SEQ ID NO: 6, 7, or 8.

36. A corn plant cell comprising: (i) a DNA molecule or RNA molecule encoding a Cas12F nuclease; (ii) at least one guide RNA recognized by the Cas12F nuclease, wherein the guide RNA targets a promoter region of at least a first allele of a gene in the genome of the soybean plant cell, wherein the first allele of the gene is a hypomorphic, isomorphic, or wild-type allele of the gene; and (iii) a second allele of the gene which is a hypomorphic or amorphic allele of the gene.

37. The corn plant cell of claim 36, wherein the second allele of the gene is an amorphic allele of the gene, optionally wherein the amorphic allele comprises: (i) a second deletion in or of the promoter region, promoter, and/or coding region of the second allele of the gene; (ii) a nonsense mutation in the coding region of the second allele of the gene; and/or (iii) a frameshift mutation in the second allele of the gene.

38. The corn plant cell of claim 37, wherein the corn plant cell is a haploid corn plant cell which contains the first allele of the gene but not the second allele of the gene.

39. The corn plant cell of claim 37, wherein the DNA molecule comprises a synthetic DNA molecule having at least 85%, 90%, 95%, 98%, 99%, or 100% sequence identity to SEQ ID NO: 4, SEQ ID NO: 11 to 20, or SEQ ID NO: 29 to 36.

40. The corn plant cell of any one of claims 35 to 39, wherein the Cas12F nuclease has RNA-dependent Cas12F nuclease activity.

41. The corn plant cell of any one of claims 36 to 37, wherein the Cas12F nuclease comprises the polypeptide of SEQ ID NOs: 1, 2, or 3 or a variant thereof having at least 85%, 90%, 95%, 98%, or 99% sequence identity to SEQ ID NO: 1, 2, or 3.

42. Corn callus comprising the corn plant cell of any one of claims 34 to 39.

43. A corn plant part comprising the corn plant cell of any one of claims 34 to 39, optionally wherein the plant part is selected from a leaf, stem, root, pod, or seed.

44. A corn plant comprising the corn plant cell of any one of claims 34 to 39.
